(19) United States
(12) Patent Application Publication
(10) Pub. No.: US 2011/0319424 A1
(43) Pub. Date: Dec. 29, 2011

(54) NAPHTHYRIDIN-2(1H) -ONE COMPOUNDS USEFUL AS ANTIBACTERIALS

(76) Inventor: Carlos ALEMPARTE-GALLARDO, Middlesex (GB)

(21) Appl. No.: 12/863,254

(22) PCT Filed: Jan. 15, 2009
(86) PCT No.: PCT/EP09/50436

§ 371 Date: Jul. 16, 2010

(30) Foreign Application Priority Data
Jan. 18, 2008 (EP) 08382002.7

(51) Int. Cl. A61K 31/4375; A61P 31/06; A61K 31/497; A61K 31/506; C07D 471/04; A61K 31/501

(52) U.S. Cl. 514/255.05; 546/122; 544/238; 544/405; 544/333; 514/300; 514/247; 514/256

(57) ABSTRACT

Compounds of Formula (I),

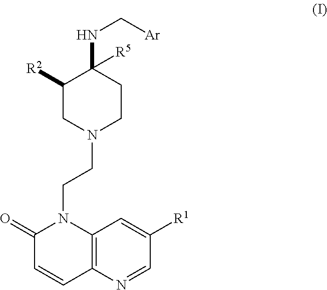

wherein substituents R¹, R² and R⁵ are as defined, and Ar represents substituted phenyl, pyridyl, pyridazinyl, pyrimidinyl, pyrazinyl, thiazolyl, furanyl, imidazolyl and thiophenyl; compositions containing them, their use in therapy, including their use as antibacterials, for example in the treatment of tuberculosis, and methods for the preparation of such compounds, are provided.

### FIELD OF THE INVENTION

This invention relates to compounds, compositions containing them, their use in therapy, including their use as antibacterials, for example in the treatment of tuberculosis, and methods for the preparation of such compounds.

### BACKGROUND OF THE INVENTION

PCT patent publications WO02/08224, WO02/50061, WO02/56882, WO02/96907, WO2003087098, WO2003010138, WO2003064421, WO2003064431, WO2004002992, WO2004002490, WO2004014361, WO2004041210, WO2004096982, WO2002050036, WO2004058144, WO2004087145, WO2006002047, WO2006014580, WO2006010040, WO2006017326, WO2006012396, WO2006017468, WO2006020561, WO2006081179, WO2006081264, WO2006081289, WO2006081178, WO2006081182, WO01/25227, WO02/40474, WO02/07572, WO2004024712, WO2004024713, WO2004035569, WO2004087647, WO2004089947, WO2005016916, WO2005097781, WO2006010831, WO2006021448, WO2006032466, WO2006038172, WO2006046552, WO2006099884, WO2006105289, WO2006081178, WO2006081182, WO2007016610, WO2007081597, WO2007071936, WO2007115947, WO2007118130 and WO2007122258 disclose quinoline, naphthyridine, morpholine, cyclohexane, piperidine and piperazine derivatives and also tricyclic condensed ring compounds, having antibacterial activity. WO2004104000 discloses tricyclic condensed ring compounds capable of selectively acting on cannabinoid receptors.

Synthetic drugs for treating tuberculosis (TB) have been available for over half a century, but incidences of the disease continue to rise world-wide. In 2004, it is estimated that 24,500 people developed active disease and close to 5,500 died each day from TB (World Health Organization, Global Tuberculosis Control: Surveillance, Planning, Financing. WHO Report 2006, Geneva, Switzerland, ISBN 92-4 156314-1). Co-infection with HIV is driving the increase in incidence (Williams, B. G.; Dye, C. Science, 2003, 301, 1535) and the cause of death in 31% of AIDS patients in Africa can be attributed to TB (Corbett, E. L.; Watt, C. J.; Catherine, J.; Walker, N.; Maher D.; Williams, B. G.; Raviglione, M. C.; Dye, C. Arch. Intl. Med., 2003, 163, 1009, Septkowitz, A.; Raffalli, J.; Riley, T.; Kiehn, T. E.; Armstrong, D. Clin. Microbiol. Rev. 1995, 8, 180). When coupled with the emergence of multi-drug resistant strains of Mycobacterium tuberculosis (MDR-TB), the scale of the problem is amplified. It is now more than a decade since the WHO declared TB “a global health emergency” (World Health Organization, Global Tuberculosis Control: Surveillance, Planning, Financing. WHO Report 2006, Geneva, Switzerland, ISBN 92-4 156314-1).

The limitations of tuberculosis therapy and prevention are well known. The current available vaccine, BCG was introduced in 1921 and fails to protect most people past childhood. Patients who do become infected with active disease currently endure combination therapy with isoniazid, rifampin, pyrazinamide and ethambutol for two months and then continue taking isoniazid and rifampin for a further four months. Daily dosing is required and poor compliance drives the emergence and spread of multi-drug-resistant strains, which are challenging to treat. A recently published detailed review discusses many aspects of TB such as pathogenesis, epidemiology, drug discovery and vaccine development to date (Nature Medicine, Vol 13(3), pages 263-312).

Shorter courses of more active agents which can be taken less frequently and which present a high barrier to the emergence of resistance, i.e. agents which are effective against multi-drug resistant strains of TB (MDR-TB), are urgently required. There is therefore a need to discover and develop new chemical entities to treat TB (recent synthetic leads are reviewed in: Ballell, L.; Field, R. A.; Duncan, K.; Young, R. J. Antimicrob. Agents Chemother. 2005, 49, 2153).

### DETAILED DESCRIPTION OF THE INVENTION

The present invention provides a compound of Formula (I) or a pharmaceutically acceptable salt, solvate or N-oxide thereof:

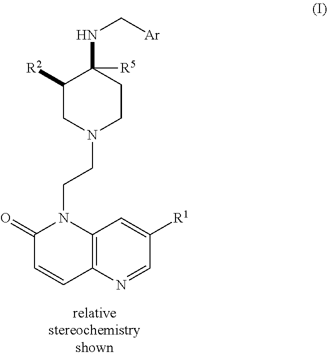

Wherein in Formula (I):

R¹ represents hydrogen; halo; or C₁₋₃alkoxy-;

R² represents hydrogen or hydroxy;

Ar represents a group selected from: phenyl, pyridyl, pyridazinyl, pyrimidinyl, pyrazinyl, thiazolyl, furanyl, imidazolyl and thiophenyl;
wherein
Ar is substituted by a first group R³, wherein R³ represents a group selected from: halo, CF₃, C₁₋₅ alkyl, C₁₋₅ hydroxyalkyl, nitro and cyano; when Ar represents pyridyl, R³ alternatively represents C₁₋₃alkoxy-;
Ar is optionally substituted by a second group R⁴;
when R³ represents halo, the optional R⁴ group represents halo;
when R³ represents CF₃, the optional R⁴ group represents halo;
when R³ represents C₁₋₃alkyl or C₁₋₅ hydroxyalkyl, the optional R⁴ group is selected from halo, CF₃, C₁₋₃alkyl, nitro and C₁₋₃alkoxy-;
when R³ represents nitro, the optional R⁴ group is selected from halo and CF₃;
when R³ represents cyano, the optional R⁴ group is selected from halo, CF₃, C₁₋₃alkyl and nitro;
when R³ represents C₁₋₃alkoxy-, the optional R⁴ group is selected from halo and nitro;
if R² is hydrogen then R⁵ is hydrogen or C₁₋₃ alkyl, and if R² is hydroxyl then R⁵ is hydrogen.

In one embodiment compounds of Formula (I) have the formula (IA), or a pharmaceutically acceptable salt, solvate or N-oxide thereof:

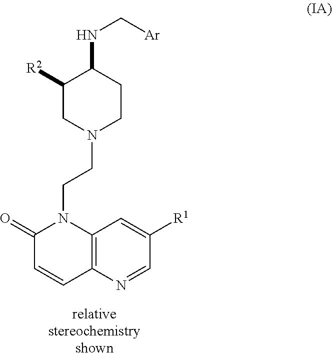

Wherein in Formula (IA):

R¹ represents hydrogen; halo; or C₁₋₃alkoxy-;

R² represents hydrogen or hydroxy;

Ar represents a group selected from: phenyl, pyridyl, pyridazinyl, pyrimidinyl, pyrazinyl, thiazolyl, furanyl, imidazolyl and thiophenyl;
wherein
Ar is substituted by a first group R³, wherein R³ represents a group selected from:
halo, CF₃, C₁₋₃alkyl, nitro and cyano; when represents pyridyl, R³ alternatively represents C₁₋₃alkoxy-;
Ar is optionally substituted by a second group R⁴;
when R³ represents halo, the optional R⁴ group represents halo;
when R³ represents CF₃, the optional R⁴ group represents halo;
when R³ represents C₁₋₃alkyl, the optional R⁴ group is selected from halo, CF₃, C₁₋₃alkyl, nitro and C₁₋₃alkoxy-;
when R³ represents nitro, the optional R⁴ group is selected from halo and CF₃;
when R₃ represents cyano, the optional R⁴ group is selected from halo, CF₃, C₁₋₃alkyl and nitro;
when R³ represents C₁₋₃alkoxy-, the optional R⁴ group is selected from halo and nitro.

The invention further provides a pharmaceutical composition comprising a compound of Formula (I), or a pharmaceutically acceptable salt, solvate or N-oxide thereof, and one or more pharmaceutically acceptable carriers, excipients or diluents.

The invention also provides a method of treatment of tuberculosis in mammals, particularly in man, which method comprises the administration to a mammal in need of such treatment an effective amount of a compound of Formula (I), or a pharmaceutically acceptable salt, solvate or N-oxide thereof.

This invention further provides a method of treatment of bacterial infections in mammals, particularly in man, which method comprises the administration to a mammal in need of such treatment an effective amount of a compound of formula (I), or a pharmaceutically acceptable salt, solvate or N-oxide thereof.

The invention further provides a compound of Formula (I), or a pharmaceutically acceptable salt, solvate or N-oxide thereof, for use in therapy.

The invention yet further provides a compound of Formula (I), or a pharmaceutically acceptable salt, solvate or N-oxide thereof, for use in the treatment of tuberculosis in mammals, particularly in man.

The invention yet further provides a compound of Formula (I), or a pharmaceutically acceptable salt, solvate or N-oxide thereof, for use in the treatment of bacterial infections in mammals, particularly in man.

The invention still further provides the use of a compound of Formula (I), or a pharmaceutically acceptable salt, solvate or N-oxide thereof, in the manufacture of a medicament for use in the treatment of tuberculosis in mammals, particularly in man.

The invention still further provides the use of a compound of Formula (I), or a pharmaceutically acceptable salt, solvate or N-oxide thereof, in the manufacture of a medicament for use in the treatment of bacterial infections in mammals, particularly in man.

The invention also provides a pharmaceutical composition comprising a compound of Formula (I), or a pharmaceutically acceptable salt, solvate or N-oxide thereof, and one or more pharmaceutically acceptable carriers, excipients or diluents, for use in the treatment of tuberculosis in mammals, particularly in man.

The invention also provides a pharmaceutical composition comprising a compound of Formula (I), or a pharmaceutically acceptable salt, solvate or N-oxide thereof, and one or more pharmaceutically acceptable carriers, excipients or diluents, for use in the treatment of bacterial infections in mammals, particularly in man.

In one aspect of the invention, when Ar represents phenyl, the substituent R³ and optional substituent R⁴ occur in the meta- or para- positions relative to the point of attachment of Ar to the remainder of the molecule.

In one aspect of the invention, when R² represents hydroxy, the absolute stereochemistry of the compound of Formula (I), or a pharmaceutically acceptable salt, solvate or N-oxide thereof, is

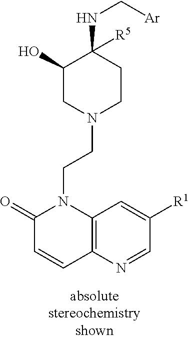

In one aspect of the invention, Ar represents a group selected from: phenyl, pyridyl, pyridazinyl, pyrazinyl, thiazolyl, furanyl and thiophenyl.

In one aspect of the invention, wh1en R³ represents halo, the optional R⁴ group represents halo;
when R³ represents CF₃, the optional R⁴ group represents halo;
when R³ represents C₁₋₃alkyl or C₁₋₅ hydroxyalkyl, the optional R⁴ group is selected from halo, C₁₋₃alkyl and nitro;
when R³ represents C₁₋₃alkoxy-, the optional R⁴ group is selected from halo and nitro.
Examples of alkyl groups R³ are methyl, ethyl and tertiary butyl.
An example of a C₁₋₅ hydroxyalkyl group is hydroxymethyl.

In one aspect of the invention, the substituent R⁴ is present. In another aspect of the invention, the substituent R³ is halo, for example chloro, and the substituent R⁴ is present and is halo, for example chloro.

In an embodiment of the invention Ar is phenyl, R³ is CF₃, and R⁴ is absent, or is present and is methyl or chloro.

In another embodiment Ar is phenyl, R³ is chloro and R⁴ is absent or is present and is chloro.

In another embodiment Ar is pyridyl, R³ is CF₃, and R⁴ is absent.

In another embodiment Ar is pyridyl, R³ is chloro, and R⁴ is present and is methyl, methoxy or fluoro.

In another embodiment Ar is thienyl, R³ is bromo, and R⁴ is present and is bromo or methyl.

In one aspect, compounds which are useful in the present invention are the pharmaceutically acceptable salts of a compound of Formula (I).

In one aspect, compounds which are useful in the present invention include those mentioned in the examples and their pharmaceutically acceptable salts, solvates or N-oxides.

In another aspect, compounds which are useful in the present invention include:

1-[2-(4-{[(5-bromo-2-pyridinyl)methyl]amino}-1-piperidinyl)ethyl]-7-fluoro-1,5-naphthyridin-2(1H)-one;

1-[2-(4-{[(5-bromo-2-pyridinyl)methyl]amino}-1-piperidinyl)ethyl]-7-fluoro-1,5-naphthyridin-2(1H)-one dihydrochloride;

1-[2-(4-{[(2-bromo-1,3-thiazol-5-yl)methyl]amino}-1-piperidinyl)ethyl]-7-(methyloxy)-1,5-naphthyridin-2(1H)-one;

1-[2-(4-{[(2-bromo-1,3-thiazol-5-yl)methyl]amino}-1-piperidinyl)ethyl]-7-(methyloxy)-1,5-naphthyridin-2(1H)-one hydrochloride;

1-[2-(4-{[(3,4-dichlorophenyl)methyl]amino}-1-piperidinyl)ethyl]-7-fluoro-1,5-naphthyridin-2(1H)-one;

1-[2-(4-{[(3,4-dichlorophenyl)methyl]amino}-1-piperidinyl)ethyl]-7-fluoro-1,5-naphthyridin-2(1H)-one;

7-(methyloxy)-1-{2-[4-({[6-(trifluoromethyl)-3-pyridinyl]methyl}amino)-1-piperidinyl]ethyl}-amino)-1-piperidinyl]ethyl}-1,5-naphthyridin-2(1H)-one;

7-(methyloxy)-1-{2-[4-({[6-(trifluoromethyl)-3-pyridinyl]methyl}amino)-1-piperidinyl]ethyl}-1,5-naphthyridin-2(1H)-one hydrochloride;

1-[2-(4-{[(4-fluoro-3-methylphenyl)methyl]amino}-1-piperidinyl)ethyl]-7-(methyloxy)-1,5-naphthyridin-2(1H)-one;

1-[2-(4-{[(4,5-dimethyl-1,3-thiazol-2-yl)methyl]amino}-1-piperidinyl)ethyl]-7-(methyloxy)-1,5-naphthyridin-2(1H)-one;

1-[2-(4-{[(4,5-dichloro-1,3-thiazol-2-yl)methyl]amino}-1-piperidinyl)ethyl]-7-(methyloxy)-1,5-naphthyridin-2(1H)-one;

1-[2-(4-{[(3,4-dichlorophenyl)methyl]amino}-1-piperidinyl)ethyl]-1,5-naphthyridin-2(1H)-one; 1-[2-(4-{[(3,4-dichlorophenyl)methyl]amino}-1-piperidinyl)ethyl]-1,5-naphthyridin-2(1H)-one dihydrochloride;

1-[2-(4-{[(6-bromo-3-pyridinyl)methyl]amino}-1-piperidinyl)ethyl]-1,5-naphthyridin-2(1H)-one;

7-fluoro-1-{2-[4-({[6-(trifluoromethyl)-3-pyridinyl]methyl}amino)-1-piperidinyl]ethyl}-1,5-naphthyridin-2(1H)-one;

1-[2-(4-{[(3,4-dichlorophenyl)methyl]amino}-1-piperidinyl)ethyl]-7-(methyloxy)-1,5-naphthyridin-2(1H)-one;

1-[2-(4-{[(3,4-dichlorophenyl)methyl]amino}-1-piperidinyl)ethyl]-7-(methyloxy)-1,5-naphthyridin-2(1H)-one hydrochloride;

1-[2-(4-{[(3,4-dichlorophenyl)methyl]amino}-1-piperidinyl)ethyl]-7-(methyloxy)-1,5-naphthyridin-2(1H)-one dihydrochloride;

1-[2-(4-{[(4-chlorophenyl)methyl]amino}-1-piperidinyl)ethyl]-7-(methyloxy)-1,5-naphthyridin-2(1H)-one;

1-[2-(4-{[(4-chlorophenyl)methyl]amino}-1-piperidinyl)ethyl]-7-(methyloxy)-1,5-naphthyridin-2(1H)-one dihydrochloride;

1-[2-(4-{[(4-chlorophenyl)methyl]amino}-1-piperidinyl)ethyl]-7-(methyloxy)-1,5-naphthyridin-2(1H)-one hydrochloride;

1-[2-(4-{[(6-bromo-3-pyridinyl)methyl]amino}-1-piperidinyl)ethyl]-7-fluoro-1,5-naphthyridin-2(1H)-one;

7-fluoro-1-[2-(4-{[(4-fluorophenyl)methyl]amino}-1-piperidinyl)ethyl]-1,5-naphthyridin-2(1H)-one;

7-(methyloxy)-1-{2-[4-({[6-(trifluoromethyl)-3-pyridazinyl]methyl}amino)-1-piperidinyl]ethyl}-1,5-naphthyridin-2(1H)-one;

1-[2-(4-{[(5-Bromo-3-isothiazolyl)methyl]amino}-1-piperidinyl)ethyl]-7-(methyloxy)-1,5-naphthyridin-2(1H)-one;

1-[2-(4-{[(4-chlorophenyl)methyl]amino}-1-piperidinyl)ethyl]-7-(ethyloxy)-1,5-naphthyridin-2(1H)-one;

7-(ethyloxy)-1-{2-[4-({[6-(trifluoromethyl)-3-pyridinyl]methyl}amino)-1-piperidinyl]ethyl}-1,5-naphthyridin-2(1H)-one;

1-[2-(4-{[(3,4-dichlorophenyl)methyl]amino}-1-piperidinyl)ethyl]-7-(ethyloxy)-1,5-naphthyridin-2(1H)-one;

1-[2-(4-{[(5-chloro-6-methyl-3-pyridinyl)methyl]amino}-1-piperidinyl)ethyl]-7-fluoro-1,5-naphthyridin-2(1H)-one;

1-[2-(4-{[(4-bromo-2-pyridinyl)methyl]amino}-1-piperidinyl)ethyl]-7-(methyloxy)-1,5-naphthyridin-2(1H)-one;

1-[2-(4-{[(5-bromo-3-pyridinyl)methyl]amino}-1-piperidinyl)ethyl]-7-(methyloxy)-1,5-naphthyridin-2(1H)-one;

1-[2-(4-{[(5-chloro-6-methyl-3-pyridinyl)methyl]amino}-1-piperidinyl)ethyl]-7-(methyloxy)-1,5-naphthyridin-2(1H)-one;

1-[2-(4-{[(4-chloro-3-methylphenyl)methyl]amino}-1-piperidinyl)ethyl]-7-(methyloxy)-1,5-naphthyridin-2(1H)-one;

1-[2-(4-{[(3-chloro-4-methylphenyl)methyl]amino}-1-piperidinyl)ethyl]-1,5-naphthyridin-2(1H)-one;

1-[2-((3R,4S)-4-{[(3,4-dichlorophenyl)methyl]amino}-3-hydroxy-1-piperidinyl)ethyl]-1,5-naphthyridin-2(1H)-one;

1-[2-(4-{[(3,4-dimethylphenyl)methyl]amino}-1-piperidinyl)ethyl]-7-fluoro-1,5-naphthyridin-2(1H)-one;

1-[2-(4-{[(4-chloro-3-methylphenyl)methyl]amino}-1-piperidinyl)ethyl]-1,5-naphthyridin-2(1H)-one;

1-[2-(4-{[(5-chloro-6-methyl-3-pyridinyl)methyl]amino}-1-piperidinyl)ethyl]-1,5-naphthyridin-2(1H)-one;

1-[2-(4-{[(5-bromo-6-methyl-2-pyridinyl)methyl]amino}-1-piperidinyl)ethyl]-7-fluoro-1,5-naphthyridin-2(1H)-one;

1-[2-(4-{[(3-fluoro-4-methylphenyl)methyl]amino}-1-piperidinyl)ethyl]-1,5-naphthyridin-2(1H)-one;

1-[2-(4-{[(3,4-dimethylphenyl)methyl]amino}-1-piperidinyl)ethyl]-1,5-naphthyridin-2(1H)-one;

1-[2-(4-{[(4-chloro-5-fluoro-2-pyridinyl)methyl]amino}-1-piperidinyl)ethyl]-7-fluoro-1,5-naphthyridin-2(1H)-one;

1-[2-(4-{[(5-bromo-2-furanyl)methyl]amino}-1-piperidinyl)ethyl]-7-fluoro-1,5-naphthyridin-2(1H)-one;

1-{2-[4-({[6-(trifluoromethyl)-3-pyridinyl]methyl}amino)-1-piperidinyl]ethyl}-1,5-naphthyridin-2(1H)-one;

1-{2-[4-({[4-chloro-3-(trifluoromethyl)phenyl]methyl}amino)-1-piperidinyl]ethyl}-1,5-naphthyridin-2(1H)-one;

7-(methyloxy)-1-[2-(4-{[(4-nitrophenyl)methyl]amino}-1-piperidinyl)ethyl]-1,5-naphthyridin-2(1H)-one;

1-[2-(4-{[(2,5-dichlorophenyl)methyl]amino}-1-piperidinyl)ethyl]-7-fluoro-1,5-naphthyridin-2(1H)-one;

1-[2-(4-{[(6-bromo-3-pyridinyl)methyl]amino}-1-piperidinyl)ethyl]-7-(methyloxy)-1,5-naphthyridin-2(1H)-one;

1-[2-(4-{[(3,5-dichlorophenyl)methyl]amino}-1-piperidinyl)ethyl]-7-fluoro-1,5-naphthyridin-2(1H)-one;

1-[2-(4-{[(4-fluorophenyl)methyl]amino}-1-piperidinyl)ethyl]-7-(methyloxy)-1,5-naphthyridin-2(1H)-one;

1-{2-[4-({[4-(trifluoromethyl)phenyl]methyl}amino)-1-piperidinyl]ethyl}-1,5-naphthyridin-2(1H)-one;

1-[2-(4-{[(4-chlorophenyl)methyl]amino}-1-piperidinyl)ethyl]-1,5-naphthyridin-2(1H)-one;

1-[2-(4-{[(5-chloro-2-thienyl)methyl]amino}-1-piperidinyl)ethyl]-7-fluoro-1,5-naphthyridin-2(1H)-one;

1-{2-[4-({[5-chloro-6-(methyloxy)-3-pyridinyl]methyl}amino)-1-piperidinyl]ethyl}-7-fluoro-1,5-naphthyridin-2(1H)-one;

1-[2-(4-{[(3,5-dichlorophenyl)methyl]amino}-1-piperidinyl)ethyl]-7-(methyloxy)-1,5-naphthyridin-2(1H)-one;

1-{2-[4-({[5-chloro-6-(methyloxy)-3-pyridinyl]methyl}amino)-1-piperidinyl]ethyl}-7-(methyloxy)-1,5-naphthyridin-2(1H)-one;

1-[2-(4-{[(5-bromo-2-thienyl)methyl]amino}-1-piperidinyl)ethyl]-1,5-naphthyridin-2(1H)-one;

1-[2-(4-{[(2-bromo-1,3-thiazol-5-yl)methyl]amino}-1-piperidinyl)ethyl]-1,5-naphthyridin-2(1H)-one;

1-[2-(4-{[(4,5-dibromo-2-thienyl)methyl]amino}-1-piperidinyl)ethyl]-7-(methyloxy)-1,5-naphthyridin-2(1H)-one;

1-{2-[4-({[3-chloro-4-(methyloxy)phenyl]methyl}amino)-1-piperidinyl]ethyl}-1,5-naphthyridin-2(1H)-one;

1-[2-(4-{[(3,4-dibromophenyl)methyl]amino}-1-piperidinyl)ethyl]-1,5-naphthyridin-2(1H)-one;

4-{[(1-{2-[7-(methyloxy)-2-oxo-1,5-naphthyridin-1(2H)-yl]ethyl}-4-piperidinyl)amino]methyl}benzonitrile;

1-[2-(4-{[(5-bromo-2-thienyl)methyl]amino}-1-piperidinyl)ethyl]-7-(methyloxy)-1,5-naphthyridin-2(1H)-one;

1-[2-(4-{[(4,5-dibromo-2-thienyl)methyl]amino}-1-piperidinyl)ethyl]-1,5-naphthyridin-2(1H)-one;

1-[2-(4-{[(5-bromo-4-methyl-2-thienyl)methyl]amino}-1-piperidinyl)ethyl]-7-fluoro-1,5-naphthyridin-2(1H)-one;

1-[2-(4-{[(3-chlorophenyl)methyl]amino}-1-piperidinyl)ethyl]-1,5-naphthyridin-2(1H)-one;

1-[2-(4-{[(3,5-dichlorophenyl)methyl]amino}-1-piperidinyl)ethyl]-1,5-naphthyridin-2(1H)-one;

7-fluoro-1-[2-(4-{[(4-methyl-3-nitrophenyl)methyl]amino}-1-piperidinyl)ethyl]-1,5-naphthyridin-2(1H)-one;

1-[2-(4-{[(4-bromo-1,3-thiazol-2-yl)methyl]amino}-1-piperidinyl)ethyl]-7-fluoro-1,5-naphthyridin-2(1H)-one;

1-[2-(4-{[(3-chloro-4-methylphenyl)methyl]amino}-1-piperidinyl)ethyl]-7-fluoro-1,5-naphthyridin-2(1H)-one;

7-fluoro-1-{2-[4-({[5-fluoro-6-(methyloxy)-3-pyridinyl]methyl}amino)-1-piperidinyl]ethyl}-1,5-naphthyridin-2(1H)-one;

1-[2-(4-{[(5-bromo-2-furanyl)methyl]amino}-1-piperidinyl)ethyl]-7-(methyloxy)-1,5-naphthyridin-2(1H)-one;

1-[2-(4-{[(4-bromo-1,3-thiazol-2-yl)methyl]amino}-1-piperidinyl)ethyl]-7-(methyloxy)-1,5-naphthyridin-2(1H)-one;

1-[2-(4-{[(5-bromo-4-methyl-2-thienyl)methyl]amino}-1-piperidinyl)ethyl]-1,5-naphthyridin-2(1H)-one;

1-{2-[4-({[5-fluoro-6-(methyloxy)-3-pyridinyl]methyl}amino)-1-piperidinyl]ethyl}-7-(methyloxy)-1,5-naphthyridin-2(1H)-one;

7-(methyloxy)-1-{2-[4-({[6-(methyloxy)-3-pyridinyl]methyl}amino)-1-piperidinyl]ethyl}-1,5-naphthyridin-2(1H)-one;

1-[2-(4-{[(5-fluoro-6-methyl-2-pyridinyl)methyl]amino}-1-piperidinyl)ethyl]-7-(methyloxy)-1,5-naphthyridin-2(1H)-one;

1-[2-(4-{[(3-chloro-4-methylphenyl)methyl]amino}-1-piperidinyl)ethyl]-7-(methyloxy)-1,5-naphthyridin-2(1H)-one;

1-[2-(4-{[(6-fluoro-5-methyl-3-pyridinyl)methyl]amino}-1-piperidinyl)ethyl]-7-(methyloxy)-1,5-naphthyridin-2(1H)-one;

1-[2-(4-{[(4-chloro-5-fluoro-2-pyridinyl)methyl]amino}-1-piperidinyl)ethyl]-7-(methyloxy)-1,5-naphthyridin-2(1H)-one;

1-[2-(4-{[(5-bromo-4-methyl-1,3-thiazol-2-yl)methyl]amino}-1-piperidinyl)ethyl]-1,5-naphthyridin-2(1H)-one;

1-[2-(4-{[(3,5-Dimethylphenyl)methyl]amino}-1-piperidinyl)ethyl]-7-(methyloxy)-1,5-naphthyridin-2(1H)-one;

1-[2-(4-{[(3,4-Dimethylphenyl)methyl]amino}-1-piperidinyl)ethyl]-7-(methyloxy)-1,5-naphthyridin-2(1H)-one;

1-[2-(4-{[(3-fluoro-5-methylphenyl)methyl]amino}-1-piperidinyl)ethyl]-7-(methyloxy)-1,5-naphthyridin-2(1H)-one;

1-[2-(4-{[(4-bromo-3-methylphenyl)methyl]amino}-1-piperidinyl)ethyl]-7-(methyloxy)-1,5-naphthyridin-2(1H)-one;

1-[2-(4-{[(5,6-dichloro-3-pyridinyl)methyl]amino}-1-piperidinyl)ethyl]-7-(methyloxy)-1,5-naphthyridin-2(1H)-one;

1-[2-(4-{[(3-fluoro-4-methylphenyl)methyl]amino}-1-piperidinyl)ethyl]-7-(methyloxy)-1,5-naphthyridin-2(1H)-one;

1-[2-(4-{[(4-chloro-3-methylphenyl)methyl]amino}-1-piperidinyl)ethyl]-7-fluoro-1,5-naphthyridin-2(1H)-one;

1-[2-(4-{[(5-bromo-3-pyridinyl)methyl]amino}-1-piperidinyl)ethyl]-7-fluoro-1,5-naphthyridin-2(1H)-one;

1-[2-(4-{[(4-bromo-2-thienyl)methyl]amino}-1-piperidinyl)ethyl]-1,5-naphthyridin-2(1H)-one;

1-[2-(4-{[(3,4-dibromophenyl)methyl]amino}-1-piperidinyl)ethyl]-7-fluoro-1,5-naphthyridin-2(1H)-one;

1-[2-(4-{[(5-bromo-2-thienyl)methyl]amino}-1-piperidinyl)ethyl]-7-fluoro-1,5-naphthyridin-2(1H)-one;

7-fluoro-1-{2-[4-({[4-(trifluoromethyl)phenyl]methyl}amino)-1-piperidinyl]ethyl}-1,5-naphthyridin-2(1H)-one;

1-[2-(4-{[(3,4-difluorophenyl)methyl]amino}-1-piperidinyl)ethyl]-7-fluoro-1,5-naphthyridin-2(1H)-one;

1-[2-(4-{[(5-bromo-6-methyl-2-pyridinyl)methyl]amino}-1-piperidinyl)ethyl]-7-(methyloxy)-1,5-naphthyridin-2(1H)-one;

1-[2-(4-{[(3,4-dibromophenyl)methyl]amino}-1-piperidinyl)ethyl]-7-(methyloxy)-1,5-naphthyridin-2(1H)-one;

7-(methyloxy)-1-{2-[4-({[4-(trifluoromethyl)phenyl]methyl}amino)-1-piperidinyl]ethyl}-1,5-naphthyridin-2(1H)-one;

1-[2-(4-{[(3,4-difluorophenyl)methyl]amino}-1-piperidinyl)ethyl]-7-(methyloxy)-1,5-naphthyridin-2(1H)-one;

1-[2-(4-{[(3-chlorophenyl)methyl]amino}-1-piperidinyl)ethyl]-7-(methyloxy)-1,5-naphthyridin-2(1H)-one;

1-{2-[4-({[4-chloro-3-(trifluoromethyl)phenyl]methyl}amino)-1-piperidinyl]ethyl}-7-(methyloxy)-1,5-naphthyridin-2(1H)-one;

1-[2-(4-{[(2,4-dichlorophenyl)methyl]amino}-1-piperidinyl)ethyl]-7-(methyloxy)-1,5-naphthyridin-2(1H)-one;

1-[2-(4-{[(5-bromo-2-pyridinyl)methyl]amino}-1-piperidinyl)ethyl]-7-(methyloxy)-1,5-naphthyridin-2(1H)-one;

1-[2-(4-{[(2-chloro-1,3-thiazol-5-yl)methyl]amino}-1-piperidinyl)ethyl]-7-(methyloxy)-1,5-naphthyridin-2(1H)-one;

1-[2-(4-{[(5-bromo-4-methyl-2-thienyl)methyl]amino}-1-piperidinyl)ethyl]-7-(methyloxy)-1,5-naphthyridin-2(1H)-one

1-[2-((3R,4S)-4-{[(3,4-dichlorophenyl)methyl]amino}-3-hydroxy-1-piperidinyl)ethyl]-7-fluoro-1,5-naphthyridin-2(1H)-one;

1-{2-[(3R,4S)-3-hydroxy-4-({[6-(trifluoromethyl)-3-pyridinyl]methyl}amino)-1-piperidinyl]ethyl}-7-(methyloxy)-1,5-naphthyridin-2(1H)-one;

1-[2-((3R,4S)-4-{[(3,4-dichlorophenyl)methyl]amino}-3-hydroxy-1-piperidinyl)ethyl]-7-(methyloxy)-1,5-naphthyridin-2(1H)-one;

1-{2-[(3S,4R)-3-hydroxy-4-({[6-(trifluoromethyl)-3-pyridinyl]methyl}amino)-1-piperidinyl]ethyl}-7-(methyloxy)-1,5-naphthyridin-2(1H)-one;

1-[2-((3S,4R)-4-{[(3,4-dichlorophenyl)methyl]amino}-3-hydroxy-1-piperidinyl)ethyl]-7-(methyloxy)-1,5-naphthyridin-2(1H)-one;

1-{2-[(3R,4S)-3-hydroxy-4-({[6-(trifluoromethyl)-3-pyridinyl]methyl}amino)-1-piperidinyl]ethyl}-1,5-naphthyridin-2(1H)-one;

7-fluoro-1-{2-[(3R,4S)-3-hydroxy-4-({[6-(trifluoromethyl)-3-pyridinyl]methyl}amino)-1-piperidinyl]ethyl}-1,5-naphthyridin-2(1H)-one;

1-[2-(4-{[(3-chlorophenyl)methyl]amino}-1-piperidinyl)ethyl]-7-fluoro-1,5-naphthyridin-2(1H)-one;

1-[2-(4-{[(4-chlorophenyl)methyl]amino}-1-piperidinyl)ethyl]-7-fluoro-1,5-naphthyridin-2(1H)-one;

1-[2-(4-{[(4-bromo-2-thienyl)methyl]amino}-1-piperidinyl)ethyl]-7-(methyloxy)-1,5-naphthyridin-2(1H)-one;

1-[2-(4-{[(5-chloro-2-thienyl)methyl]amino}-1-piperidinyl)ethyl]-7-(methyloxy)-1,5-naphthyridin-2(1H)-one;

1-[2-(4-{[(2-bromo-1,3-thiazol-5-yl)methyl]amino}-1-piperidinyl)ethyl]-7-fluoro-1,5-naphthyridin-2(1H)-one;

1-[2-(4-{[(4,5-dibromo-2-thienyl)methyl]amino}-1-piperidinyl)ethyl]-7-fluoro-1,5-naphthyridin-2(1H)-one;

1-[2-(4-{[(4-bromo-2-thienyl)methyl]amino}-1-piperidinyl)ethyl]-7-fluoro-1,5-naphthyridin-2(1H)-one;

7-(methyloxy)-1-{2-[4-({[5-(methyloxy)-6-nitro-2-pyridinyl]methyl}amino)-1-piperidinyl]ethyl}-1,5-naphthyridin-2(1H)-one;

1-[2-(4-{[(6-chloro-2-pyrazinyl)methyl]amino}-1-piperidinyl)ethyl]-7-(methyloxy)-1,5-naphthyridin-2(1H)-one;

2-fluoro-5-{[(1-{2-[7-(methyloxy)-2-oxo-1,5-naphthyridin-1(2H)-yl]ethyl}-4-piperidinyl)amino]methyl}benzonitrile;

7-(methyloxy)-1-{2-[4-({[2-(trifluoromethyl)-4-pyrimidinyl]methyl}amino)-1-piperidinyl]ethyl}-1,5-naphthyridin-2(1H)-one:

1-[2-(4-{[(4,6-dimethyl-2-pyridinyl)methyl]amino}-1-piperidinyl)ethyl]-7-(methyloxy)-1,5-naphthyridin-2(1H)-one;

1-[2-(4-{[(5-chloro-3-pyridinyl)methyl]amino}-1-piperidinyl)ethyl]-7-(methyloxy)-1,5-naphthyridin-2(1H)-one;

1-[2-(4-{[(5-chloro-2-pyridinyl)methyl]amino}-1-piperidinyl)ethyl]-7-(methyloxy)-1,5-naphthyridin-2(1H)-one;

1-[2-((3R,4S)-4-{[(5-chloro-6-methyl-3-pyridinyl)methyl]amino}-3-hydroxy-1-piperidinyl)ethyl]-7-(methyloxy)-1,5-naphthyridin-2(1H)-one;

7-fluoro-1-[2-(4-{[(2-methyl-1H-imidazol-4-yl)methyl]amino}-1-piperidinyl)ethyl]-1,5-naphthyridin-2(1H)-one trifluoroacetate;

2-fluoro-5-[({1-[2-(7-fluoro-2-oxo-1,5-naphthyridin-1(2H)-yl)ethyl]-4-piperidinyl}amino)methyl]benzonitrile;

1-{2-[4-({[4-(1,1-dimethylethyl)phenyl]methyl}amino)-1-piperidinyl]ethyl}-7-fluoro-1,5-naphthyridin-2(1H)-one;

7-fluoro-1-[2-(4-{[(6-fluoro-5-methyl-3-pyridinyl)methyl]amino}-1-piperidinyl)ethyl]-1,5-naphthyridin-2(1H)-one;

1-[2-((3R,4S)-4-{[(5-chloro-6-methyl-3-pyridinyl)methyl]amino}-3-hydroxy-1-piperidinyl)ethyl]-7-fluoro-1,5-naphthyridin-2(1H)-one hydrochloride;

1-[2-(4-{[(5,6-dimethyl-3-pyridinyl)methyl]amino}-1-piperidinyl)ethyl]-7-fluoro-1,5-naphthyridin-2(1H)-one;

1-[2-(4-{[(5-bromo-6-methyl-3-pyridinyl)methyl]amino}-1-piperidinyl)ethyl]-7-fluoro-1,5-naphthyridin-2(1H)-one;

1-[2-(4-{[(5-chloro-6-ethyl-3-pyridinyl)methyl]amino}-1-piperidinyl)ethyl]-7-fluoro-1,5-naphthyridin-2(1H)-one;

7-fluoro-1-[2-((3R,4S)-4-{[(5-fluoro-6-methyl-3-pyridinyl)methyl]amino}-3-hydroxy-1-piperidinyl)ethyl]-1,5-naphthyridin-2(1H)-one;

1-[2-(4-{[(5,6-dichloro-3-pyridinyl)methyl]amino}-1-piperidinyl)ethyl]-7-fluoro-1,5-naphthyridin-2(1H)-one;

1-[2-((3R,4S)-4-{[(5-bromo-6-methyl-3-pyridinyl)methyl]amino}-3-hydroxy-1-piperidinyl)ethyl]-7-fluoro-1,5-naphthyridin-2(1H)-one;

7-fluoro-1-{2-[4-methyl-4-({[6-(trifluoromethyl)-3-pyridinyl]methyl}amino)-1-piperidinyl]ethyl}-1,5-naphthyridin-2(1H)-one;

1-[2-(4-{[(3,4-dichlorophenyl)methyl]amino}-4-methyl-1-piperidinyl)ethyl]-7-(methyloxy)-1,5-naphthyridin-2(1H)-one;

and,

1-[2-(4-{[(3,4-dichlorophenyl)methyl]amino}-4-methyl-1-piperidinyl)ethyl]-7-fluoro-1,5-naphthyridin-2(1H)-one,

or a pharmaceutically acceptable salt, solvate or N-oxide thereof.

Terms and Definitions

The term “(C₁₋₃)alkyl” as used herein refers to a straight or branched chain alkyl group having 1 to 3 carbon atoms. Examples of (C₁₋₃)alkyl groups include methyl, ethyl, n-propyl, iso-propyl.

The term “halo” as used herein refers to fluoro, chloro, bromo and iodo groups. In one aspect, the term “halo” as used herein refers to fluoro, chloro and bromo groups. In another aspect, the term “halo” as used herein refers to chloro, bromo and iodo groups.

The term “(C₁₋₃)alkoxy” as used herein refers to a straight or branched chain alkoxy group having 1 to 3 carbon atoms. Examples of (C₁₋₃)alkoxy groups include, methoxy, ethoxy, propoxy and isopropoxy.

The term “compounds of the invention” as used herein means a compound of Formula (I) or a pharmaceutically acceptable salt, solvate or N-oxide thereof. The term “a compound of the invention” means any one of the compounds of the invention as defined above.

Furthermore, it will be understood that phrases such as “a compound of Formula (I) or a pharmaceutically acceptable salt, solvate or N-oxide thereof” or “compounds of the invention” are intended to encompass the compound of Formula (I), a pharmaceutically acceptable salt, solvate or N-oxide of the compound of Formula (I), or any pharmaceutically acceptable combination of these. Thus by way of non-limiting example used here for illustrative purpose, “a compound of Formula (I) or a pharmaceutically acceptable salt, solvate or N-oxide thereof” encompasses a pharmaceutically acceptable salt of a compound of Formula (I) which is present as a solvate, or this phrase may include a mixture of a compound of Formula (I) and a salt of a compound of Formula (I).

It will be further appreciated that all crystalline forms, polymorphs and enantiomers of the compounds of the invention, or mixtures thereof, are contemplated to be within the scope of the present invention. Unless otherwise specified (for example when the absolute stereochemistry is shown), for compounds of the invention which possesses stereocentres and which can therefore form enantiomers, (for example, when R² represents hydroxy), the compound contains a 1:1 mixture of enantiomers, i.e. a racemic mixture of enantiomers. These may be separated using conventional techniques such as chiral HPLC.

Some of the compounds of this invention may be crystallised or recrystallised from solvents such as aqueous and organic solvents. In such cases solvates may be formed. This invention includes within its scope stoichiometric solvates including hydrates as well as compounds containing variable amounts of water that may be produced by processes such as lyophilisation.

Since the compounds of Formula (I) are intended for use in pharmaceutical compositions it will readily be understood that in particular embodiments they are provided in substantially pure form, for example at least 60% pure, more suitably at least 75% pure and particularly at least 85%, especially at least 98% pure (% are on a weight for weight basis). Impure preparations of the compounds may be used for preparing the more pure forms used in the pharmaceutical compositions; these less pure preparations of the compounds should contain at least 1%, more suitably at least 5% and more particularly from 10 to 59% of a compound of Formula (I) or pharmaceutically acceptable salt, solvate and/or N-oxide thereof.

Pharmaceutically acceptable salts of the compounds of Formula (I) include the acid addition or quaternary ammonium salts, for example their salts with mineral acids e.g. hydrochloric, hydrobromic, sulphuric nitric or phosphoric acids, or organic acids, e.g. acetic, fumaric, succinic, maleic, citric, benzoic, p-toluenesulphonic, methanesulphonic, naphthalenesulphonic acid or tartaric acids. In one aspect of the invention, the salt of a compound of Formula (I) is the hydrochloride salt. In another aspect, the salt of a compound of Formula (I) is the dihydrochloride salt. Compounds of Formula (I) may also be prepared as the N-oxide. The invention extends to all such salts, solvates and/or N-oxides.

Compound Preparation

The general procedures used to synthesise the compounds of Formula (I), are described in reaction Schemes 1-11 and are illustrated in the Examples.

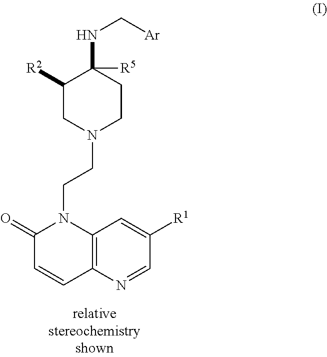

Compounds of Formula (I) and (IA), wherein R¹, R² , R⁵ and Ar are as defined for Formula (I) and (IA), may be prepared by a reductive amination reaction between a compound of Formula (II), wherein R¹ , R² and R⁵ are as defined for Formula (I), or an acid salt of a compound of Formula (II) such as a hydrochloride salt, and an aldehyde of Formula (III), wherein Ar is as defined for Formula (I), according to Scheme 1. Compounds (II) are reacted with compounds (III) in the presence of a hydride donor such as NaBH(AcO)₃ or polymer-supported NaBH₃CN, optionally in the presence of a catalytic acid such as acetic acid or a base such as triethylamine, in the presence of a suitable solvent such as 1,2-dicloroethane, or THF, or a mixture of DCM and MeOH, to give compounds (I).

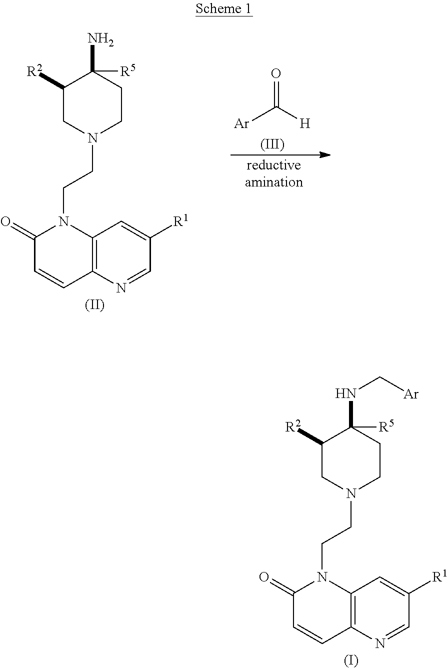

Alternatively, compounds of Formula (I), wherein R¹ , R⁵ and Ar are as defined for Formula (I) and R² is hydrogen, may be prepared by an alkylation reaction between a compound of Formula (II), wherein R¹ is as defined for Formula (I) and R² is hydrogen, and an alkylating agent of Formula (IV), wherein hal is a halo group, for example bromo, according to Scheme 2. Compounds (II) are reacted with compounds (IV) in the presence of a suitable base such as K₂CO₃ in a suitable solvent such as acetonitrile, to give compounds (I).

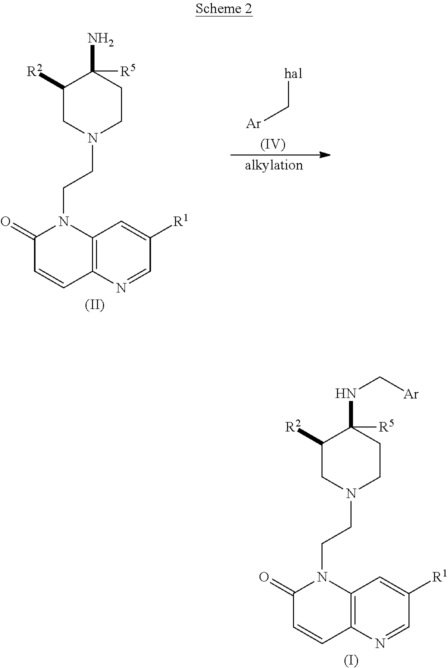

The acid salts of compounds of Formula (I), wherein R¹, R², R⁵ and Ar are as defined for Formula (I), may be prepared by treating a solution of a compound of Formula (I) in a suitable solvent such as DCM with a suitable acid. For example, to make a hydrochloride salt of a compound of Formula (I), a solution of HCl in 1,4-dioxane or in MeOH may be employed. If a monohydrochloride salt of a compound of Formula (I) is required, for example one equivalent of HCl may be used. If a dihydrochloride salt of a compound of Formula (I) is required, an excess of HCl may be used.

Compounds of Formula (II), wherein R² is as defined for Formula (I) and R¹ is hydrogen; halo; or C₁₋₃alkoxy-, may be prepared by a reductive amination reaction between compounds of Formula (V), wherein R¹ is hydrogen; halo; or C₁₋₃alkoxy-, and compounds of Formula (VI), wherein R² is as defined for Formula (I) and P is a nitrogen protecting group such as BOC, followed by a deprotection reaction, according to Scheme 3. Compounds (V) are reacted with compounds (VI) in the presence of a hydride donor such as NaBH(AcO)₃, in a suitable solvent such as CHCl₃ or DCM, or a mixture of either of these solvents with MeOH, followed by a deprotection reaction to remove the protecting group P. For Example, when the protecting group is BOC, this may be removed by treatment with a suitable acid such as HCl (for example a solution in 1,4-dioxane) in a suitable solvent such as DCM, to give the hydrochloride salt of compounds (II). If the free base of compound (II) is required, this may be followed by treatment with a suitable base such as aqueous NaOH with colling, to give compounds (II).

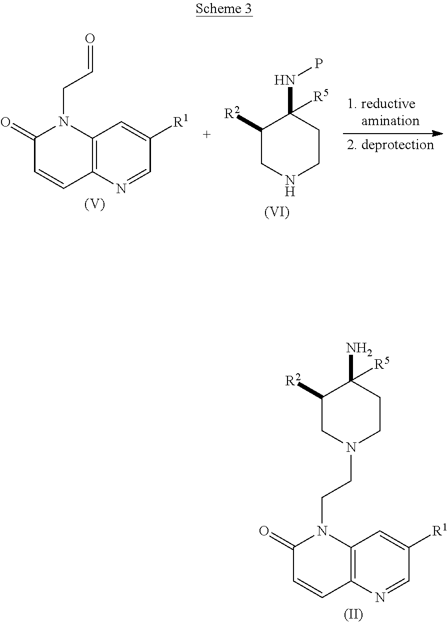

Compounds of Formula (II), wherein R² is hydrogen and R¹ is C₁₋₃alkoxy-, may be prepared from a compound of Formula (II), wherein R² is hydrogen and R¹ is fluoro, as shown in Scheme 4. The fluoro compound of Formula (II) may be treated with a suitable base such as NaH in a suitable solvent such as a mixture of 1,4-dioxane and the appropriate alcohol C₁₋₃alkylOH, in the presence of heat, for example in a microwave oven.

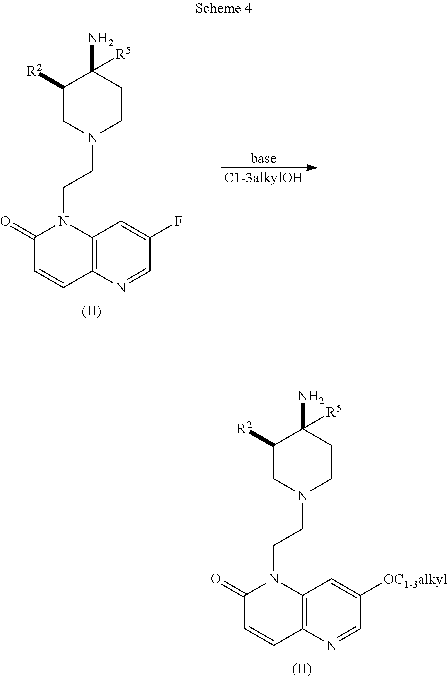

Compounds of Formula (VI), wherein R² is hydrogen are commercially available (for example from Aldrich). Compounds of Formula (VI), wherein R² is hydroxy and protecting group P is for example BOC, may be prepared according to the procedure given in WO2004058144, Example 5(c), cis-(3-hydroxy-piperidin-4-yl)-carbamic acid tert-butyl ester Enantiomer 1.

Compounds of Formula (V), wherein R¹ is hydrogen; halo; or C₁₋₃alkoxy-, may be prepared by oxidation of a compound of Formula (VII), wherein R¹ is hydrogen; halo; or C₁₋₃alkoxy-, according to Scheme 5. Compounds (VII) are treated with a suitable oxidising agent such as a mixture of sodium periodate and osmium tetroxide, in a suitable solvent such as a mixture of 1,4-dioxane and water, to give compounds (V).

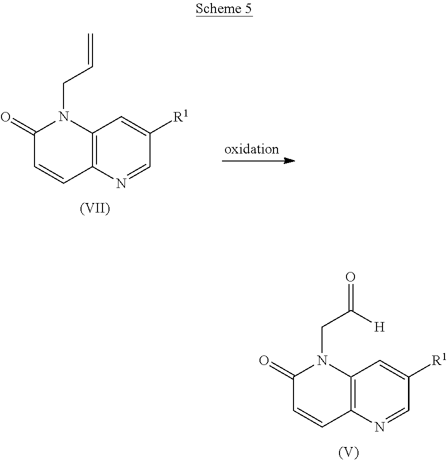

Compounds of Formula (VII), wherein R¹ is hydrogen; halo; or C₁₋₃alkoxy-, may be prepared by N-alkylation of compounds of Formula (VIII), wherein R¹ is hydrogen; halo; or C₁₋₃alkoxy-, according to Scheme 6. Compounds (VIII) are treated with a compound of Formula CH₂═CH—CH₂-hal, wherein hal is a halo group, for example with allyl bromide, in the presence of a suitable base such as NaH and optionally a catalyst such as LiBr, in a suitable solvent such as a mixture of DME and DMF, at elevated temperature, for example from 50-75° C., to give compounds (VIII).

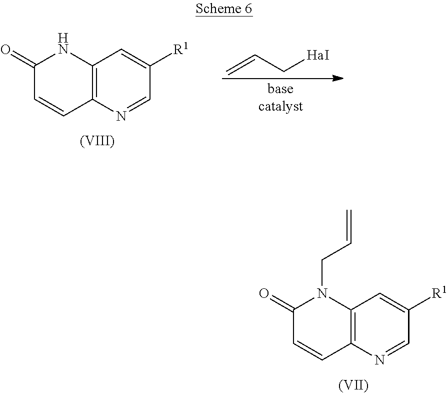

Compounds of Formula (VIII), wherein R¹ is hydrogen; halo; or C₁₋₃alkoxy-, may be prepared by a hydrolysis reaction of compounds of Formula (IX), wherein R¹ is hydrogen; halo; or C₁₋₃alkoxy-, according to Scheme 7. Compounds (IX) may be treated with an acid, such as aqueous HCl, at elevated temperature, for example 90-110° C., to give compounds (VIII).

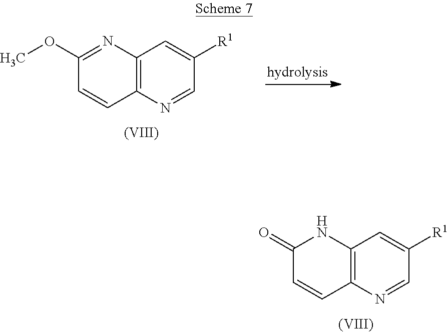

Compounds of Formula (IX), wherein R¹ is hydrogen; halo; or C₁₋₃alkoxy-, may be prepared from compounds of Formula (X), wherein R¹ is hydrogen; halo; or C₁₋₃alkoxy-, and halo is chloro or bromo, for example bromo, according to Scheme 8. Compounds (X) are subjected to hydrogenation in the presence of a suitable catalyst such as palladium on charcoal, in the presence of a suitable base such as NaHCO₃, to give compounds (IX).

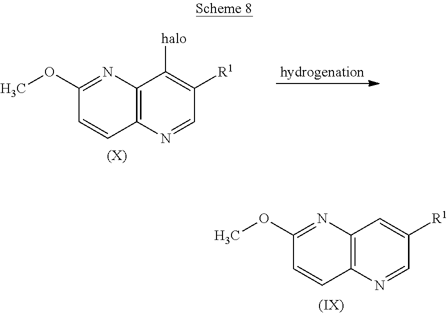

Compounds of Formula (X), wherein R¹ is hydrogen or halo, may be prepared from compounds of Formula (XI), wherein R¹ is hydrogen or halo, according to Scheme 9. Compounds (XI) are treated with a source of chloride or bromide, such as PBr₃, in a suitable solvent such as DMF, to aive compounds (X).

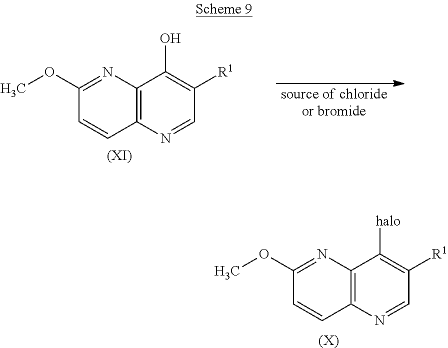

The compound of Formula (XI), wherein R¹ is hydrogen, may be prepared according to the procedure provided in WO2007016610, preparation 2 (a).

Compounds of Formula (XI), wherein R¹ is halo, for example fluoro, may be prepared according to the synthesis in Scheme 10.

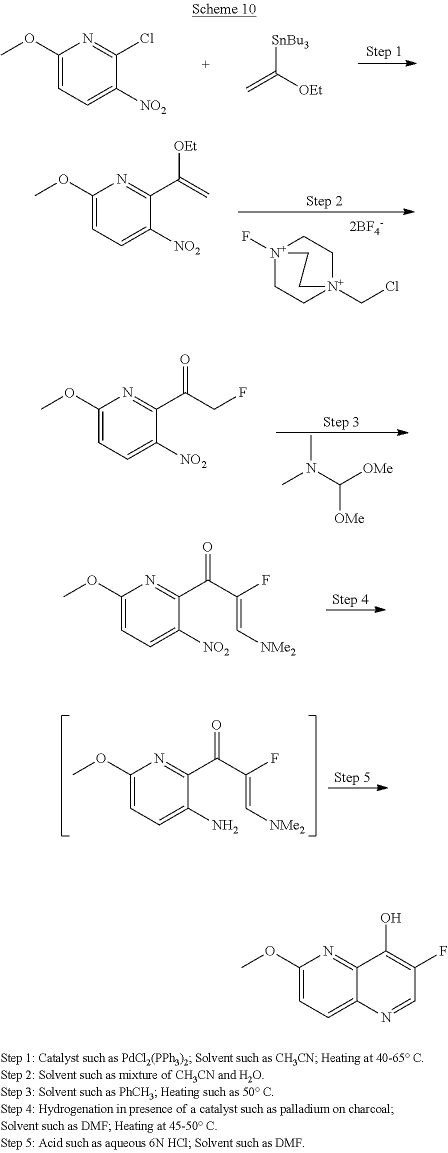

Compounds of Formula (X), wherein R¹ is C₁₋₃alkoxy-, may be prepared from compounds of Formula (X), wherein R¹ is halo, for example fluoro, according to Scheme 11. Compounds (X), wherein R¹ is fluoro, are treated with an appropriate base such as NaOC₁₋₃alkyl, for example NaOMe, in the appropriate alcohol solvent (C₁₋₃alkyl0H), for example methanol, at elevated temperature, for example 40-65° C.

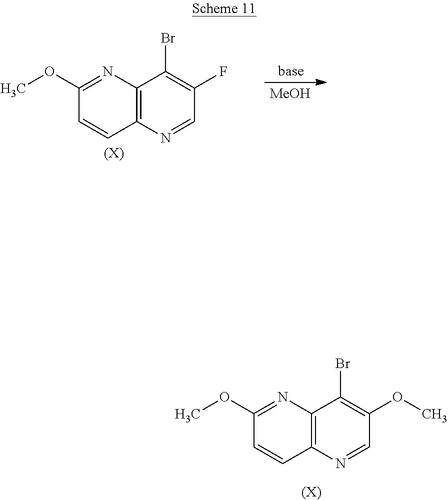

Compounds of Formula (I) in which R⁵ is methyl may be prepared using Scheme 12 below. Although illustrated with a group Ar which is pyridyl, the same methodology is expected to be useful for other groups Ar.

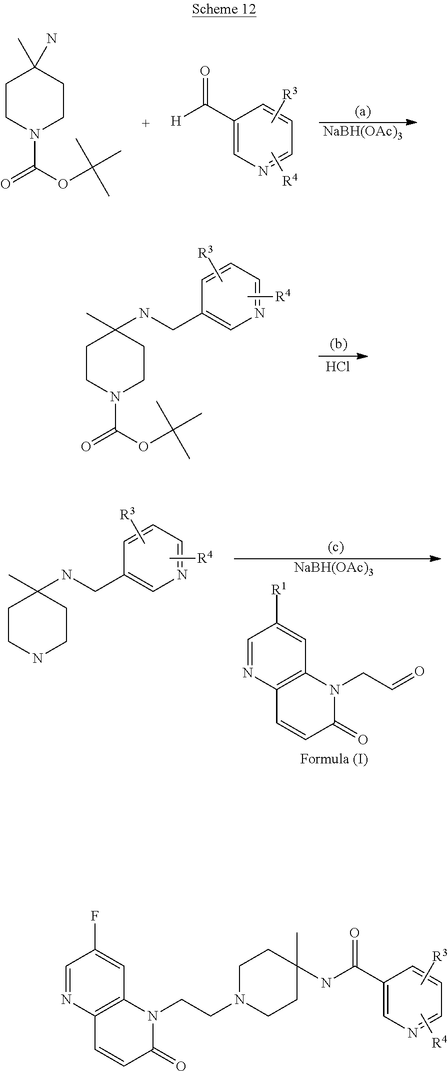

Step 12(a) may be performed at room temperature in dichloromethane. Step 12(b) may be performed in dichloromethane at room temperature. Step 12(c) may be performed at room temperature in dichloromethane.

Those skilled in the art will appreciate that in the preparation of the compound of Formula (I), it may be necessary and/or desirable to protect one or more sensitive groups in the molecule or the appropriate intermediate to prevent undesirable side reactions. Suitable protecting groups for use according to the present invention are well known to those skilled in the art and may be used in a conventional manner. See, for example, “Protective groups in organic synthesis” by T. W. Greene and P. G. M. Wuts (John Wiley & sons 1991) or “Protecting Groups” by P.J. Kocienski (Georg Thieme Verlag 1994). Examples of suitable amino protecting groups include acyl type protecting groups (e.g. formyl, trifluoroacetyl, acetyl), aromatic urethane type protecting groups (e.g. benzyloxycarbonyl (Cbz) and substituted Cbz), aliphatic urethane protecting groups (e.g. 9-fluorenylmethoxycarbonyl (Fmoc), t-butyloxycarbonyl (Boc), isopropyloxycarbonyl, cyclohexyloxycarbonyl) and alkyl or aralkyl type protecting groups (e.g. benzyl, trityl, chlorotrityl). Examples of suitable oxygen protecting groups may include for example alky silyl groups, such as trimethylsilyl or tert-butyldimethylsilyl; alkyl ethers such as tetrahydropyranyl or tert-butyl; or esters such as acetate.

It will be readily apparent to those skilled in the art that other compounds of Formula (I) may be prepared using methods analogous to those outlined above, or by reference to the experimental procedures detailed in the Examples provided herein. Further details for the preparation of compounds of Formula (I) are found in the Examples.

Compositions and Formulations

The compounds of the invention may be formulated for administration in any convenient way for use in human or veterinary medicine, by analogy with formulation of antibacterials, or formulation of other antitubercular agents.

The compounds of the invention will normally, but not necessarily, be formulated into pharmaceutical compositions prior to administration to a patient. In one aspect, the invention is directed to a pharmaceutical composition comprising a compound of Formula (I), or a pharmaceutically acceptable salt, solvate or N-oxide thereof. In another aspect the invention is directed to a pharmaceutical composition comprising a compound of Formula (I), or a pharmaceutically acceptable salt, solvate or N-oxide thereof, and one or more pharmaceutically acceptable carriers, excipients or diluents. The carrier, excipient or diluent must be “acceptable” in the sense of being compatible with the other ingredients of the formulation and not deleterious to the recipient thereof.

The pharmaceutical compositions of the invention include those in a form adapted for oral, or parenteral use and may be used for the treatment of tuberculosis in mammals including humans.

The pharmaceutical compositions of the invention include those in a form adapted for oral, topical or parenteral use and may be used for the treatment of bacterial infections in mammals including humans.

The composition may be formulated for administration by any convenient route. For the treatment of tuberculosis, the compositions may be in the form of tablets, capsules, powders, granules, lozenges, aerosols or liquid preparations, such as oral or sterile parenteral solutions or suspensions. For the treatment of bacterial infections the compositions may be in the form of tablets, capsules, powders, granules, lozenges, creams, aerosols or liquid preparations, such as oral or sterile parenteral solutions or suspensions.

For the treatment of bacterial infections the topical formulations of the present invention may be presented as, for instance, ointments, creams or lotions, eye ointments and eye or ear drops, impregnated dressings and aerosols, and may contain appropriate conventional additives such as preservatives, solvents to assist drug penetration and emollients in ointments and creams.

The formulations may also contain compatible conventional carriers, such as cream or ointment bases and ethanol or ( )eyl alcohol for lotions. Such carriers may be present as from about 1% up to about 98% of the formulation. More usually they will form up to about 80% of the formulation.

Tablets and capsules for oral administration may be in unit dose presentation form, and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavouring or colouring agents.

Suppositories will contain conventional suppository bases, e.g. cocoa-butter or other glyceride.

For parenteral administration, fluid unit dosage forms are prepared utilizing the compound and a sterile vehicle, water being preferred. The compound, depending on the vehicle and concentration used, can be either suspended or dissolved in the vehicle. In preparing solutions the compound can be dissolved in water for injection and filter sterilised before filling into a suitable vial or ampoule and sealing.

In one aspect of the invention, agents such as a local anaesthetic, preservative and buffering agents can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilization cannot be accomplished by filtration. The compound can be sterilised by exposure to ethylene oxide before suspending in the sterile vehicle. Advantageously, a surfactant or wetting agent is included in the composition to facilitate uniform distribution of the compound.

The compositions may contain from 0.1% by weight, preferably from 10-60% by weight, of the active material, depending on the method of administration. Where the compositions comprise dosage units, each unit will preferably contain from 50-1000 mg of the active ingredient. The dosage as employed for adult human treatment will preferably range from 100 to 3000 mg per day, for instance 1500 mg per day depending on the route and frequency of administration. Such a dosage corresponds to 1.5 to 50 mg/kg per day. Suitably the dosage is from 5 to 30 mg/kg per day.

The compound of Formula (I), or a pharmaceutically acceptable pharmaceutically acceptable salt, solvate or N-oxide thereof, may be the sole therapeutic agent in the compositions of the invention, or it may be present in the formulation in combination with one or more additional therapeutic agents. The invention thus provides, in a further aspect, a combination comprising a compound of Formula (I), or a pharmaceutically acceptable salt, solvate or N-oxide thereof together with one or more additional therapeutic agents.

The one or more additional therapeutic agent is, for example, an agent useful for the treatment of tuberculosis in a mammal. Examples of such therapeutic agents include isoniazid, ethambutol, rifampin, pirazinamide, streptomycin, capreomycin, ciprofloxacin and clofazimine.

When a compound of Formula (I), or a pharmaceutically acceptable pharmaceutically acceptable salt, solvate or N-oxide thereof is used in combination with one or more additional therapeutic agents, the dose of the compound or agent may differ from that when the compound or agent is used alone. Appropriate doses will be readily appreciated by those skilled in the art. It will be appreciated that the amount of a compound of the invention and the one or more additional therapeutic agents required for use in treatment will vary with the nature of the condition being treated and the age and the condition of the patient and will be ultimately at the discretion of the attendant physician or veterinarian.

The combinations may conveniently be presented for use in the form of a pharmaceutical formulation. In a further aspect of the present invention there is provided a pharmaceutical combination comprising a compound of Formula (I), or a pharmaceutically acceptable salt, solvate or N-oxide thereof, together with one or more additional therapeutic agents, and one or more pharmaceutically acceptable carriers, excipients or diluents. The individual components of such combinations may be administered either sequentially or simultaneously in separate or combined pharmaceutical formulations by any convenient route.

When administration is sequential, either the compound of the present invention or one or more additional therapeutic agent may be administered first. When administration is simultaneous, the combination may be administered either in the same or different pharmaceutical composition. When combined in the same formulation it will be appreciated that the compound and agents must be stable and compatible with each other and the other components of the formulation. When formulated separately they may be provided in any convenient formulation, conveniently in such manner as are known for such compounds in the art.

Abbreviations

In describing the invention, chemical elements are identified in accordance with the Periodic Table of the Elements. Abbreviations and symbols utilized herein are in accordance with the common usage of such abbreviations and symbols by those skilled in the chemical arts. The following abbreviations are used herein:
EtOAc ethyl acetate
AcOH acetic acid
Ac2O acetic anhydride
BOC N-tert-butoxycarbonyl
BOC anhydride di-tert-butyl dicarbonate
Celite® a filter aid composed of acid-washed diatomaceous silica, (a trademark of Manville Corp., Denver, Colo.)
DME dimethoxyethane
DCM dichloromethane
DIBAL-H diisobutyl aluminium hydride
DMF dimethylformamide
DMSO-d6 deuterated dimethylsulfoxide
DMSO dimethylsulfoxide
ES MS Electrospray mass spectrometry
EtOH ethanol
h hours
HPLC high performance liquid chromatography
LCMS Liquid chromatography mass spectroscopy
MeOH methanol
NaBH(OAc)₃ sodium triacetoxyborohydride
NMR Nuclear Magnetic Resonance spectroscopy
t-BuOMe methyl t-butyl ether
TFA trifluoroacetic acid
THF tetrahydrofuran
uv ultraviolet

### EXAMPLES

The following Examples illustrate the invention. These Examples are not intended to limit the scope of the invention, but rather to provide guidance to the skilled artisan to prepare and use the compounds, compositions, and methods of the invention. While particular embodiments of the invention are described, the skilled artisan will appreciate that various changes and modifications can be made. References to preparations carried out in a similar manner to, or by the general method of, other preparations, may encompass variations in routine parameters such as time, temperature, workup conditions, minor changes in reagent amounts etc.

Proton nuclear magnetic resonance CH NMR) spectra were recorded, and chemical shifts are reported in parts per million (6) downfield from the internal standard tetramethylsilane (TMS). Abbreviations for NMR data are as follows: s=singlet, d=doublet, t=triplet, q=quartet, m=multiplet, dd=doublet of doublets, dt=doublet of triplets, app=apparent, br=broad. Mass spectra were obtained using electrospray (ES) ionization techniques. All temperatures are reported in degrees centigrade.

Reactions involving metal hydrides including lithium hydride, lithium aluminium hydride, di-isobutylaluminium hydride, sodium hydride, sodium borohydride and sodium triacetoxyborohydride are carried out under argon unless otherwise specified.

Intermediates

Intermediate 1

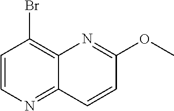

### 8-Bromo-2-(methyloxy)-1,5-naphthyridine

6-(Methyloxy)-1,5-naphthyridin-4-ol (21.5 g) (for a synthesis see WO2007016610 Preparation 2 (a)) was stirred in DMF (150 ml) at 0° C. under N₂, and phosphorous tribromide (13.5 ml) was added slowly. The mixture was allowed to warm to room temperature and stirred for 90 minutes. H₂O (375 ml) was added and the pH was adjusted to pH 7 by addition of solid Na₂CO₃. The solid was isolated by filtration with suction, dried on the sinter with suction for 2 h then dried under vacuum at 45° C. to give the desired compound (26.0 g, 90%). ¹H-NMR (δ, ppm, DMSO-d₆): 8.59 (d, 1H), 8.30 (d, 1H), 8.08 (d, 1H), 7.33 (d, 1H), 4.06 (s, 3H).

Intermediate 2

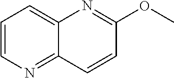

### 2-(Methyloxy)-1,5-naphthyridine

To a mixture of Intermediate 1 (25.5 g) in CH₂Cl₂ (200 ml) and EtOH (200 ml) was added NaHCO₃ (20 g) and 5% wet palladium on carbon (4 g). The resulting suspension was hydrogenated at 1.5 bar for 21 h. The mixture was filtered with suction through celite and the solids were washed with CH₂Cl₂/EtOH 1:1 (2000 ml). The combined filtrate plus washings were concentrated under reduced pressure and then treated with CH₂Cl₂/H₂O 2:1 (1600 ml). The organic phase was separated, dried over MgSO₄, filtered and evaporated under reduced pressure to give the desired compound (15.8 g, 92%). [ES MS] m/z 161 (MH⁺).

Intermediate 3

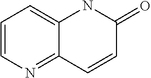

### 1,5-Naphthyridin-2(1H)-one

Intermediate 2 (15.8 g) was stirred in 6N HCl (100 ml) at 110° C. for 2 h. The mixture was cooled at 0° C. and the pH was adjusted to 6-7 with solid NaOH. The precipitated solid was isolated by filtration with suction, dried on the sinter with suction for 2 h, and dried in a vacuum at 45° C. to give the desired (14.4 g, 98%). [ES MS] m/z 147 (MH⁺).

Intermediate 4

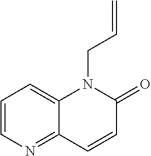

### 1-(2-Propen-1-yl)-1,5-naphthyridin-2(1H)-one

To a suspension of Intermediate 3 (5.9 g) in dry DME (180 ml) and dry DMF (45 ml) at 0° C. under argon was added in portions NaH (60% w:w dispersion in mineral oil, 3.2 g). After stirring for 45 minutes, the mixture was treated with lithium bromide (8.8 g) and the suspension was allowed to warm to room temperature. After stirring for 45 minutes, the mixture was treated with allyl bromide (7 ml) and then stirred at 65° C. for 3 h. The mixture was cooled to room temperature and concentrated under reduced pressure, then t-BuOMe (300 ml) was added and the mixture was then washed with 1N NH₄Cl (200 ml). The combined aqueous phases were extracted with t-BuOMe (2×100 ml). The organic phases were combined, washed with brine (200 ml), dried over Na₂SO₄, filtered and evaporated under reduced pressure. The residue was purified by column chromatography on silica gel using a EtOAc and hexane gradient (50-75%) to give the desired product (4.29 g, 57%). To obtain an additional amount of the desired compound, the combined aqueous phases were extracted exhaustively with CH₂Cl₂. Then, the organic extracts were combined, dried over MgSO₄, filtered and evaporated under reduced pressure. The residue was purified by column chromatography on silica gel using a EtOAc and hexane gradient (50-75%) to give the desired product (1.5 g, 20%). [ES MS] m/z 187 (MH⁺).

Intermediate 5

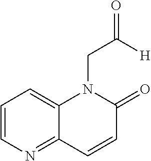

### (2-Oxo-1,5-naphthyridin-1(2H)-yl)acetaldehyde (Mixture with the Methyl Hemiacetal)

To a solution of Intermediate 4 (5.2 g) in 1,4-dioxane (100 ml) and H₂O (50 ml) was added consecutively sodium periodate (13.8 g) and osmium tetroxide (4 g of Supported OsO₄). The mixture was stirred at room temperature for 24 h. Additional amount of sodium periodate (1.4 g) and osmium tetroxide (500 mg) was added and the mixture was stirred another 72 h. The mixture was filtered and the solid washed with H₂O (250 ml) and THF (125 ml). The combined filtrate plus washings were extracted with CH₂Cl₂/MeOH (525/125; 375/125 and 375/125 ml). The organic extracts were combined, dried over Na₂SO₄, filtered and evaporated under reduced pressure to give the desired product (5.4 g, 90%). ¹H-NMR (δ, ppm, CDCl₃): 9.75 (s, 1/2H), 8.59-8.55 (m, 1H), 8.03-7.85 (m, 2H), 7.50-7.30 (m, 2H), 6.98 (d, 1H), 5.15-4.25 (m, 5H), 3.70 (s, 3H), 3.49 (s, 1/2H), 3.42-3.30 (m, 1H).

Intermediate 6

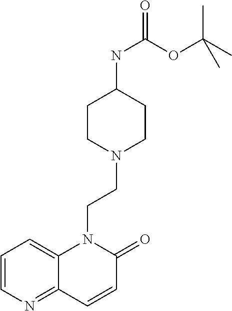

### 1,1-Dimethylethyl{1-1-[2-(2-oxo-1,5-naphthyridin-1(2H)-yl)ethyl]-4-piperidinyl}carbamate

To a mixture of Intermediate 5 (10.2 g) in CH₂Cl₂ (350 ml) and MeOH (20 ml) was added 1,1-dimethylethyl-4-piperidinylcarbamate (10.8 g, from Aldrich). After stirring for 1 h, sodium triacetoxyborohydride (34.4 g) was added. The reaction was stirred for 3 h before addition of H₂O (200 ml) and saturated NaHCO₃ (400 ml). The reaction was extracted with CH₂Cl₂/MeOH (500/75, 450/50 and 450/50 ml). The combined organic phases were dried over Na₂SO₄, evaporated and the residue was purified by chromatography on silica gel using a CH₂Cl₂ and MeOH gradient to provide the desired compound (12.7 g, 63%). [ES MS] m/z 373 (MH⁺).

Intermediate 7

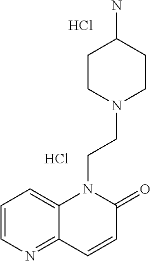

### 1-[2-(4-Amino-1-piperidinyl)ethyl]-1,5-naphthyridin-2(1H)-one dihvdrochloride

To a solution of Intermediate 6 (7.2 g) in CH₂Cl₂ (90 ml) was added HCl (4M solution in 1,4-dioxan, 35 ml). After stirring for 20 h, the obtained solid was filtered, washed with CH₂Cl₂ and dried to give 6.8 g of the title compound. ¹H-NMR (δ, ppm, D₂O): 8.52 (d, 1H), 8.22 (d, 1H), 7.99 (d, 1H), 7.82-7.78 (m,1H), 6.99 (d, 1H), 4,66-4.60 (m), 3.79-3.72 (m, 2H), 3.48-3.42 (m, 3H), 3.14-3.03 (m, 2H), 2.23-2.15 (m, 2H), 1.87-1.76 (m, 2H).

Intermediate 8

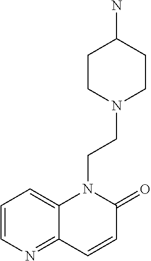

### 1-[2-(4-Amino-1-piperidinyl)ethyl]-1,5-naphthyridin-2(1H)-one

To a solution of Intermediate 7 (2 g) in H₂O (20 ml) at 0° C. was added aqueous 1N NaOH until pH 11. The reaction was then extracted with CH₂Cl₂/MeOH 95:5 (50 ml). The organic phase was dried over Na₂SO₄ and evaporated to give the desired compound (387 mg). The aqueous phase was evaporated and the residue was treated with CH₂Cl₂ (25 ml) and the mixture was stirred for 1 h. Then it was filtered and the solvent was evaporated to give an additional amount of the title compound (1.0 g). ¹H-NMR (δ, ppm, CDCl₃): 8.55 (d, 1H), 7.90 (d, 1H), 7.77 (d, 1H), 7.48-7.44 (m, 1H), 6.91 (d, 1H), 4.38 (t, 2H), 2.99-2.94 (m, 2H), 2.73-2.62 (m, 3H), 2.18 (dt, 2H), 1.87-1.76 (m, 2H); 1.43-1.30 (m, 2H), 1.25-1.13 (m, 2H).

Intermediate 9

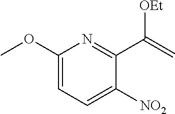

### 2-[1-(Ethyloxy)ethenyl]-6-(methyloxy)-3-nitropyridine

To a suspension of 2-chloro-6-methoxy-3-nitropyridine (600 g) and PdCl₂(PPh₃)₂ (33.5 g) in acetonitrile (4200 ml) at 65° C. under N₂ was added dropwise over 2 h (1-ethoxyvinyl)-tributyl-stannane (1182 ml). The resulting suspension was stirred at 65° C. for 4 h then left to cool to room temperature overnight. The reaction mixture was quenched with 10% KF aqueous solution (3600 ml) with vigorous stirring and stirred for 1 h. The resulting solid was removed by vacuum filtration and washed with acetonitrile (7×1000 ml). The layers were separated and the organic layer was evaporated to 3000 ml. This was filtered through Whatman, GF/B glass microfibre filter paper and the small amount of brown solid removed was washed with MeCN (1800 ml). EtOAc (3600 ml) was added and the volume reduced to 1800 ml. Cyclohexane (3600 ml) was added and the volume reduced to 3000 ml. Cyclohexane (2400 ml) and silica gel (600 g, 1 wt) were added and allowed to stir at room temperature for 1.5 h. The solid was removed by vacuum filtration and washed with EtOAc/cyclohexane, 1:8 (4200 ml). The filtrate was reduced to 1800 ml. Cyclohexane (2400 ml) was added and the volume reduced to 1800 ml. Cyclohexane (3600 ml) and EtOAc (600 ml) and silica gel (600 g, 1 wt) were added and stirred for 1.5 h. The solid was removed by vacuum filtration and washed with EtOAc/cyclohexane 1:8 (4200 ml). The solvents were evaporated to dryness. MeCN (2000 ml) was added and evaporated to give an orange coloured oil.

Intermediate 10

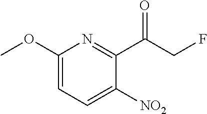

### 2-Fluoro-1-[6-(methyloxy)-3-nitro-2-pyridinyl]ethanone

To a suspension of Selectfluor (1286.4 g) in acetonitrile (2060 ml) and H₂O (820 ml) was added dropwise over 1.5 h Intermediate 9 in acetonitrile (1416 ml), maintaining the temperature <15° C. using an ice/water bath. The resulting solution was stirred at room temperature overnight. The reaction mixture was quenched with saturated aqueous NaHCO₃ (2140 ml) and stirred for 30 minutes. The volume was reduced by rotary evaporation to 3250 ml. To the resulting yellow suspension was added EtOAc (4400 ml) and H₂O (720 ml) and allowed to stir for 15 minutes. The layers were separated and the aqueous extracted with EtOAc (2×1000 ml). The organic layers were combined and washed with H₂O (1000 ml) and saturated NaCl (1000 ml). The organic layer was dried over MgSO₄ (400 g), filtered and evaporated. Acetonitrile (1000 ml) was added and evaporated to give an orange oil which slowly solidified on standing.

Intermediate 11

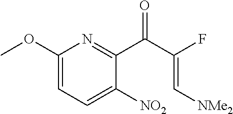

### (2Z)-3-(Dimethylamino)-2-fluoro-1-[6-(methyloxy)-3-nitro-2-pyridinyl]-2-propen-1-one

To a solution of Intermediate 10 (657.0 g) in toluene (2700 ml) under N₂ was added N,N-dimethylformamide dimethylacetal (1550 ml). The reaction mixture was heated at 50° C. for 4 h. Cyclohexane (2000 ml) was added and the reaction mixture was cooled slowly over 1 h, then to <5° C. using an ice/water bath. The precipitated solid was collected by vacuum filtration and washed with EtOAc/cyclohexane, 1:1 (3×1000 ml). The yellow solid was dried in the oven, under vacuum at 40° C. overnight.

Intermediate 12

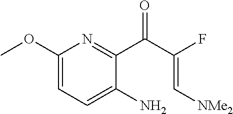

### (2Z)-1-[3-Amino-6-(methyloxy)-2-pyridinyl]-3-(dimethylamino)-2-fluoro-2-propen-1-one

To a mixture of Intermediate 11 (1146.7 g) in DMF (10500 ml) was added 5% wet palladium on carbon (274.4 g) in DMF (1000 ml). The resulting suspension was hydrogenated at 1.0 bar for 3 h, maintaining the temperature between 45-50° C. The reaction mixture was warmed to 60° C. DMF (1800 ml) was warmed to 50° C. and charged to the pressurised filter. The hot reaction mixture was nitrogen transferred through the pressurised filter, at 1.0 bar, to remove the catalyst. The vessel was rinsed out with hot DMF (2×1500 ml). The product was not isolated and used directly in the next step.

Intermediate 13

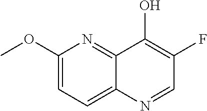

### 3-Fluoro-6-(methyloxy)-1,5-naphthyridin-4-ol

To a solution of Intermediate 12 in DMF at 0° C. was added dropwise aqueous 6N HCl (184 ml). The reaction was allowed to warm to room temperature and stirred overnight. The volume of the reaction mixture was reduced to ˜2000 ml by rotary evaporation at 50° C. and the yellow suspension was cooled to 10° C. using an ice/water bath. H₂O (4000 ml) was added slowly over 30 minutes. The reaction mixture was stirred vigorously for 1 h. The precipitated solid was collected by vacuum filtration and washed with H₂O (3000 ml) then EtOAc/cyclohexane, 1:1 (3×2000 ml). The pale brown solid was dried in the oven, under vacuum at 50° C. for 4 days.

Intermediate 14

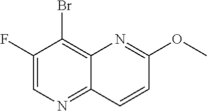

### 8-Bromo-7-fluoro-2-(methyloxy)-1,5-naphthyridine

This was prepared from Intermediate 13 (15.8 g) using a procedure analogous to that described for Intermediate 1 to give 19.6 g (93%) of the title compound. ¹H-NMR (δ, ppm, DMSO-d₆): 8.86 (s, 1H), 8.34 (d, 1H), 7.31 (d, 1H), 4.08 (s, 3H).

Intermediate 15

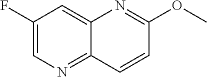

### 7-Fluoro-2-(methyloxy)-1,5-naphthyridine

This was prepared from Intermediate 14 (12.7 g) using a procedure analogous to that procedure described for Intermediate 2 to give 8.3 g (94%) of the title compound. ¹H-NMR (δ, ppm, CDCl₃): 8.67 (d, 1H), 8.18 (d, 1H), 7.81-7.76 (m, 1H), 7.07 (d, 1H), 4.08 (s, 3H).

Intermediate 16

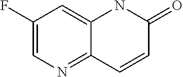

### 7-Fluoro-1,5-naphthyridin-2(1H)-one

This was prepared from Intermediate 15 (6.4 g) using a procedure analogous to that described for Intermediate 3 to give 5.7 g (95%) of the title compound. [ES MS] m/z 279 (MH⁺).

Intermediate 17

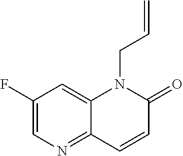

### 7-Fluoro-1-(2-propen-1-yl)-1,5-naphthyridin-2(1H)-one

This was prepared from Intermediate 16 (5.0 g) using a procedure analogous to that described for Intermediate 4 to give 5.4 g (88%) of the title compound. [ES MS] m/z 205 (MH⁺).

Intermediate 18

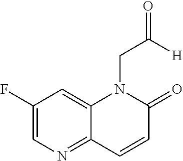

### 7-Fluoro-(2-oxo-1,5-naphthyridin-1(2H)-yl)acetaldehyde (Mixture with the methyl hemiacetal)

This was prepared from Intermediate 17 (5.4 g) using a procedure analogous to that described for Intermediate 5 to give 5.8 g of the title compound. ¹H-NMR (δ, ppm, CDCl₃): 9.77 (s, 1/2H), 8.45 (bs, 1H), 7.97 (d, 1H), 7.59 (d, 1/2H), 7.06 (d, 1H), 6.92 (d, 1H), 5.05-4.95 (m, 2H), 4.45-4.30 (m, 1H), 3.49 (s, 5/2H), 3.42 (s, 5/2H).

Intermediate 19

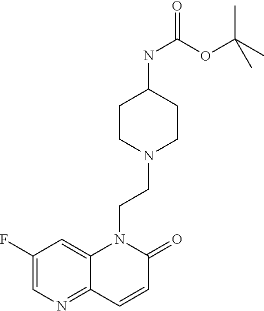

### 1,1-Dimethylethyl{1-[2-(7-fluoro-2-oxo-1,5-naphthyridin-1(2H)-yl)ethyl]-4-piperidinyl}carbamate

This was prepared from Intermediate 18 (5.8 g) using a procedure analogous to that described for Intermediate 6 to give 7.2 g (66%) of the title compound. [ES MS] m/z 391 (MH⁺).

Intermediate 20

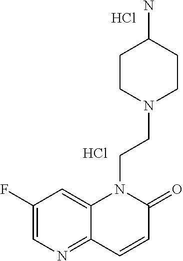

### 1-[2-(4-amino-1-piperidinyl)ethyl]-7-fluoro-1,5-naphthyridin-2(1H)-one dihydrochloride

This was prepared from Intermediate 19 (12.7 g) using a procedure analogous to that described for Intermediate 7 to give 13.5 g of the title compound. ¹H-NMR (δ, ppm, D₂O): 8.41 (s, 1H), 7.94 (d, 1H), 7.80 (d, 1H), 6.83 (d, 1H), 4.70-4.55 (m), 3.85-3.75 (m, 2H), 3.46 (t, 2H), 3.15-3.05 (m, 2H), 2.25 (bd, 2H), 1.90-1.75 (m, 2H).

Intermediate 21

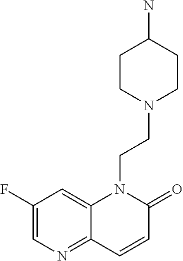

### 1-[2-(4-Amino-1-piperidinyl)ethyl]-7-fluoro-1,5-naphthyridin-2(1H)-one

To a solution of Intermediate 20 (13.5 g) in H₂O (110 ml) at 0° C. was added aqueous 2N NaOH until pH 11. The reaction was then extracted with CH₂Cl₂/MeOH 95:5 (120 ml). The organic phase was dried over Na₂SO₄ and evaporated to give the desired compound (760 mg). The aqueous phase was evaporated and the residue was treated with CH₂Cl₂/MeOH 95:5 (120 ml) and the mixture was stirred for 1 h. Then it was filtered and the solvent was evaporated to give an additional amount of the title compound (8.0 g). ¹H-NMR (δ, ppm, DMSO-d₆): 8.54 (d, 1H), 8.01 (d, 1H), 7.92 (d, 1H), 4.29 (t, 2H), 3.65-3.15 (m, 4H), 3.35 (d, 2H), 2.80-2.70 (m, 1H), 2.53-2.48 (m), 2.01 (t, 2H), 1.72 (d, 2H), 1.35-1.20 (m, 2H).

Intermediate 22

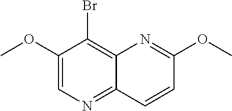

### 8-Bromo-2,7-bis(methyloxy)-1,5-naphthyridine

To a solution of Intermediate 14 (62.5 g) in dry MeOH (600 ml) under N₂ was added NaMeO (25%wt solution in MeOH, 525 ml). The mixture was stirred at 60° C. for 2 h and then, was cooled to room temperature. Brine (800 ml), H₂O (800 ml) and CH₂Cl₂ (1 L) were added. The mixture was stirred and filtered with suction. The organic phase was separated and the aqueous phase was extracted with more CH₂Cl₂ (2×500 ml). The combined organic phases were dried over Na₂SO₄ and concentrated under reduced pressure to give the desired compound (60.1 g, 92%). ¹H-NMR (δ, ppm, CDCl₃): 8.54 (s, 1H), 8.15 (d, 1H), 7.03 (d, 1H), 4.15, 4.16 (s, 6H).

Intermediate 23

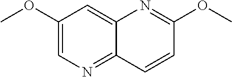

### 2,7-Bis(methyloxy)-1,5-naphthyridine

This was prepared from Intermediate 22 (15.0 g) using a procedure analogous to that described for Intermediate 2 to give 10.4 g (98%) of the title compound. ¹H-NMR (δ, ppm, CDCl₃): 8.52 (d, 1H), 8.14 (d, 1H), 7.46 (d, 1H), 6.96 (d, 1H), 4.07 (s, 3H), 3.96 (s, 3H).

Intermediate 24

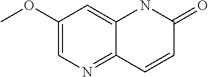

### 7-(Methyloxy)-1,5-naphthyridin-2(1H)-one

This was prepared from Intermediate 23 (39.7 g) using a procedure analogous to that described for Intermediate 3 to give 36.2 (98%) of the title compound. ¹H-NMR (δ, ppm, DMSO-d₆): 11.78 (bs, 1H), 8.19 (d, 1H), 7.84 (d, 1H), 7.12 (d, 1H), 6.52 (d, 1H), 3.86 (s, 3H).

Intermediate 25

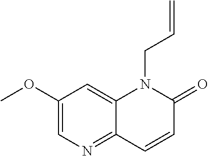

### 7-Methyloxy-1-(2-propen-1-yl)-1,5-naphthyridin-2(1H)-one

This was prepared from Intermediate 24 (15.0 g) using a procedure analogous to that described for Intermediate 4 to give 14.5 g (79%) of the title compound. ¹H-NMR (δ, ppm, CDCl₃): 8.27 (d, 1H), 7.87 (dd, 1H), 7.02 (d, 1H), 6.78 (d, 1H), 6.00-5.88 (m, 1H), 5.26 (d, 1H), 5.10 (d, 1H), 4.92-4.89 (m, 2H), 3.94 (s, 3H).

Intermediate 26

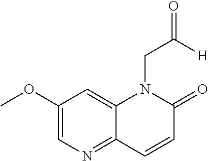

### [7-(Methyloxy)-2-oxo-1,5-naphthyridin-1(2H)-yl]acetaldehyde

To a solution of Intermediate 25 (15.4 g) in 1,4-dioxane (250 ml) and H₂O (120 ml) was added consecutively sodium periodate (35 g) and osmium tetroxide (11.3 g of Supported OsO₄). The mixture was stirred at room temperature for 24 h. Additional amount of sodium periodate (3.5 g) and osmium tetroxide (1.2 g) was added and the mixture was stirred another 72 h. The mixture was filtered and the filtrate was extracted with CH₂Cl₂/MeOH 9:1 (300, 200 and 100 ml). The organic phases were combined, dried over Na₂SO₄, filtered and evaporated under reduced pressure to give the desired product (14.9 g, 95%). ¹H-NMR (δ, ppm, CDCl₃): 9.72 (s, 1H), 8.30 (d, 1H), 7.94 (d, 1H), 6.81 (d, 1H), 6.72 (bd, 1H), 5.11 (s, 2H), 3.93 (s, 3H).

Intermediate 27

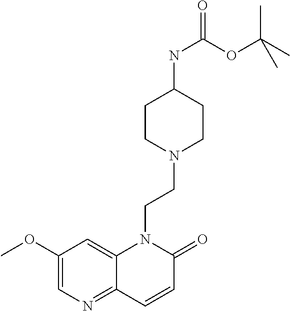

### 1,1-Dimethylethyl(1-{2-[7-(methyloxy)-2-oxo-1,5-naphthyridin-1(2H)-yl]ethyl}-4- piperidinyl)carbamate

This was prepared from Intermediate 26 (12.8 g) using a procedure analogous to that described for Intermediate 6 to give 15.3 g (65%) of the title compound. ¹H-NMR (δ, ppm, CDCl₃): 8.27 (d, 1H), 7.83 (d, 1H), 7.17 (bs, 1H), 6.73 (d, 1H), 4.44 (bs, 1H), 4.35 (t, 2H), 3.97 (s, 3H), 3.47 (bs, 1H), 2.94 (bd, 2H), 2.64 (t, 2H), 2.26 (t, 2H), 1.95 (bd, 2H), 1.70 (bs, 2H), 1.44 (s, 9H).

Intermediate 28

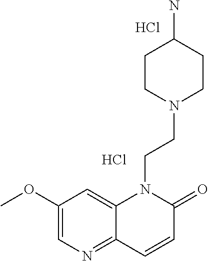

### 1-[2-(4-amino-1-piperidinyl)ethyl]-7-(methyloxy)-1,5-naphthyridin-2(1H)-one dihydrochloride

This was prepared from Intermediate 27 (15.4 g) using a procedure analogous to that described for Intermediate 7.

Intermediate 29

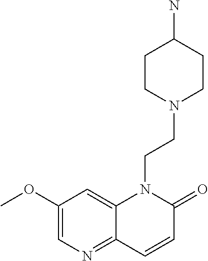

### 1-[2-(4-Amino-1-piperidinyl)ethyl]-7-methyloxy-1,5-naphthyridin-2(1H)-one as Free Base and Dihydrochloride

This was prepared from Intermediate 28 by the procedure described in Intermediate 21 to give 11.5 g (99%) of the title compound. ¹H-NMR (δ, ppm, DMSO-d₆): 8.26 (d, 1H), 7.85 (d, 1H), 7.39 (d, 1H), 6.64 (d, 1H), 4.34 (d, 2H), 3.97 (s, 3H), 3.72 (bs, 2H), 2.92 (bd, 2H), 2.74-2.64 (m, 1H), 2.54-2.48 (m), 2.03 (t, 2H), 1.72 (bd, 2H), 1.35-1.24 (m, 2H).

Intermediate 30

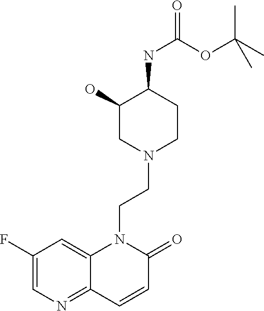

### 1,1-Dimethylethyl{(3R,4S)-1-[2-(7-fluoro-2-oxo-1,5-naphthyridin-1(2H)-yl)ethyl]-3-hydroxy-4-piperidinyl}carbamate

Intermediate 18 (200 mg) and 1,1-dimethylethyl[(3R,4S)-3-hydroxy-4-piperidinyl]carbamate (182 mg) (for a synthesis see WO2004058144, Example 5(c), cis-(3-hydroxy-piperidin-4-yl)-carbamic acid tert-butyl ester Enantiomer 1) were stirred in CHCl₃ (10 ml) plus MeOH (0.5 ml) under argon for 2 h. Sodium triacetoxyborohydride (534 mg) was added in one portion and the mixture was stirred at room temperature overnight, then quenched by addition of saturated aqueous NaHCO₃ (20 ml) and extracted with 20% v:v MeOH in CH₂Cl₂ (3×200 ml). The organic extracts were combined, dried over anhydrous MgSO₄, filtered and evaporated under reduced pressure to give the crude product, which was purified by column chromatography on silica, eluted with 0-20% (2M ammonia in MeOH) in CH₂Cl₂. Appropriate fractions were combined and evaporated under reduced pressure to give title compound (247 mg) as an off-white foam. [ES MS] m/z 407 (MH⁺).

Intermediate 31

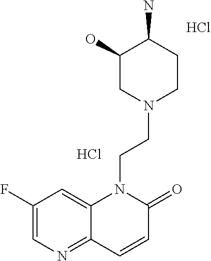

### 1-{2-[(3R,4S)-4-Amino-3-hydroxy-1-piperidinyl]ethyl}-7-fluoro-1,5-naphthyridin-2(1H)-one dihydrochloride

Intermediate 30 (240 mg) was dissolved in CH₂Cl₂ (10 ml) and the solution was treated with 4M HCl in 1,4-dioxane (2 ml). Effervescence and formation of a precipitate was observed. After 2 h, the solvents were removed under reduced pressure and the residue was dried under reduced pressure overnight, to give 220 mg of the title compound as an off-white solid. [ES MS] m/z 307 (MH⁺).

Intermediate 32

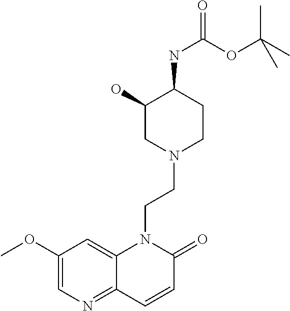

### 1,1-Dimethylethyl((3R,4S)-3-hydroxy-1-{2-[7-(methyloxy)-2-oxo-1,5-naphthyridin-1(2H)-yl]ethyl}-4-piperidinyl)carbamate

This was prepared from Intermediate 26 (200 mg) and 1,1-dimethylethyl[(3R,4S)-3-hydroxy-4-piperidinyl]carbamate (173 mg) (for a synthesis see WO2004058144, Example 5(c), cis-(3-hydroxy-piperidin-4-yl)-carbamic acid tert-butyl ester Enantiomer 1) using a procedure analogous to that described for Intermediate 30 to give 263 mg of the title compound. [ES MS] m/z 419 (MH⁺).

Intermediate 33

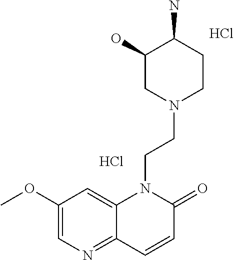

### 1-{2-[3R,4S)-4-Amino-3-hydroxy-1-piperidinyl]ethyl}-7-(methyloxy)-1,5-naohthyridin-2(1H)-one dihydrochloride

This was prepared from Intermediate 32 (258 mg) using a procedure analogous to that described for Intermediate 31 to give 223 mg of the title compound. [ES MS] m/z 319 (MH⁺).

Intermediate 34

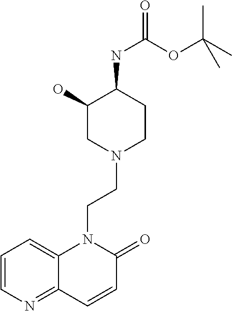

### 1,1-Dimethylethyl{(3R,4S)-3-hydroxy-1-[2-(2-oxo-1,5-naphthyridin-1(2H)-yl)ethyl]-4-piperidinyl}carbamate

This was prepared from Intermediate 5 (639 mg) and 1,1-dimethylethyl[(3R,4S)-3-hydroxy-4-piperidinyl]carbamate (250 mg) (for a synthesis see WO2004058144, Example 5(c), cis-(3-hydroxy-piperidin-4-yl)-carbamic acid tert-butyl ester Enantiomer 1) using a procedure analogous to that described for Intermediate 30 to give 250 mg of the title compound.

Intermediate 35

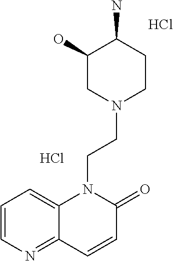

### 1-{2-[(3R,4S)-4-Amino-3-hydroxy-1-piperidinyl]ethyl}-1,5-naphthyridin-2(1H)-one dihydrochloride

This was prepared from Intermediate 34 (250 mg) using a procedure analogous to that described for Intermediate 31 to give 250 mg of the title compound.

Intermediate 35b

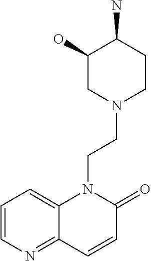

### 1-{2-[(3R,4S)-4-Amino-3-hydroxy-1-piperidinyl]ethyl}-1,5-naphthyridin-2(1H)-one

To a solution of Intermediate 35 in H₂O was added solid NaHCO₃ until pH 9. The reaction was then extracted with CH₂Cl₂/MeOH 9:1. The organic phase was dried over Na₂SO₄ and evaporated to give 28 mg of a yellow solid, which ¹H-NMR was not consistent with the desired compound. The aqueous phase was evaporated and the residue was treated with CH₂Cl₂ and the mixture was stirred at room temperature. Then it was filtered and the solvent was evaporated to provide the title compound (free base) (45 mg). ¹H-NMR (δ, ppm, CDCl₃): 8.56 (d, 1H), 7.92 (d, 1H), 7.71 (d, 1H), 7.47 (dd, 1H), 6.93 (d, 1H), 4.53-4.44 (m, 1H), 4.34-4.25 (m, 1H), 3.66 (bs, 1H), 3.09-3.06 (m, 1H), 2.85-2.81 (m, 1H), 2.72-2.65 (m, 3H), 2.34 (d, 1H), 2.26-2.17 (m, 1H), 1.64-1.57 (m, 5H).

Intermediate 36

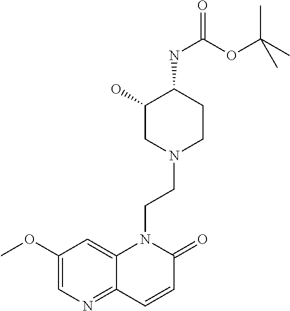

### 1,1-Dimethylethyl((3S,4R)-3-hydroxy-1-{2-[7-(methyloxy)-2-oxo-1,5-naphthyridin-1(2H)-yl]ethyl}-4-piperidinyl)carbamate

This was prepared from Intermediate 26 (200 mg) and 1,1-dimethylethyl[(3S,4R)-3-hydroxy-4-piperidinyl]carbamate (182 mg) (for a synthesis see WO2004058144, Example 5(c), cis-(3-hydroxy-piperidin-4-yl)-carbamic acid tert-butyl ester Enantiomer 2) using a procedure analogous to that described for Intermediate 30 to give 226 mg of the title compound. [ES MS] m/z 419 (MH⁺).

Intermediate 37

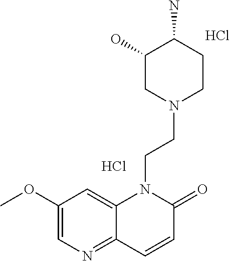

### 1-{2-[3S,4R)-4-Amino-3-hydroxy-1-piperidinyl]ethyl}-7-(methyloxy)-1,5-naphthyridin-2(1H)-one dihydrochloride

This was prepared from Intermediate 36 (223 mg) using a procedure analogous to that described for Intermediate 31 to give 209 mg of the title compound. [ES MS] m/z 319 (MH⁺).

Intermediate 38

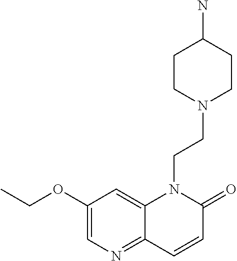

### 1-[2-(4-Amino-1-piperidinyl)ethyl]-7-(ethyloxy)-1,5-naphthyridin-2(1H)-one

Sodium hydride (124 mg) was added to a mixture of 1,4-dioxane (2 ml) and EtOH (2 ml) and it was stirred at room temperature for 5 minutes. Intermediate 21 (300 mg) was added and the mixture was microwave-heated to 160° C. for 10 minutes. H₂O was added and it was acidified with 2N HCl. The aqueous phase was washed with CH₂Cl₂/MeOH 10%, pH was adjusted to 11 with aqueous 2N NaOH. The aqueous layer was extracted with a mixture of CH₂Cl₂/MeOH 9:1, dried over Na₂SO₄ and concentrated under vacuum to give 255 mg of the title compound as a yellow oil. ¹H-NMR (δ, ppm, DMSO-d₆): 8.25 (d, 1H), 7.84 (d, 1H), 7.38 (d, 1H), 6.63 (d, 1H), 4.34-4.22 (m, 4H), 2.88-2.84 (m, 2H), 2.05-1.97 (m, 2H), 1.65-1.61 (m, 2H), 1.40 (t, 3H), 1.21-1.09 (m, 2H). [ES MS] m/z 317 (MH⁺).

Intermediate 39

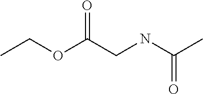

### Ethyl N-acetylglycinate

To a solution of N-acetylglycine (3 g) in EtOH (40 ml) under Ar was added p-toluenesulfonic acid (441 mg). The mixture was heated at 90° C. overnight. The cooled mixture was concentrated under vacuum and the residue was partitioned between CH₂Cl₂ and saturated NaHCO₃. The organic phase was concentrated under vacuum to give 2.5 g (69%) of the title compound. ¹H-NMR (δ, ppm, CDCl₃): 6.00 (bs, 1H), 4.23 (q, 2H), 4.03 (d, 2H), 2.05 (s, 3H), 1.30 (t, 3H).

Intermediate 40

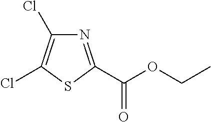

### Ethyl 4,5-dichloro-1,3-thiazole-2-carboxylate

A solution of Intermediate 39 (1.2 g) and thionyl chloride (3.2 ml) in anhydrous benzene (10 ml) was heated under reflux for 1 h. The cooled mixture was concentrated under vacuum. The residue obtained was purified by column chromatography on silica gel using hexane/EtOAc 9:1 as eluent to give 500 mg (26%) of the title compound. ¹H-NMR (δ, ppm, CDCl₃): 4.48 (q, 2H), 1.43 (t, 3H). [ES MS] m/z 226 (MH+).

Intermediate 41

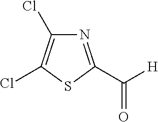

### 4,5-Dichloro-1,3-thiazole-2-carbaldehyde

To a solution of Intermediate 40 (500 mg) in CH₂Cl₂ (7 ml) at −78° C. under N₂ was added dropwise di-isobutyl aluminium hydride (1.5 M solution in toluene, 1.6 ml). The solution was stirred at −78° C. for 45 minutes. Then, more DIBAL-H (1.5 M solution in toluene, 1.0 ml) was added. The reaction was stirred at −78° C. for 30 minutes. To this solution was added slowly a mixture of MeOH/acetic acid 2:1 (6 ml) followed by H₂O/CH₂Cl₂. The combined organic phases were washed with brine, and concentrated in vacuo to give 76.3 mg of the title compound.

¹H-NMR (δ, ppm, CDCl₃): 9.73 (s, 1H).

Intermediate 42

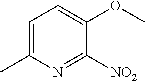

### 6-Methyl-3-(methyloxy)-2-nitropyridine

To a solution of 6-methyl-2-nitro-3-pyridinol (2.5 g) in DMF (40 ml) under N₂ was added a solution of NaH (60% w:w dispersion in mineral oil, 714 mg) in DMF (10 ml). The reaction was heated under reflux for 1.5 h and then cooled to room temperature. To this solution iodomethane (1.05 ml) was added. The mixture was stirred at room temperature overnight. Then, more iodomethane (0.28 ml) was added. After stirring for 1.5 h, isopropanol (10 ml) was added and the mixture was stirred for 30 minutes. The mixture was concentrated under vacuum and the residue was partitioned between H₂O and EtOAc. The combined organic phases were dried over Na₂SO₄, filtered, and concentrated under vacuum. The residue obtained was purified by column chromatography on silica gel using hexane/EtOAc as eluent to give the title compound together with starting material. The obtained solid was partitioned between CH₂Cl₂/Na₂CO₃ 9:1. The combined organic phases were dried over Na₂SO₄, filtered, and concentrated under vacuum to give 2.3 g of the title compound as a yellow solid. ¹H-NMR (δ, ppm, CDCl₃): 7.39 (q, 2H), 3.94 (s, 3H), 2.53 (s, 3H).

Intermediate 43

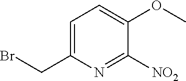

### 6-(Bromomethyl)-3-(methyloxy)-2-nitropyridine

To a solution of Intermediate 42 (500 mg) in CCl₄ (10 ml) was added N-bromosuccinimide (525 mg) and benzoyl peroxide (35.8 mg). The mixture was heated under reflux overnight. Then, more benzoyl peroxide (35.8 mg) was added. After refluxing for 24 h, the solvent was evaporated under vacuum and the residue obtained was purified by column chromatography on silica gel using hexane/EtOAc as eluent to give 109 mg of the title compound. ¹H-NMR (δ, ppm, CDCl₃): 7.70 (d, 1H), 7.51 (d, 1H), 4.52 (s, 2H), 3.98 (s, 3H).

Intermediate 44

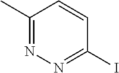

### 3-lodo-6-methylpyridazine

To a solution of 3-chloro-6-methylpyridazine (500 mg) in Hl (3 ml) was added Nal (782 mg). The reaction was heated at 40° C. for 4 hours and at 70° C. overnight. The yellow precipitate was filtered. The mother liquors were basified using solid NaOH, extracted with CH₂Cl₂, dried over MgSO₄, filtered, concentrated, and purified by chromatography on silica gel using EtOAc/hexane as eluent to give 171.7 mg (20%) of the title compound as a white solid almost pure by HPLC. The yellow precipitate was basified using the same aqueous phase, extracted with CH₂Cl₂, dried over MgSO₄, filtered and concentrated, to give 579.1 mg (68%) of the pure title compound as a white solid. ¹H-NMR (δ, ppm, CDCl₃): 7.74 (1H, d), 7.02 (1H, d), 2.66 (3H, s). [ES MS] m/z 221 (MH⁺).

Intermediate 45

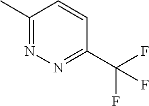

### 3-Methyl-6-(trifluoromethyl)pyridazine

A mixture of Cul (263 mg) and KF (80 mg) was heated under vacuum for 30 minutes until a greenish colour was observed. The system was filled with N₂ and a solution of Intermediate 44 (300 mg) in DMF (1.25 ml) and N-methylpirrolidinone (1.25 ml) followed by trimethyl(trifluoromethyl)silane (185 μl) were subsequently added. A dark brown colour was observed. After stirring at room temperature for 5 days, the reaction was not complete. Cul (263 mg), KF (80 mg), and CF₃TMS (185 μl) were added and stirring was continued overnight. Additional Cul (135 mg), KF (40 mg), and CF₃TMS (90 μl) were added and after one more day at room temperature, almost no starting material was observed. Aqueous ammonia and t-BuOMe were added and the phases were separated. The aqueous one was washed with t-BuOMe. The organic extracts were combined and subsequently washed with aqueous ammonia, 1M HCl, saturated NaHCO₃, and brine, dried over Na₂SO₄, filtered and concentrated to give 70 mg (35%) of the title compound. ¹H-NMR (δ, ppm, CDCl₃): 7.72 (1H, d), 7.53 (1H, d), 2.85 (3H, s). [ES MS] m/z 163 (MH⁺).

Intermediate 46

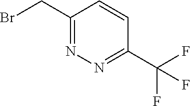

### 3-(Bromomethyl)-6-(trifluoromethyl)pyridazine

A mixture of Intermediate 45 (70 mg), N-bromosuccinimide (84 mg), azobisisobutyronitrile (14.1 mg), and CCl₄ (3 ml) was heated under reflux for 2 days. The solvent was evaporated to dryness under vacuum. The residue obtained was purified by chromatography column on silica gel (AIT, Flashsmart BP-SUP, 20-40 μm) using a mixture of EtOAc/hexane as eluent to give 14 mg (14%) of the title compound along with 15.3 mg (11%) of the dibrominated derivative. ¹H-NMR (δ, ppm, CDCl₃): 7.90 (1H, d), 7.86 (1H, d), 4.83 (2H, s). [ES MS] m/z 241 and 243 (MH⁺).

Intermediate 47

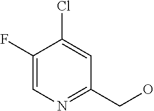

### (4-Chloro-5-fluoro-2-pyridinyl)methanol

4-Chloro-5-fluoro-2-methylpyridine 1-oxide (840 mg) (for a synthesis see J. Med. Chem., 1989, 32, 1970-1977) was refluxed in Ac₂O (6 ml) for 1 h. After adding H₂O, the pH was adjusted to 9 with aqueous 2N NaOH. The aqueous phase was extracted with CH₂Cl₂, dried over Na₂SO₄, filtered and concentrated under vacuum to give 700 mg of a mixture of the (4-chloro-5-fluoro-2-pyridinyl)methyl acetate (majority) and (4-chloro-5-fluoro-2-pyridinyl)methanol. The mixture was dissolved in aqueous 35% HCl (4 ml) and H₂O (4 ml) and refluxed for 30 minutes. After adding H₂O, the pH was adjusted to 9-14 with aqueous 2N NaOH. The aqueous phase was extracted with CH₂Cl₂, dried over Na₂SO₄, filtered and concentrated under vacuum to give 475 mg of the title compound as a colourless oil. ¹H-NMR (δ, ppm, CDCl₃): 8.43 (s, 1H), 7.41 (d, 1H), 4.74 (d, 2H).

Intermediate 48

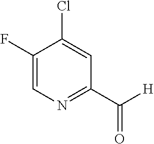

### 4-Chloro-5-fluoro-2-pyridinecarbaldehyde

To a solution of Intermediate 47 (200 mg) in CH₂Cl₂ (5 ml) was added manganese oxide (861 mg). The suspension was stirred overnight and filtered eluting with CH₂Cl₂. The filtrate was concentrated in vacuo to afford 130 mg (66%) of the title compound as a yellow oil. ¹H-NMR (δ, ppm, CDCl₃): 10.00 (s, 1H), 8.64 (s, 1H), 8.06 (d, 1H).

Intermediate b 49

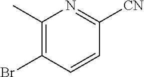

### 5-Bromo-6-methyl-2-pyridinecarbonitrile

A mixture of 3,6-dibromo-2-methylpyridine (200 mg), zinc cyanide (140 mg) and tetrakis(triphenylphosphine)palladium (0) (92.4 mg) in DMF (4 ml) was heated at 100° C. for 10 h. Then, an excess of tetrakis(triphenylphosphine)palladium (0) (93 mg) was added. The mixture was heated at 140° C. for 20 h. After cooling, the reaction was filtered through a pad of celite, diluted with EtOAc and washed with H₂O (×3). The combined aqueous layers were then washed with EtOAc (×3). The combined organic layers were washed with brine, dried over MgSO₄, filtered and concentrated under vacuum to give 314 mg of a brown sticky solid. This was purified on Biotage using hexane/EtOAc 8:2 as eluent to give 89 mg of the title compound as a white solid. [ES MS] m/z 197 (MH+).

Intermediate 50

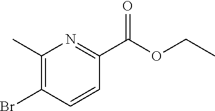

### Ethyl 5-bromo-6-methyl-2-pyridinecarboxylate

A mixture of Intermediate 49 (89 mg) and borane-tetrahydrofuran complex (0.23 ml) in EtOH (2 ml) was refluxed under argon overnight. More borane-tetrahydrofuran complex in EtOH was added until starting material was not detected by LCMS. The solvent was removed in vacuo, the residue was partitioned between CH₂Cl₂ and H₂O and passed through a phase separator with a Na₂SO₄ cartridge attached. The solvent was removed in vacuo to give 87 mg of the title compound as a yellow solid. [ES MS] m/z 266 (M+Na).

Intermediate 51

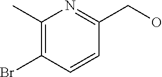

### (5-Bromo-6-methyl-2-pyridinyl)methanol

To a suspension of Intermediate 50 (200 mg) in CH₂Cl₂ (2.5 ml) at 0° C. was added di-isobutyl aluminium hydride (1.5M solution in toluene, 1.1 ml). The solution was stirred at 0° C. for 8 h. Then, more DIBAL-H (1.5 M solution in toluene, 1.1 ml) was added. The reaction was stirred at room temperature overnight. To this solution was added saturated, aqueous NaK-tartrate solution followed by CH₂Cl₂. This emulsion was vigorously stirred. The phases were separated and the aqueous phase was extracted with CH₂Cl₂. The combined organic phases were washed with brine, dried over MgSO₄, filtered and concentrated in vacuo to afford the desired product (155 mg, 96%). ¹H-NMR (δ, ppm, CDCl₃): 7.78 (d, 1H), 6.96 (d, 1H), 4.68 (d, 2H), 3.55 (t, 1H), 2.67 (s, 3H). [ES MS] m/z 202 (MH+).

Intermediate 52

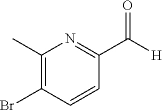

### 5-Bromo-6-methyl-2-pyridinecarbaldehyde

This was prepared from Intermediate 51 (155 mg) using a procedure analogous to that described for Intermediate 48 to give 81 mg (53%) of the title compound. ¹H-NMR (δ, ppm, CDCl₃): 10.01 (s, 1H), 8.00 (d, 1H), 7.66 (d, 1H), 2.78 (s, 3H).

Intermediate 53

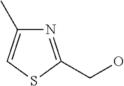

### (4-Methyl-1,3-thiazol-2-yl)methanol

To a solution of 4-methyl-1,3-thiazole-2-carbaldehyde (300 mg) in THF (3 ml) and EtOH (3 ml) at 0° C. was added sodiumborohydride (267 mg). The mixture was stirred at room temperature overnight. The solvent was evaporated under vacuum and the residue was suspended in CH₂Cl₂. The suspension was stirred for 2 h and the obtained salts were filtered off. The filtrate was concentrated under vacuum to give 252 mg of the title compound as a yellow solid. ¹H-NMR (δ, ppm, CDCl₃): 7.43 (d, 1H), 7.37(d, 1H), 3.94 (s, 3H), 2.53(s, 3H).

Intermediate 54

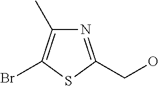

### (5-Bromo-4-methyl-1,3-thiazol-2-yl)methanol

To a solution of Intermediate 53 (252 mg) in DMF was added N-bromosuccinimide (385 mg). The mixture was heated under reflux overnight. The solvent was evaporated under vacuum and the residue obtained was purified by column chromatography on silica gel using hexane/EtOAc as eluent to give 95 mg of the title compound as a brown solid. ¹H-NMR (δ, ppm, CDCl₃): 4.86 (s, 2H), 2.38 (s, 3H).

Intermediate 55

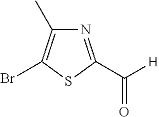

### 5-Bromo-4-methyl-1,3-thiazole-2-carbaldehyde

To a solution of Intermediate 54 (95 mg) in CH₂Cl₂ (10 ml) was added manganese oxide (118 mg). The mixture was heated under reflux overnight. Then, more Mn0₂ (79 mg) was added. After refluxing overnight, the cooled solution was filtered through celite and the corresponding filtrate was concentrated under vacuum. The residue obtained was purified by column chromatography on silica gel using hexane/EtOAc as eluent to give 50 mg of the title compound as a yellow oil. ¹H-NMR (δ, ppm, CDCl₃): 9.82 (s, 1H), 2.52 (s, 3H).

Intermediate 56

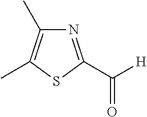

### 4,5-Dimethyl-1,3-thiazole-2-carbaldehvde

To a solution of butyllithium (1.6M solution in hexanes, 2.2 ml) in THF (10 ml) at −78° C. was added dropwise 4,5-dimethyl-1,3-thiazole (360 μl). After stirring at -78° C. for 1 h, DMF (286 μl) was added and the reaction was allowed to warm to room temperature. The solvent was evaporated under vacuum. H₂O and saturated NaCl were added and aqueous phase was washed with CH₂Cl₂ several times. The combined organic layers were dried over NaSO₄, filtered, and concentrated under vacuum to give 310 mg of the title compound as a yellow oil.¹H-NMR (δ, ppm, CDCl₃): 9.84 (s, 1H), 2.47(s, 3H), 2.44 (s, 3H).

Interemdiate 57

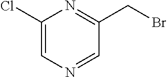

### 2-(Bromomethyl)-6-chloropyrazine

This was prepared from 2-chloro-6-methylpyrazine (200 mg) by the procedure described in Intermediate 46. The residue obtained was purified by column chromatography on silica gel using CH₂Cl₂/hexane as eluent to give 55 mg (17%) of the title compound along with 15.3 mg (11%) of the dibrominated derivative. ¹H-NMR (δ, ppm, CDCl₃): 8.63 (s, 1H) 8.54 (s, 1H), 4.52 (s, 2H).

Intermediate 58

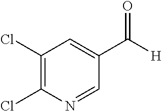

### 5,6-Dichloro-3-pyridinecarbaldehyde

To a solution of (5,6-dichloro-3-pyridinyl)methanol (200 mg) in CH₂Cl₂, molecular sieves (3A) (55 mg) and 4-methy-morpholine-N-oxide (198 mg) was added and stirred at room temperature for 10 minutes. Then tetra-n-propylammonium perruthenate(VII) (38 mg) was added and stirred overnight. The reaction mixture was filtered and the solvent was evaporated to dryness under vacuum. The residue obtained was purified by chromatography column on silica gel using a mixture of EtOAc/hexane as eluent to give 44 mg (22%) of the title compound. ¹H-NMR (δ, ppm, CDCl₃): 10.09 (s, 1H), 8.77 (s, 1H), 8.25 (s, 1H).

Interemediate 59

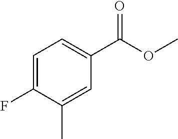

### Methyl 4-fluoro-3-methylbenzoate

To a suspension of 4-fluoro-3-methylbenzoic acid (300 mg) in MeOH (4.5 ml) was added dropwise thionyl chloride (142 μl). The solution was stirred at room temperature overnight and concentrated in vacuo to give 297 mg of the title compound. ¹H-NMR (δ, ppm, CDCl₃): 7.92-7.84 (m, 2H), 7.05 (t, 1H), 3.91 (s, 3H), 2.32 (d, 3H).

Intermediate 60

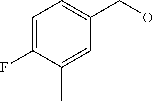

### (4-Fluoro-3-methylphenyl)methanol

This was prepared from 59 (297 mg) using a procedure analogous to that described for Intermediate 51 to give 240 mg (97%) of the title compound. ¹H-NMR (δ, ppm, CDCl₃): 7.24-7.16 (m, 2H), 6.98 (t, 1H), 4.63 (d, 2H), 2.28 (s, 3H).

Intermediate 61

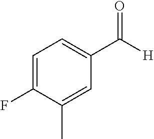

### 4-Fluoro-3-methylbenzaldehyde

This was prepared from Intermediate 60 (240 mg) using a procedure analogous to that described for Intermediate 48 to give 150 mg (61%) of the title compound. ¹H-NMR (δ, ppm, CDCl₃): 9.94 (s, 1H), 7.77-7.70 (m, 2H), 7.16 (t, 1H), 2.37 (d, 3H).

Intermediate 62

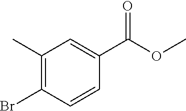

### 4-Bromo-3-methylbenzoic acid

This was prepared from 4-bromo-3-methylbenzoic acid (300 mg) using a procedure analogous to that described for Intermediate 59 to give 315 mg (98%) of the title compound. ¹H-NMR (δ, ppm, CDCl₃): 7.91 (d, 1H), 7.71 (dd, 1H), 7.61 (d, 1H), 3.92 (s, 3H), 2.45 (s, 3H).

Intermediate 63

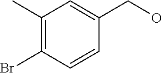

### (4-Bromo-3-methylphenyl)methanol

This was prepared from Intermediate 62 (300 mg) using a procedure analogous to that described for Intermediate 51 to give 212 mg (81%) of the title compound. ¹H-NMR (δ, ppm, CDCl₃): 7.52 (d, 1H), 7.25 (d, 1H), 7.05 (dd, 1H), 4.64 (d, 2H), 2.41 (s, 3H).

Intermediate 64

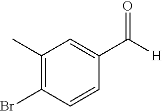

### 4-Bromo-3-methylbenzaldehyde

This was prepared from Intermediate 63 (205 mg) using a procedure analogous to that described for Intermediate 58 to give 62 mg (31%) of the title compound. ¹H-NMR (δ, ppm, CDCl₃): 9.96 (s, 1H), 7.73 (d, 1H), 7.56 (dd, 1H), 2.49 (s, 3H).

Intermediate 65

### 2-(trifluoromethyl)-4-pyrimidinecarbaldehyde (Used to Prepare Example 113)

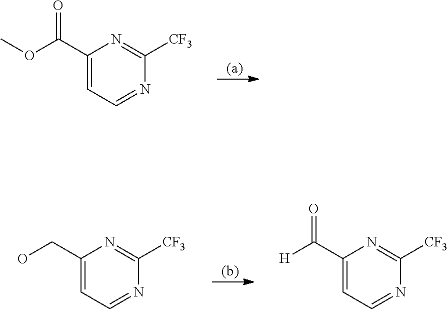

### (a) [2-(Trifluoromethyl)-4-pyrimidinyl]methanol

To a solution of methyl 2-trifluoromethyl-4-pyrimidine carboxylate (200 mg, 0.97 mmol) in DCM (5 ml), DIBAL-H (1.5 M solution in toluene, 1.62 ml, 2.43 mmol) was added dropwise under nitrogen at 0° C. After stirring at 0° C. for 6 h, TLC showed no starting material. The reaction was quenched by addition of MeOH. Then solvent was eliminated under vacuum and the crude of reaction was dissolved in DCM and the organic phase was washed with 1N HCl, dried over Na₂SO₄, filtered and concentrated under vacuum to give [2-(trifluoromethyl)-4-pyrimidinyl]methanol (103 mg, 0.578 mmol, 60%) as a brown oil pure enough to be used in the next step.

¹H-NMR (δ, ppm, CDCl₃): 8.90 (1H, d), 7.63 (1H, d), 4.90 (2H, s).

### (b) Title Compound: 2-(trifluoromethyl)-4-pyrimidinecarbaldehyde

To a solution of [2-(trifluoromethyl)-4-pyrimidinyl]methanol (62 mg, 0.35 mmol) in CH₂Cl₂ (5 ml), dess-martin periodinane (177 mg, 0.42 mmol) was added and stirred at room temperature for 6 h. Additional amount of dess-martin periodinane (885 mg) was added and the mixture was stirred another 24 h. The reaction was quenched by addition of saturated solution of NaHCO3. The organic layer was dried over Na₂SO₄, filtered and concentrated under vacuum to give 2-(trifluoromethyl)-4-pyrimidinecarbaldehyde (113 mg, 0.642 mmol, 95%) as a yellow oil pure enough to be used in the next step.

¹H-NMR (δ, ppm, CDCl₃): 10.13 (1H, s), 8.29 (1H, d), 8.02 (1H, d).

Intermediate 66.

### 4,6-Dimethyl-2-pyridinecarbaldehyde (Used to Prepare Example 114)

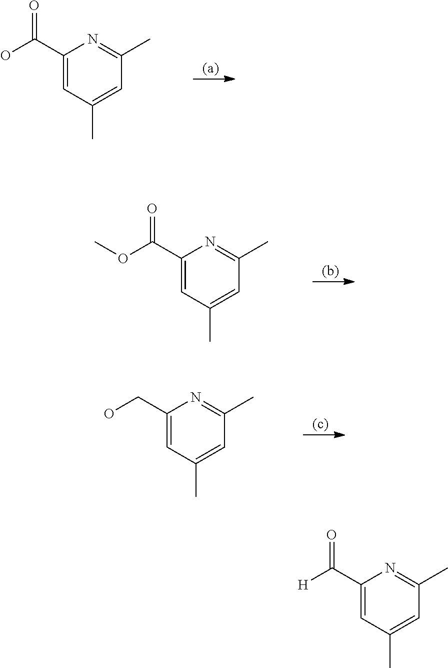

### (a) Methyl 4,6-dimethyl-2-pyridinecarboxylate

Thionyl chloride (0.193 ml, 2.65 mmol) was carefully added to a solution of 4,6-dimethyl-2-pyridinecarboxylic acid (400 mg, 2.65 mmol) in methanol (6 ml) at rt and the mixture was stirred at that temperature. The reaction was monitored by LCMS. More thionyl chloride (0.193 ml, 2.65 mmol) was added next day. After 8 days, LCMS showed almost full conversion. The solvent was removed under vacuum yielding methyl 4,6-dimethyl-2-pyridinecarboxylate (575 mg, 2.62 mmol, 99% yield) pure enough to be used in the next step.

¹H-NMR (δ, ppm, CDCl₃): 8.10 (s, 1H), 7.64 (s, 1H), 4.13 (s, 3H), 3.20 (s, 3H).

### (b) (4,6-Dimethyl-2-pyridinyl)metanol

Lithium aluminum hydride (3.94 ml, 3.94 mmol) was slowly added to a solution of methyl 4,6-dimethyl-2-pyridinecarboxylate (575 mg, 2.62 mmol) hydrochloride in tetrahydrofuran (8 ml) and dichloromethane (4 ml) at 0° C. under nitrogen and the mixture was warmed up to rt overnight. After 18 hr LCMS showed almost full conversion. The reaction was cooled to 0° C. and ethyl acetate and saturated Na—K tartrate solution were added. After extraction, the organic layer was dried (MgSO₄), filtered and concentrated. (4,6-dimethyl-2-pyridinyl)methanol (306.2 mg, 2.232 mmol, 85% yield) was obtained as a brown solid pure enough to be used in the next step.

¹H-NMR (δ, ppm, CDCl₃): 6.86 (s, 1H), 6.84 (s, 1H), 4.67 (s, 2H), 2.50 (s, 3H), 2.29 (s, 3H).

### (c) Title Compound: 4,6-dimethyl-2-pyridinecarbaldehyde

Manganese dioxide (776 mg, 8.92 mmol) was added to a solution of (4,6-dimethyl-2-pyridinyl)methanol (306 mg, 2.231 mmol) in Dichloromethane (DCM) (10 ml) at rt and the mixture was stirred at that temperature. The progress was monitored by TLC (10% MeOH in DCM). Two more additions of Manganese dioxide (776 mg, 8.92 mmol) and an overall time of 48 hr was necessary to get almost full conversion. The solids were filtered off and the solvent evaporated. 4,6-dimethyl-2-pyridinecarbaldehyde (192 mg, 1.421 mmol, 63.7% yield) was obtained pure enough to be used in the next step. ¹H-NMR (δ, ppm, CDCl₃): 10.03 (s, 1H), 7.60 (s, 1H), 7.18 (s, 1H), 2.60 (s, 3H), 2.37 (s, 3H).

Intermediate 67.

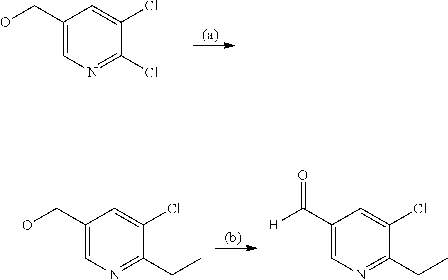

### 5-chloro-6-ethyl-3-pyridinecarbaldehyde (Used to Prepare Example 127)

### (a) (5-Chloro-6-ethyl-3-pyridinyl)metanol

To a mixture of 5,6-dichloro-3-pyridnemethanol (500 mg, 2.81 mmol), potassium carbonate (1165 mg, 8.43 mmol), [1,1′-bis(diphenylphosphino)ferrocene]dichloro-palladium(11) (206 mg, 0.281 mmol), and tetrahydrofuran (16.8 ml) was added diethylzinc (5.67 ml, 5.67 mmol). The reaction was heated under reflux for 16 h, then cooled to rt, quenched by addition of aq. HCl until pH=7 and extracted with EtOAc. The combined organic layers were washed with brine, dried over MgSO₄ filtered, and evaporated. The crude reaction was purified by flash cromatography using mixtures of EtOAc/Hexane as eluants to afford the desired product (5-chloro-6-ethyl-3-pyridinyl)methanol (103 mg, 0.600 mmol, 21.37% yield).

¹H-NMR (δ, ppm, CDCl₃): 8.34 (s, 1H), 7.69 (s, 1H), 4.68 (s, 2H), 2.94 (q, 2H), 1.27 (t, 3H). [ES MS] m/z 172 (MH+).

### (b) Title Compound: 5-chloro-6-ethyl-3-pyridinecarbaldehyde

(5-chloro-6-ethyl-3-pyridinyl)methanol (103 mg, 0.600 mmol) was dissolved in dichloromethane (4 ml) and manganese(IV) oxide (620 mg, 7.13 mmol) was added. The reaction mixture was stirred overnight. Manganese (IV) oxide (261 mg, 3.00 mmol) was added. After 3 hours the reaction went to completion. The reaction mixture was filtered and evaporated to afford 5-chloro-6-ethyl-3-pyridinecarbaldehyde (52 mg, 0.307 mmol, 51.1% yield) pure enough to be used in the next step.

¹H-NMR (δ, ppm, CDCl₃): 10.07 (s, 1H), 8.89 (s, 1H), 8.07 (s, 1H), 3.08 (q, 2H), 1.31 (t, 3H).

Intermediate 68

### 5,6-Dimethyl-3-pyridinecarbaldehyde (used to Prepare Example 125)

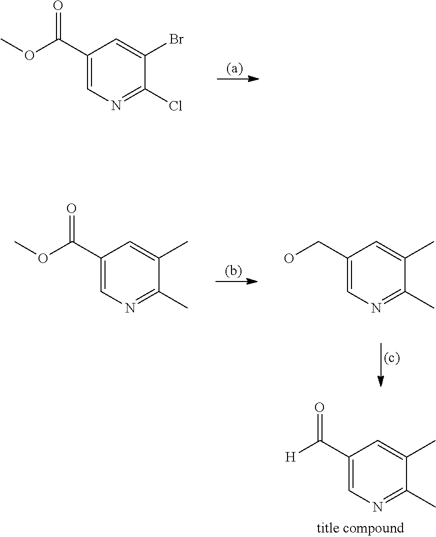

### (a) Methyl 5,6-dimethyl-3-pyridinecarboxylate

To a mixture of methyl 5-bromo-6-chloro-3-pyridinecarboxylate (595 mg, 2.375 mmol), tetrakis(triphenylphosphine)palladium (0) (275 mg, 0.238 mmol) and potassium carbonate (492 mg, 3.56 mmol) in dioxane (6.5 ml), trimethylboroxin (1.328 ml, 9.50 mmol) was added under argon. The reaction mixture was heated at 110° C. for 16 hours. The reaction mixture was allowed to cool down, filtered through a pad of silica, and concentrated to give 857 mg of the crude material. Purification by flash chromatography using Flashmaster II, a 20 g silica gel cartridge, and mixtures of hexane and EtOAc as eluent afforded 276 mg of desired compound.

### (b) (5,6-Dimethyl-3-pyridinyl)methanol

To a solution of methyl 5,6-dimethyl-3-pyridinecarboxylate (276 mg, 1.671 mmol) in DCM (6 ml), DIBAL-H (1.5 M solution in toluene, 3.34 ml, 5.01 mmol) was added dropwise at −78° C. under nitrogen. The reaction mixture was allowed to warm to rt and stirred overnight. To this solution was added saturated, aqueous NaK-tartrate solution followed by CH₂Cl₂.

The organic phase was separated, dried and concentrated to afford 175 mg of the crude material. Purification by flash chromatography using Flashmaster II, a 5 g silica gel cartridge, and mixtures of DCM/MeOH as eluent afforded 105 mg of desired compound.

### (c) Title compound: 5,6-dimethyl-3-pyridinecarbaldehyde

To a solution of (5,6-dimethyl-3-pyridinyl)methanol (105 mg, 0.765 mmol) in DCM (4 ml), MnO₂ (532 mg, 6.12 mmol) was added. After stirring overnight, an excess of MnO₂ (333 mg, 3.83 mmol) was added and then the mixture was stirred for 3 hours more. The reaction mixture was filtrathed and evaporated to afford 51 mg of title compound.

¹H-NMRδ, ppm, CDCl₃): 10.05 (s, 1H), 8.75 (s, 1H), 7.87 (s, 1H), 2.60 (s, 3H), 2.30 (s, 3H).

Intermediate 69

### 5-Bromo-6-methyl-3-pyridinecarbaldehyde (used to Prepare Examples 126)

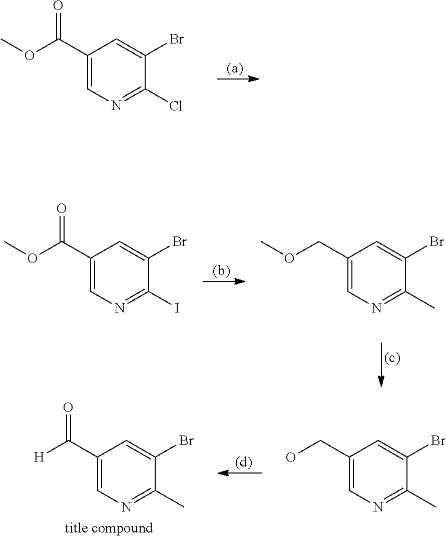

### (a) Methyl 5-bromo-6-iodo-3-pyridinecarboxylate

To a mixture of methyl 5-bromo-6-chloro-3-pyridinecarboxylate (1 g, 3.99 mmol) in propionitrile (30 ml) was added iodotrimethylsilane (0.689 ml, 3.99 mmol) and then sodium iodide (1.795 g, 11.98 mmol). The mixture was stirred at room temperature for 30 min. Solvent was evaporated and the obtained solid was dissolved in H₂O. pH was adjusted to basic with 2M NaOH and DCM was added. The organic phase was extracted, dried over Na₂SO₄, filtered and concentrated to give 1.2 g (79%) of desired compound as a yellow solid.

¹H-NMR(δ, ppm, CDCl₃): 8.84 (s, 1H), 8.35 (s, 1H), 3.96 (s, 3H).

### (b) Methyl 5-bromo-6-methyl-3-pyridinecarboxylate

### Method A

To a mixture of methyl 5-bromo-6-iodo-3-pyridinecarboxylate (232 mg, 0.679 mmol), tetrakis(triphenylphosphine)palladium (0) (39.2 mg, 0.034 mmol), and potassium carbonate (281 mg, 2.036 mmol) in dioxane (5 ml), trimethylboroxin (0.095 ml, 0.679 mmol) was added under nitrogen. The reaction mixture was heated at 110° C. and more tetrakis(triphenylphosphine)palladium (0) was added in several portions until starting material was not detected by HPLC. The reaction mixture was diluted with water and extracted three times with EtOAc. The combined organic phases were washed with brine, dried over anhydrous Na₂SO₄ and concentrated under reduced pressure. The crude of reaction was purified by flash chromatography using Flashmaster II, a 5 g silica gel cartridge, and mixtures of hexane and EtOAc to afford the title compound (75 mg, 48%) as a yellow solid.

¹H-NMR(δ, ppm, CDCl₃): 9.01 (s, 1H), 8.41 (s, 1H), 3.95 (s, 3H), 2.74 (s, 3H). MS (ES+) m/z 230 (MH+).

### Method B

To a mixture of methyl 5-bromo-6-chloro-3-pyridinecarboxylate (400 mg, 1.597 mmol), tetrakis(triphenylphosphine)palladium (0) (185 mg, 0.160 mmol), and potassium carbonate (331 mg, 2.395 mmol) in dioxane (5 ml), trimethylboroxin (0.446 ml, 3.19 mmol) was added under argon. The reaction mixture was heated at 110° C. for 5 hours. The reaction mixture was allowed to cool down, filtered through a pad of silica, and concentrated to give 176 mg of the crude material. Purification by flash chromatography using Flashmaster II, a 5 g espherical silica gel cartridge, and mixtures of hexane and DCM as eluent afforded 76 mg of desired compound and 84 mg of methyl 6-chloro-5-methyl-3-pyridinecarboxylate as byproduct.

MS (ES+) m/z 230 (MH+).

### (c) (5-Bromo-6-methyl-3-pyridinyl)methanol

To a solution of methyl 5-bromo-6-methyl-3-pyridinecarboxylate (76 mg, 0.330 mmol) in DCM (1.5 ml), DIBAL-H (1.5 M solution in toluene, 0.661 ml, 0.991 mmol) was added dropwise at −78° C. under nitrogen. The reaction mixture was allowed to warm to rt and stirred overnight. To this solution was added saturated, aqueous NaK-tartrate solution followed by CH₂Cl₂. The organic phase was separated, dried and concentrated to afford 56 mg of 5-bromo-6-methyl-3-pyridinyl)methanol (56 mg, 84%) pure enough to use in the next step.

¹H-NMR(δ, ppm, CDCl₃): 8.45 (s, 1H), 7.97 (s, 1H), 4.73 (s, 2H), 2.70 (s, 3H).

### (d) Title compound: 5-bromo-6-methyl-3-pyridinecarbaldehyde

To a solution of (5-bromo-6-methyl-3-pyridinyl)methanol (56 mg, 0.277 mmol) in DCM (2 ml), MnO₂ (193 mg, 2.217 mmol) was added. After stirring overnight, an excess of MnO₂ (120 mg, 1.386 mmol) was added and then the mixture was stirred for 3 hours more. The reaction mixture was filtrated and evaporated to afford 30 mg of title compound pure enough to use in the next step.

¹H-NMR(δ, ppm, CDCl₃): 10.05 (s, 1H), 8.87 (s, 1H), 8.25 (s, 1H), 2.75 (s, 3H).

Intermediate 70.

### 5-Fluoro-6-methyl-3-pyridinecarbaldehyde (Used to Prepare Example 128)

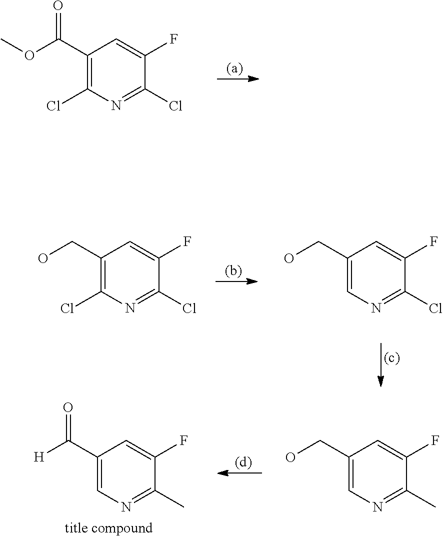

### (a) (2,6-Dichloro-5-fluoro-3-pyridinyl)methanol

To a solution of methyl 2,6-dichloro-5-fluoro-3-pyridinecarboxylate (3 g, 13.39 mmol) in DCM (12 ml) at 0° C. under N2 was added dropwise DIBAL-H (1.5 M solution in toluene, 19.20 ml, 28.8 mmol). The reaction mixture was stirred at 0° C. for 20 h. TLC (DCM) showed starting material remaining. Then, more DIBAL-H (1.5 M solution in toluene, 10 ml) was added. The reaction was stirred at 0° C. for 20 h. The reaction mixture was diluted with MeOH and concentrated under reduced pressure. The residue was treated with 1N HCl solution and extracted 3 times with EtOAc. The combined organic phases were washed with sat. NaCl, dried over Na₂SO₄ and concentrated to give 1.5 g (53%) of the title compound.

1H-NMR (δ, ppm, CDCl₃): 7.77 (d, 1H); 4.77 (bs, 2H)

### (b) (6-Chloro-5-fluoro-3-pyridinyl)methanol

To a solution of (2,6-dichloro-5-fluoro-3-pyridinyl)methanol (4.4 g, 22.45 mmol) in 1,4-dioxane (20 ml) was added Et₃N (3.42 ml, 24.69 mmol), formic acid (0.947 ml, 24.69 mmol) and Pd(PPh₃)₄ (1.297 g, 1.122 mmol). The mixture was then heated at 110° C. for 6 h in a pressure tube. The crude was diluted with H₂O and extracted twice with EtOAc. The combined organic phases were washed with H₂O and sat. NaCl, dried over Na₂SO₄ and concentrated under reduced pressure. The crude was purified by flash chromatography using Flashmaster II, a 70 g spherical silica gel cartridge and DCM/MeOH 98:2 as eluent to give 2.0 g (41%) of the title compound pure enough to be used in the next step.

1H-NMR (δ, ppm, CDCl₃): 8.18 (d, 1H); 7.70 (dd, 1H); 4.78 (bs, 2H); 1.93 (bs, 1H) [ES MS] m/z 162 (MH⁺)

### (c) (5-Fluoro-6-methyl-3-pyridinyl)methanol

To a solution of (6-chloro-5-fluoro-3-pyridinyl)methanol (1.3 g, 8.05 mmol) in 1,4-dioxane (10 ml) was added K₂CO₃ (3.34 g, 24.14 mmol), trimethylboroxin (1.125 ml, 8.05 mmol) and Pd(PPh₃)₄ (0.465 g, 0.402 mmol). The mixture was then heated at 110° C. for 20 h in a pressure tube. The resulting mixture was quenched with H2O, extracted with AcOEt, dried over MgSO₄ and concentrated under vacuum. The residue was purified by flash chromatography using Flashmaster II, a 25 g spherical silica gel cartridge and DCM/MeOH 98:2 as eluent to give 560 mg of the title compound along with Ph₃PO. This was washed again with water and extracted with AcOEt to remove Ph₃PO. The organic layer was dried over MgSO₄, filtered and concentrated under vacuum to give 468 mg (39%) of the title compound pure enough to be used in the next step.

1H-NMR (δ, ppm, CDCl₃): 8.27 (d, 1H); 7.55 (dd, 1H); 4.74 (bs, 2H); 2.49 (s, 3H) [ES MS] m/z 142 (MH⁺)

### (d) Title Compound

To a solution of (5-fluoro-6-methyl-3-pyridinyl)methanol (468 mg, 3.32 mmol) in DCM (4 ml) was added CrO₂ (Magtrieve) (4.2 g, 49.7 mmol). The mixture was stirred at 35° C. for 4 h. TLC (5% MeOH in DCM) showed starting material remaining. An excess of CrO₂ (Magtrieve) (2.8 g, 33.3 mmol) was added. The mixture was stirred at 35° C. for 48 h. The solids were filtered off and the solvent evaporated to afford 144 mg (28%) of the title compound.

1H-NMR (δ, ppm, CDCl₃): 10.32 (s, 1H); 8.57 (d, 1H); 7.81 (dd, 1H); 2.87 (s, 3H) [ES MS] m/z 140 (MH⁺).

Intermediate 71.

### 5,6-Dichloro-3-pyridinecarbaldehyde (Used to Prepare Example 129)

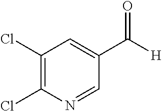

To a solution of (5,6-dichloro-3-pyridinyl)methanol (200 mg) in CH₂Cl₂, molecular sieves (3 Å) (55 mg) and 4-methy-morpholine-N-oxide (198 mg) was added and stirred at room temperature for 10 minutes. Then tetra-n-propylammonium perruthenate(VII) (38 mg) was added and stirred overnight. The reaction mixture was filtered and the solvent was evaporated to dryness under vacuum. The residue obtained was purified by chromatography column on silica gel using a mixture of EtOAc/hexane as eluent to give 44 mg (22%) of the title compound.

¹H-NMR (δ, ppm, CDCl₃): 10.09 (s, 1H), 8.77 (s, 1H), 8.25 (s, 1H).

### EXAMPLES

### Example 1

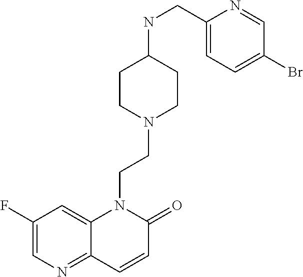

### 1-[2-(4-{[(5-Bromo-2-pyridinyl)methyl]amino}-1-piperidinyl)ethyl]-7-fluoro-1,5-naphthyridin-2(1H)-one

To a solution of Intermediate 21 (662 mg) in 1,2-dichloroethane (20 ml) was added 5-bromo-2-pyridinecarbaldehyde (404 mg, from Alfa Aesar). After stirring for a few minutes, NaBH(OAc)₃ (sodium triacetoxyborohydride) (1.45 g) was added. The reaction was stirred at room temperature overnight and more NaBH(OAc)₃ was added in several portions until starting material was not detected by LCMS. The solvent was removed to dryness under vacuum and the residue was dissolved in CH₂Cl₂. The mixture was washed with saturated NaHCO₃, brine, dried over Na₂SO₄, and concentrated under vacuum. The residue obtained was purified by column chromatography on silica gel using CH₂Cl₂/MeOH 9:1 as eluent to give 490 mg (49%) of the title compound. ¹H-NMR (δ, ppm, CDCl₃): 8.60 (d, 1H), 8.42 (d, 1H), 7.88 (d, 1H), 7.76 (dd, 1H), 7.56 (dd, 1H), 7.24 (d, 1H), 6.85 (d, 1H), 4.32 (t, (d, 1H), 4.32 (t, 2H), 3.89 (s, 2H), 2.97 (bd, 2H), 2.64 (t, 2H), 2.57-2.46 (m, 1H), 2.18 (bt, 2H), 1.92 (bd, 2H), 1.51-1.34 (m, 2H). [ES MS] m/z 460 (MH+).

### Example 1b

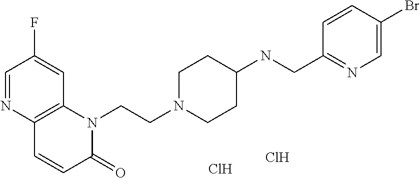

### 1-[2-(4-{[(5-Bromo-2-pyridinyl)methyl]amino}-1-piperidinyl)ethyl]-7-fluoro-1,5-napthyridin-2(1H)-one dihydrochloride

To a solution of Example 1 (24 mg) in CH₂Cl₂ (1.5 ml) was added dropwise a solution of HCl (4M solution in 1,4-dioxane, 26 μl). The mixture was stirred at room temperature for 10 minutes and the solvent was concentrated under vacuum to give the desired product (28 mg). ¹H-NMR (δ, ppm, DMSO-d₆): 10.94-10.64 (m, 1H), 9.81-9.64 (m, 1H), 8.78 (s, 1H), 8.59 (s, 1H), 8.37-8.27 (m, 1H), 8.19-8.16 (m, 1H), 7.97 (d, 1H), 7.58 (d, 1H), 6.85 (d, 1H), 4.64-4.45 (m, 2H), 4.33 (s, 2H), 3.85-3.63 (m, 1H), 3.11-2.93 (m, 2H), 2.26-1.90 (m, 4H). [ES MS] m/z 460 (MH+).

### Example 2

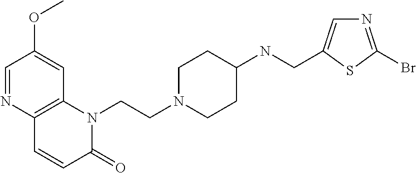

### 1-[2-(4-{[(2-Bromo-1,3-thiazol-5-yl)methyl]amino}-1-piperidinyl)ethyl]-7-(methyloxy)-1,5-naphthyridin-2(1H)-one

To a solution of Intermediate 29 (210 mg) in 1,2-dichloroethane (8 ml) was added 2-bromo-1,3-thiazole-5-carbaldehyde (80 mg, from COMBI-BLOCKS). After stirring for a few minutes, sodium triacetoxyborohydride (278 mg) was added. The reaction was stirred at room temperature overnight and more NaBH(OAc)₃ was added in several portions until starting material was not detected by LCMS. The solvent was removed to dryness under vacuum and the residue was dissolved in CH₂Cl₂. The mixture was washed with saturated NaHCO₃, brine, dried over Na₂SO₄, and concentrated under vacuum. The residue obtained was purified by column chromatography on silica gel using CH₂Cl₂/MeOH 9:1 as eluent to give 110 mg (55%) of the title compound. ¹H-NMR (δ, ppm, CDCl₃): 8.28 (d, 1H), 7.84 (d, 1H), 7.38 (s, 1H), 7.20 (s, 1H), 6.74 (d, 1H), 4.36 (t, 2H), 3.99 (bd, 2H), 3.98 (s, 3H), 2.97 (bd, 2H), 2.65 (t, 2H), 2.60-2.48 (m, 1H), 2.19 (bt, 2H), 1.89 (bd, 2H), 1.47-1.30 (m, 2H). [ES MS] m/z 478 (MH+).

### Example 2b

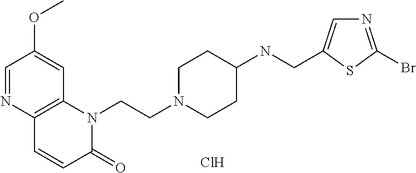

### 1-[2-(4-{[(2-Bromo-1,3-thiazol-5-yl)methyl]amino}-1-piperidinyl)ethyl]-7-(methyloxy)-1,5-naphthyridin-2(1H)-one hydrochloride

To a solution of Example 2 (83 mg) in CH₂Cl₂ (1.5 ml) was added dropwise a solution of HCl (4M solution in 1,4-dioxane, 87 μl). The mixture was stirred at room temperature for 10 minutes and the solvent was concentrated under vacuum to give the desired product (87.5 mg). ¹H-NMR (δ, ppm, DMSO-d₆): 8.30-8.29 (m,1H), 7.88 (d, 1H), 7.75-7.72 (m, 1H), 7.47-7.46 (m, 1H), 6.67 (d, 1H), 4.51-4.41 (m, 2H), 4.29-4.22 (m, 2H), 4.00 (s, 3H), 3.25-3.18 (m, 2H), 2.89-2.72 (m, 2H), 2.35-2.26 (m, 1H), 2.06-1.93 (m, 2H), 1.57-1.44 (m, 2H). [ES MS] m/z 478 (MH+).

### Example 3

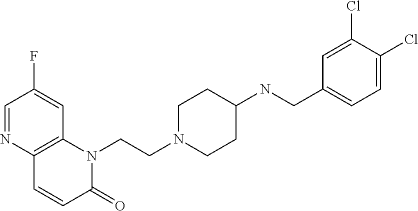

### 1-[2-(4-{[(3,4-Dichlorophenyl)methyl]amino}-1-piperidinyl)ethyl]-7-fluoro-1,5-naphthyridin-2(1H)-one

To a solution of Intermediate 21 (700 mg) in 1,2-dichloroethane (30 ml) was added 3,4-dichlorobenzaldehyde (401 mg, from Fluka). After stirring for a few minutes, sodium triacetoxyborohydride (1.5 g) was added. The reaction was stirred at room temperature overnight. The solvent was removed to dryness under vacuum and the residue was dissolved in CH₂Cl₂. The mixture was washed with saturated NaHCO₃, brine, dried over Na₂SO₄, and concentrated under vacuum. The residue obtained was purified by column chromatography on silica gel using CH₂Cl₂/MeOH 9:1 as eluent to give 495 mg of the title compound. ¹H-NMR (δ, ppm, DMSO-d₆): 8.55 (d, 1H), 8.00-7.91 (m, 2H), 7.58-7.52 (m, 2H), 7.32-7.29 (m, 1H), 6.82 (d, 1H), 4.29 (t, 2H), 3.68 (s, 2H), 2.88-2.84 (m, 2H), 2.34-2.24 (m, 1H), 2.02-1.95 (m, 2H), 1.74-1.70 (m, 2H), 1.22-1.09 (m, 2H). [ES MS] m/z 449 (MH+).

### Example 3b

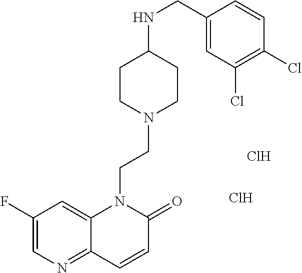

### 1-[2-(4-{[(3,4-Dichlorophenyl)methyl]amino}-1-piperidinyl)ethyl]-7-fluoro-1,5-naphthyridin-2(1H)-one dihydrochloride

To a solution of Example 3 (468 mg) in CH₂Cl₂ (10 ml) was added dropwise a solution of HCl (4M solution in 1,4-dioxane, 520 μl). The mixture was stirred at room temperature for 10 minutes and the solvent was concentrated under vacuum to give the desired product (543 mg). ¹H-NMR (δ, ppm, DMSO-d₆): 10.90-10.78 (m, 1H), 9.76-9.66 (m, 1H), 8.60 (s, 1H), 840-8.26 (m, 1H), 8.00-7.94 (m, 2H), 7.73 (d, 1H), 7.60-7.57 (m, 1H), 6.87 (d, 1H), 4.65-4.55 (m, 2H), 4.24-4.16 (m, 2H), 3.84-3.72 (m, 2H), 3.19-3.03 (m, 2H), 2.43-2.32 (m, 2H), 2.13-1.98 (m, 2H). [ES MS] m/z 449 (MH+).

### Example 4

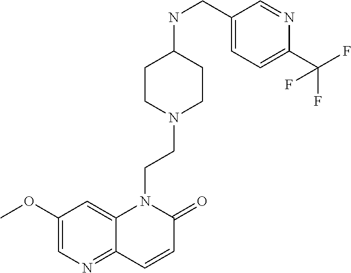

### 7-(Methyloxy)-1-{2-[4-({[6-(trifluoromethyl)-3-pyridinyl]methyl}amino)-1-piperidinyl]ethyl}-1,5-naphthyridin-2(1H)-one

To a solution of Intermediate 28 (100 mg) and Et₃N (0.1 ml) in a mixture of CH₂Cl₂ (3 ml) and MeOH (3 ml) was added 6-(trifluoromethyl)-3-pyridinecarbaldehyde (42 mg, from Aldrich). After stirring for 30 minutes, sodium triacetoxyborohydride (167 mg) was added. The resulting mixture was stirred at room temperature and more NaBH(OAc)₃ (668 mg) was added in several portions until starting material was not detected by LCMS. The solvent was removed to dryness under vacuum and the residue was dissolved in CH₂Cl₂. The mixture was washed with saturated NaHCO₃, dried over Na₂SO₄, and concentrated under vacuum. The residue obtained was purified by column chromatography on silica gel using CH₂Cl₂/MeOH 9:1 as eluent to give 15 mg of the title compound. ¹H-NMR (δ, ppm, DMSO-d₆): 8.69 (s, 1H), 8.26 (d, 1H), 8.02 (d, 1H), 7.87-7.81 (m, 2H), 7.40 (d, 1H), 6.65 (d, 1H), 4.34 (t, 2H), 3.96 (s, 3H), 3.82 (s, 2H), 2.92-2.89 (m, 2H), 2.37-2.30 (m, 1H), 2.05-1.98 (m, 2H), 1.79-1.75 (m, 2H), 1.27-1.15 (m, 2H). [ES MS] m/z 462 (MH+).

### Example 4b

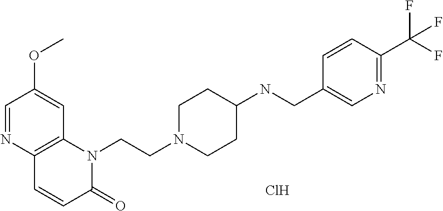

### 7-(Methyloxy)-1-{2-[4-({[6-(trifluoromethyl)-3-pyridinyl]methyl}amino)-1-piperidinyl]ethyl}-1,5-naphthyridin-2(1H)-one hydrochloride

To a solution of Example 4 (76 mg) in CH₂Cl₂ (1.5 ml) was added dropwise a solution of HCl (4M solution in 1,4-dioxane, 82 μl). The mixture was stirred at room temperature for 10 minutes and the solvent was concentrated under vacuum to give the desired product (78 mg). ¹H-NMR (δ, ppm, DMSO-d₆): 8.30-8.29 (m,1H), 7.88 (d, 1H), 7.75-7.72 (m, 1H), 7.47-7.46 (m, 1H), 6.67 (d, 1H), 4.51-4.41 (m, 2H), 4.29-4.22 (m, 2H), 4.00 (s, 3H), 3.25-3.18 (m, 2H), 2.89-2.72 (m, 2H), 2.35-2.26 (m, 1H), 2.06-1.93 (m, 2H), 1.57-1.44 (m,2H). [ES MS] m/z 478 (MH+).

### Example 5

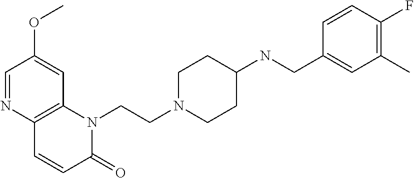

### 1-[2-(4-{[(4-Fluoro-3-methylphenyl)methyl]amino}-1-piperidinyl)ethyl]-7-(methyloxy)-1,5-naphthyridin-2(1H)-one

To a solution of Intermediate 29 (105 mg) in a mixture of CH₂Cl₂ (2.5 ml) and MeOH (2.5 ml) was added Intermediate 61 (48 mg). After stirring for a few minutes, sodium triacetoxyborohydride (223 mg) was added. The mixture was stirred at room temperature overnight and more Intermediate 61 (30 mg), NaBH(OAc)₃ and a few drops of acetic acid were added in several portions until starting material was not detected by LCMS. The resulting mixture was quenched with saturated NaHCO₃, extracted with CH₂Cl₂, dried over Na₂SO₄ and concentrated under vacuum. The residue obtained was purified by preparative HPLC (gradient elution: 10 to 100% CH₃CN/H₂O 0.1% TFA, uv detection 254 nm) to give the title compound as a salt. The obtained compound was dissolved in aqueous 10% Na₂CO₃ and extracted with CH₂Cl₂ to give 20 mg of the title compound. ¹H-NMR (δ, ppm, CDCl₃): 8.28 (d, 1H), 7.84 (d, 1H), 7.24 (bd, 1H), 7.14-7.05 (m, 2H), 6.93 (t, 1H), 6.74 (d, 1H), 4.37 (d, 1H), 3.97 (s, 3H), 3.73 (s, 2H), 2.99 (bd, 2H), 2.64 (t, 2H), 2.58-2.48 (m, 1H), 2.26 (bd, 3H), 2.19 (bt, 2H), 1.91 (bd, 2H), 1.49-1.36 (m, 2H). [ES MS] m/z 425 (MH+).

### Example 6

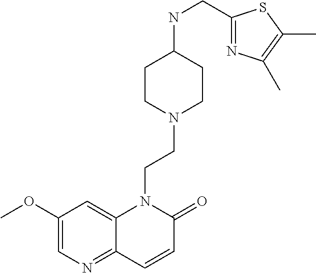

### 1-[2-(4-{[(4,5-Dimethyl-1,3-thiazol-2-yl)methyl]amino}-1-piperidinyl)ethyl]-7-(methyloxy)-1,5-naphthyridin-2(1H)-one

To a solution of Intermediate 29 (100 mg) in tetrahydrofurane (5 ml) was added Intermediate 56 (57 mg). After stirring for 1 h, sodium triacetoxyborohydride (76 mg) was added. The mixture was stirred at room temperature overnight. LCMS showed that reaction did not take place. The solvent was evaporated to dryness under vacuum, and 5 ml of 1,2-dichloroethane and sodium triacetoxyborohydride (152 mg) were added leaving the reaction under stirring overnight. The solvent was evaporated to dryness under vacuum. H₂O/NaHCO₃ 10% was added and the aqueous layer was extracted with CH₂Cl₂, dried over Na₂SO₄, concentrated under vacuum and purified by column chromatography on silica gel using CH₂Cl₂ /MeOH 80:20 as eluent to give 33 mg of the title compound as a yellow solid. ¹H-NMR (δ, ppm, CDCl₃): 8.27 (s, 1H), 7.84 (d, 1H), 6.63 (d, 1H), 4.37 (t, 2H), 4.02 (s, 2H), 3.98 (s, 3H), 3.04-2.90 (m, 2H), 2.71-2.51 (m, 3H), 2.32 (s, 3H), 2.29 (s, 3H), 2.20 (t, 2H), 2.00-1.85 (m, 2H), 1.53-1.34 (m, 2H). [ES MS ] m/z 428 (MH+).

### Example 7

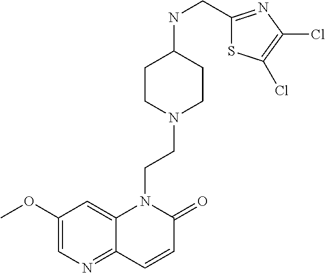

### 1-[2-(4-{[(4,5-Dichloro-1,3-thiazol-2-yl)methyl]amino}-1-piperidinyl)ethyl]-7-(methloxy)-1,5-naphthyridin-2(1H)-one

To a solution of Intermediate 29 (128 mg) in 1,2-dichloroethane (10 ml) was added Intermediate 41 (76 mg). After stirring for 30 minutes, sodium triacetoxyborohydride (293 mg) was added. The resulting mixture was stirred at room temperature for 2 h and 293 mg of NaBH(OAc)₃ were added. The mixture was stirred at room temperature overnight. The solvent was removed to dryness under vacuum, H₂O was added and pH was adjusted to 11 with aqueous NaOH. CH₂Cl₂ was added to the reaction mixture and organic phase was extracted. The residue obtained was purified by preparative HPLC. The obtained compound was dissolved in 0.5N NaOH and extracted with CH₂Cl₂ to give 32 mg of the title compound as a white solid. ¹H-NMR (δ, ppm, CDCl₃): 8.28 (d, 1H), 7.84 (d, 1H), 7.19 (d, 2H), 6.74 (d, 1H), 4.36 (t, 2H), 4.02 (d, 2H), 3.98 (s, 3H), 3.05-2.91 (m, 2H), 2.65 (t, 2H), 2.60-2.49 (m, 1H), 2.19 (dt, 2H), 1.91 (bd, 2H), 1.49-1.32 (m, 2H). [ES MS] m/z 468 (MH+).

### Example 8

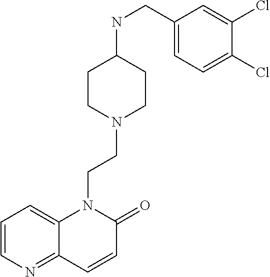

### 1-[2-(4-{[(3,4-Dichlorophenyl)methyl]amino}-1-piperidinyl)ethyl]-1,5-naphthyridin-2(1H)-one

To a solution of Intermediate 8 (1.5 g) in a mixture of CH₂Cl₂ (10 ml) and MeOH (10 ml) was added 3,4-dichlorobenzaldehyde (965 mg, from Fluka). After stirring for 30 minutes, sodium triacetoxyborohydride (3.5 g) was added. The mixture was stirred at room temperature for 3 h. The resulting mixture was quenched with saturated NaHCO₃, extracted with CH₂Cl₂, dried over Na₂SO₄ and concentrated under vacuum. The residue obtained was purified by column chromatography on silica gel using a MeOH and CH₂Cl₂ gradient (0-20%) to give the desired product as a white solid (1.58 g, 67%). ¹H-NMR (δ, ppm,CDCl₃): 8.55 (dd, 1H), 7.91 (d, 1H), 7.78 (d, 1H), 7.40 (m, 2H), 7.17 (dd, 1H), 6.91 (d, 1H), 4.38 (t, 2H), 3.76 (s, 2H), 2.97 (d, 2H), 2.64 (t, 2H), 2.50 (m, 1H), 2.18 (t, 2H), 1.87 (d, 2H), 1.42 (m, 4H). [ES MS] m/z 431 (MH+).

### Example 8b

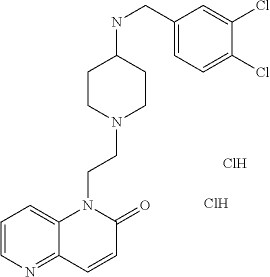

### 1-[2-(4-{[(3,4-Dichlorophenyl)methyl]amino}-1-piperidinyl)ethyl]-1,5-naphthyridin-2(1H)-one dihydrochloride

To a solution of Example 8 (940 mg) in CH₂Cl₂ (20 ml) was added dropwise a solution of HCl (4M solution in 1,4-dioxane, 1.1 ml). The mixture was stirred at room temperature for 10 minutes and the solvent was concentrated under vacuum to give the desired product (1.04 g). ¹H-NMR (δ, ppm, DMSO-d₆): 10.97-10.85 (m, 1H), 9.82-9.67 (m, 2H), 8.56 (d, 1H), 8.32-8.16 (m, 1H), 7.99-7.94 (m, 2H), 7.74-7.58 (m, 3H), 6.90 (d, 1H), 4.68-4.52 (m, 2H), 4.19 (s, 2H), 3.83-3.66 (m, 1H), 3.21-3.04 (m, 2H), 2.40-1.90 (m, 4H). [ES MS] m/z 431 (MH+).

### Example 9

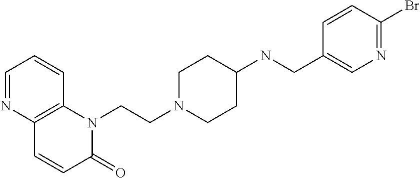

### 1-[2-(4-{[(6-Bromo-3-pyridinyl)methyl]amino}-1-piperidinyl)ethyl]-1,5-naphthyridin-2(1H)-one

To a solution of Intermediate 8 (60 mg) in 1,2-dichloroethane (5 ml) was added 6-bromo-3-pyridinecarbaldehyde (39 mg, from Aldrich) and sodium triacetoxyborohydride (140 mg). The resulting mixture was stirred at room temperature and more NaBH(OAc)₃ was added in several portions until starting material was not detected by LCMS. The solvent was removed to dryness under vacuum and the residue was dissolved in CH₂Cl₂. The mixture was washed with saturated NaHCO₃, dried over Na₂SO₄, and concentrated under vacuum. The residue obtained was purified by preparative HPLC (gradient elution: 10 to 100% CH₃CN/H₂O, X-terra 19x150 mm, uv detection 254 nm) to give 42 mg (43%) of the title compound. ¹H-NMR (δ, ppm, CDCl₃): 8.54 (d, 1H), 7.91 (d, 1H), 7.80 (m, 1H), 7.47 (dd, 1H), 7.10 (s, 1H), 6.91 (d, 1H), 6.83 (s, 1H), 4.40 (t, 2H), 3.97 (s, 2H), 2.98 (m, 2H), 2.66 (m, 2H), 2.21 (m, 2H), 1.90 (bd, 2H), 1.25-1.70 (m, 4H). [ES MS] m/z 447 (MH+).

### Example 10

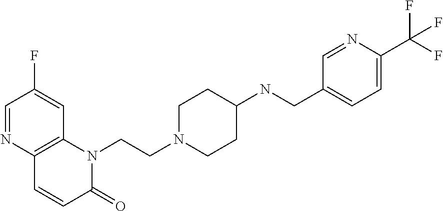

### 7-Fluoro-1-{2-[4-({[6-(trifluoromethyl)-3-pyridinyl]methyl}amino)-1-piperidinyl]ethyl}-1.5-naphthyridin-2(1H)-one

To a solution of Intermediate 20 (90 mg) and Et₃N (0.1 ml) in a mixture of CH₂Cl₂ (5 ml) and MeOH (1 ml) was added 6-(trifluoromethyl)-3-pyridinecarbaldehyde (41 mg, from Apollo). After stirring for 30 minutes, sodium triacetoxyborohydride (150 mg) was added. The resulting mixture was stirred at room temperature and more NaBH(OAc)₃ (600 mg) and a few drops of acetic acid were added in several portions until starting material was not detected by LCMS. The solvent was evaporated to dryness under vacuum, H₂O was added and pH was adjusted to 11 with 30% aqueous ammonia. The aqueous layer was extracted with EtOAc, dried over Na₂SO₄, concentrated under vacuum and purified by column chromatography on silica gel using CH₂Cl₂/MeOH 9:1 as eluent to give 45 mg (44%) of the title compound as a white solid. ¹H-NMR (δ, ppm, CDCl₃): 8.67 (s, 1H), 8.42 (d, 1H), 7.88 (d, 2H), 7.64 (d, 1H), 7.53 (bd, 1H), 6.86 (d, 1H), 4.32 (t, 2H), 3.92 (s, 2H), 2.97 (bd, 2H), 2.66 (t, 2H), 2.56-2.49 (m, 1H), 2.19 (t, 2H), 1.92 (bd, 2H), 1.46-1.36 (m, 2H). [ES MS] m/z 450 (MH+).

### Example 11

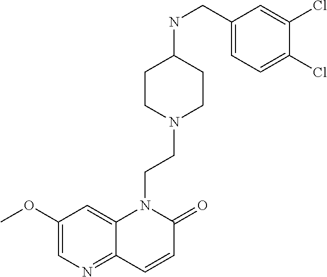

### 1-[2-(4-{[(3,4-Dichlorophenyl)methyl]amino}-1-piperidinyl)ethyl]-7-(methyloxy)-1,5-naphthyridin-2(1H)-one

To a solution of Intermediate 28 (189 mg) and Et₃N (0.2 ml) in a mixture of CH₂Cl₂ (6 ml) and MeOH (0.6 ml) was added 3,4-dichlorobenzaldehyde (76 mg, from Fluka). After stirring for 30 minutes, sodium triacetoxyborohydride (300 mg) was added. The resulting mixture was stirred at room temperature and more NaBH(OAc)₃ (900 mg) was added in several portions until starting material was not detected by LCMS. The resulting mixture was quenched with saturated NaHCO₃, extracted with a mixture of CH₂Cl₂/MeOH 8:2, dried over Na₂SO₄ and concentrated under vacuum. The residue obtained was purified by column chromatography on silica gel using CH₂Cl₂/MeOH 95:5 as eluent to give 82 mg (40%) of the title compound as a white solid. ¹H-NMR (δ, ppm, CDCl₃): 8.28 (d, 1H), 7.84 (d, 1H), 7.44 (s, 1H), 7.38 (d, 2H), 7.25 (bd, 1H), 7.16 (d, 1H), 6.73 (d, 1H), 4.38 (t, 2H), 3.98 (s, 3H), 3.77 (s, 2H), 2.99 (bd, 2H), 2.66 (t, 2H), 2.58-2.45 (m, 1H), 2.28-2.15 (m, 2H), 1.95 (d, 2H), 1.50-1.35 (m, 2H). ES MS] m/z 461 (MH+).

### Example 11b

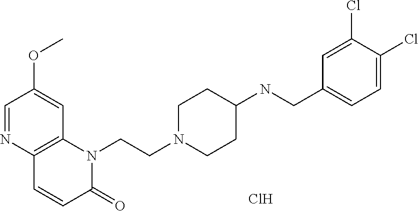

### 1-[2-(4-{[(3,4-Dichlorophenyl)methyl]amino}-1-piperidinyl)ethyl]-7-(methyloxy)-1,5-naphthyridin-2(1H)-one hydrochloride

To a solution of Example 11 (14.3 mg) in CH₂Cl₂ (0.5 ml) was added dropwise a solution of HCl (4M solution in 1,4-dioxane, 15.5 ₁1.1). The mixture was stirred at room temperature for 10 minutes and the solvent was concentrated under vacuum to give the desired product (12 mg). ¹H-NMR (δ, ppm, DMSO-d₆): 8.28 (d, 1H), 7.88-7.83 (m, 2H), 7.70-7.68 (m, 1H), 7.51-7.48 (m, 1H), 7.42 (s, 1H), 6.65 (d, 1H), 4.40-4.36 (m, 2H), 4.11-4.06 (m, 2H), 3.98 (s, 3H), 3.11-3.04 (m, 2H), 2.97-2.83 (m, 1H), 2.61-2.57 (m, 2H), 2.13-1.97 (m, 4H), 1.50-1.45 (m, 2H). [ES MS] m/z 461 (MH+).

### Example 11c

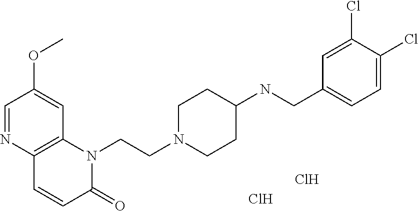

### 1-[2-(4-{[(3,4-Dichlorophenyl)methyl]amino}-1-piperidinyl)ethyl]-7-(methyloxy)-1,5-naphthyridin-2(1H)-one dihvdrochloride

To a solution of Example 11 (230 mg) in CH₂Cl₂ (5 ml) was added dropwise a solution of HCl (4M solution in 1,4-dioxane, 249 μl). The mixture was stirred at room temperature for 10 minutes and the solvent was concentrated under vacuum to give the desired product (259 mg).

¹H-NMR (δ, ppm, DMSO-d₆): 11.32-11.18 (m, 1H), 9.75-9.56 (m, 2H), 8.31 (d, 1H), 7.93-7.90 (m, 2H), 7.74-7.57 (m, 3H), 6.69 (d, 1H), 4.72-4.58 (m, 2H), 4.24-4.16 (m, 2H), 4.04 (s, 3H), 3.86-3.65 (m, 2H), 3.27-3.02 (m, 3H), 2.43-1.93 (m, 4H). [ES MS] m/z 461 (MH+).

### Example 12

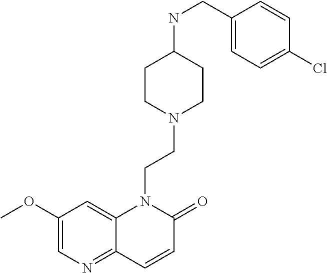

### 1-[2-(4-{[(4-Chlorophenyl)methyl]amino}-1-piperidinyl)ethyl]-7-(methyloxy)-1,5-naphthyridin-2(1H)-one

To a solution of Intermediate 28 (100 mg) and Et₃N (0.1 ml) in a mixture of CH₂Cl₂ (5 ml) and MeOH (1 ml) was added 4-chlorobenzaldehyde (34 mg, from Fluka). After stirring for 30 minutes, sodium triacetoxyborohydride (167 mg) was added. The resulting mixture was stirred at room temperature and more NaBH(OAc)₃ (668 mg) was added in several portions until starting material was not detected by LCMS. The resulting mixture was quenched with saturated NaHCO₃, extracted with CH₂Cl₂, dried over Na₂SO₄ and concentrated under vacuum. The residue obtained was purified by column chromatography on silica gel using CH₂Cl₂/MeOH 85:15 as eluent to give 66 mg (59%) of the title compound as a yellow solid. ¹H-NMR (δ, ppm, CDCl₃): 8.25 (d, 1H), 7.86 (d, 1H), 7.22-7.34 (m, 5H), 6.73 (d, 1H), 4.44 (t, 2H), 4.00 (s, 2H), 3.79 (s, 2H), 3.07 (bd, 2H), 2.72 (t, 2H), 2.59 (bs, 1H), 2.31 (bs, 2H), 1.96 (d, 2H), 1.41-1.60 (m, 2H). [ES MS] m/z 427 (MH+).

### Example 12b

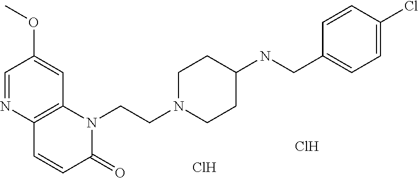

### 1-[2-(4-{[(4-Chlorophenyl)methyl]amino}-1-piperidinyl)ethyl]-7-(methyloxy)-1,5-naphthyridin-2(1H)-one dihydrochloride

To a solution of Example 12 (205 mg) in CH₂Cl₂ (5 ml) was added dropwise a solution of

HCl (4M solution in 1,4-dioxane, 240 μl). The mixture was stirred at room temperature for 10 minutes and the solvent was concentrated under vacuum to give the desired product (215 mg). ¹H-NMR (δ, ppm, DMSO-d₆): 11.14-11.07 (m, 1H), 9.57-9.48 (m, 1H), 9.10-8.96 (m, 1H), 8.30 (s, 1H), 7.91-7.90 (m, 1H), 7.57-7.50 (m, 5H), 6.69-6.67 (m, 1H), 4.71-4.63 (m, 1H), 4.43-4.34 (m, 1H), 4.21-4.12 (m, 2H), 4.01 (s, 3H), 3.83-3.70 (m, 1H), 3.17-2.98 (m, 3H), 2.17-1.96 (m, 3H), 1.62-1.47 (m, 1H). [ES MS] m/z 427 (MH+).

### Example 12c

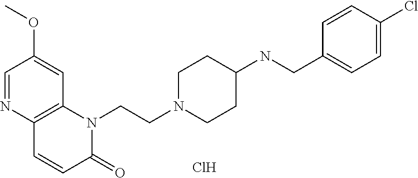

### 1-[2-(4-{[(4-Chlorophenyl)methyl]amino}-1-piperidinyl)ethyl]-7-(methyloxy)-1,5-naphthyridin-2(1H)-one hydrochloride

To a solution of Example 12 (328 mg) in CH₂Cl₂ (6 ml) was added dropwise a solution of HCl (3N solution in MeOH, 240 μl). The mixture was stirred at room temperature for 10 minutes and the solvent was concentrated under vacuum to give the desired product (215 mg). ¹H-NMR (δ, ppm, D₂O): 8.13 (d, 1H), 7.78 (d, 1H), 7.33-7.17 (m, 5H), 6.62 (d, 1H), 4.29 (bt, 2H), 4.04 (s, 2H), 3.83 (s, 3H), 3.08 (bd, 3H), 2.60 (bt, 2H), 2.18 (bt, 2H), 2.03 (bd, 2H), 1.62-1.44 (m, 2H). [ES MS] m/z 427 (MH+).

### Example 13

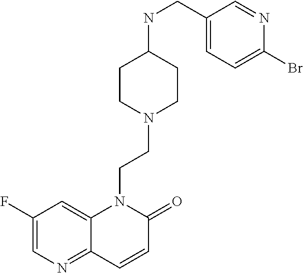

### 1-[2-(4-{[(6-Bromo-3-pyridinyl)methyl]amino}-1-piperidinyl)ethyl]-7-fluoro-1,5-naphthyridin-2(1H)-one

To a solution of Intermediate 20 (100 mg) and Et₃N (0.11 ml) in a mixture of CH₂Cl₂ (5 ml) and MeOH (1 ml) was added 6-bromo-3-pyridinecarbaldehyde (49 mg, from Aldrich). After stirring for 30 minutes, sodium triacetoxyborohydride (171 mg) was added. The resulting mixture was stirred at room temperature and more NaBH(OAc)₃ (684 mg) was added in several portions until starting material was not detected by LCMS. The solvent was evaporated to dryness under vacuum, H₂O was added and pH was adjusted to 11 with aqueous 6N NaOH. The aqueous layer was extracted with a mixture of CH₂Cl₂/MeOH 95:5, dried over Na₂SO₄, concentrated under vacuum and purified by column chromatography on silica gel using CH₂Cl₂/MeOH 95:5 as eluent to give 52 mg (45%) of the title compound as a white solid. ¹H-NMR (δ, ppm, CD₃OD): 8.48 (d, 1H), 8.32 (d, 1H), 7.92-8.00 (m, 2H), 7.72 (dd, 1H), 7.56 (d, 1H), 6.86 (d, 1H), 4.42 (t, 2H), 3.78 (s, 2H), 3.04 (d, 2H), 2.65 (t, 2H), 2.43-2.55 (m, 2H), 2.16 (t, 2H), 1.99 (d, 2H), 1.33-1.49 (m, 2H). [ES MS] m/z 460 (MH+).

### Example 14

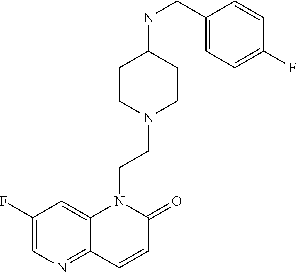

### 7-Fluoro-1-[2-(4-{[(4-fluorophenyl)methyl]amino}-1-piperidinyl)ethyl]-1,5-naphthyridin-2(1H)-one

To a solution of Intermediate 20 (90 mg) and Et₃N (0.11 ml) in a mixture of CH₂Cl₂ (5 ml) and MeOH (1 ml) was added 4-fluorobenzaldehyde (0.02 ml, from Acros). After stirring on an orbital shaker for 5 minutes, a solution of polymer-supported cyanoborohydride (242 mg, load=2.5 mmol/g) and a few drops of acetic acid were added. The mixture was stirred at room temperature overnight. The resulting solution was filtered and the resin was washed with MeOH and CH₂Cl₂. The solvent was eliminated under vacuum. The residue obtained was purified by column chromatography on silica gel using CH₂Cl₂/MeOH 9:1 as eluent to give 20 mg (22%) of the title compound as a white solid. ¹H-NMR (δ, ppm, CD₃OD): 8.50 (d, 1H), 8.01-7.95 (m, 2H), 7.55-7.50 (m, 2H), 7.23-7.17 (m, 2H), 6.88 (d, 1H), 4.49 (t, 2H), 4.21 (s, 2H), 3.29-3.18 (m, 3H), 2.87 (m, 2H), 2.38 (m, 2H), 2.18 (bd, 2H), 1.71 (m, 2H). [ES MS] m/z 399 (MH+).

### Example 15

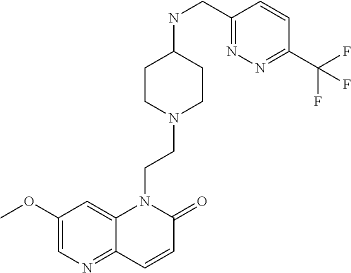

### 7-(Methyloxy)-1-{2-[4-({[6-(trifluoromethyl)-3-pyridazinyl]methyl}amino)-1-piperidinyl]ethyl}-1,5-naphthyridin-2(1H)-one

To a solution of Intermediate 29 (35.1 mg) in acetonitrile (1.5 ml) was added a mixture of K₂CO₃ (16.0 mg) and Intermediate 46 (14.0 mg). The reaction mixture was stirred at room temperature for 3 h. The solid was filtered off and the solvent evaporated. The residue obtained was purified by column chromatography on silica gel using CH₂Cl₂/MeOH as eluent to give 16 mg (60%) of the title compound as a yellow solid. ¹H-NMR (δ, ppm, CDCl₃): 8.29 (d, 1H), 7.88-7.77 (m, 3H), 7.23 (bd, 1H), 6.75 (d, 1H), 4.38 (bt, 2H), 4.26 (s, 2H), 3.99 (s, 3H), 3.07-2.96 (m, 2H), 2.73-2.53 (m, 3H), 2.22 (bt, 2H), 1.95 (bd, 2H), 1.56-1.38 (m, 2H). [ES MS] m/z 463 (MH+).

### Example 16

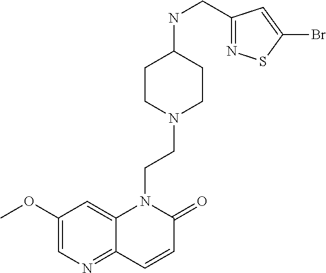

### 1-[2-(4-{[(5-Bromo-3-isothiazolyl)methyl]amino}-1-piperidinyl)ethyl]-7-(methyloxy)-1,5-naphthyridin-2(1H)-one

To a solution of Intermediate 29 (92.0 mg) in acetonitrile (2 ml) and methanol (1 ml) was added a mixture of K₂CO₃ (41.5 mg) and 5-bromo-3-(bromomethyl)isothiazole (39.2 mg, Sunshine Chemlab). The reaction mixture was stirred at room temperature for 3 h. The solid was filtered off and the solvent evaporated. The residue obtained was purified by column chromatography on silica gel using CH₂Cl₂/MeOH as eluent and further purified by trituration with a t-BuOMe/hexane 1:2 mixture to give 15.5 mg (21%) of the title compound as a beige solid. ¹H-NMR (δ, ppm, CDCl₃): 8.28 (d, 1H), 7.85 (d, 1H), 7.29-7.24 (m, 1H), 7.19 (s, 1H), 6.74 (d, 1H), 4.39 (bt, 2H), 3.99 (s, 3H), 3.95 (s, 2H), 3.09-2.95 (m, 2H), 2.67 (bt, 2H), 2.63-2.50 (m, 1H), 2.23 (bt, 2H), 1.93 (bd, 2H), 1.55-1.37 (m, 2H). [ES MS] m/z 478 and 480 (MH+).

Example 119

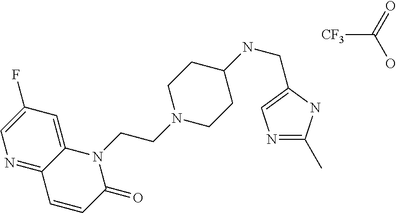

### 7-fluoro-1-[2-(4-{[(2-methyl-1H-imidazol-4-yl)methyl]amino}-1-piperidinyl)ethyl]-1,5-naphthyridin-2(1H)-one trifluoroacetate

To a suspension of 1-[2-(4-amino-1-piperidinyl)ethyl]-7-fluoro-1,5-naphthyridin-2(1H)-one (70 mg, 0.241 mmol) and 2-methyl-1H-imidazole-4-carbaldehyde (29 mmol, 0.265 mmol) in a mixture of THF/EtOH (3:1.4 ml) under nitrogene atmosphere, titanium(IV) isopropoxide (137 mg, 0.482 mmol) was added and the mixture was left under stirring over 7 h. Then sodiumborohydride (27.4 mg, 0.723 mmol) was added and the reaction was left overnight. Next morning LCMS showed the reaction was finished. The solvent was removed under vacuum and MeOH was added, corresponding precipitate was filtered and the resulting filtrate was concentrated to dryness. The crude obteined was purified by flash master II, —NH₂ gel, DCM/MeOH (0-15%) to give the desired product (48 mg) not pure enough. The residue obtained was purified by preparative HPLC (gradient elution: 10 to 100% CH₃CN/H₂O 0.1% TFA, Sunfire column), affording 11 mg (9%) of the desired compound as a trifluoroacetate. ¹H-NMR (δ, ppm, CD₃OD): 8.54 (s, 1H), 8.09-7.92 (m, 2H), 7.59 (s, 1H), 6.90 (d, 1H), 4.76-4.64 (m, 2H), 4.46 (s, 2H), 4.12-3.97 (m, 2H), 3.49-3.73 (m, 3H), 3.30-2.28 (m, 2H), 2.65 (s, 3H), 2.57-2.37 (m, 2H), 2.17-2.00 (m, 2H). [ES MS] m/z 385 (MH⁺).

### Example 131

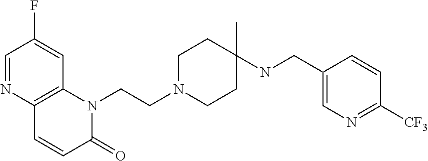

### 7-fluoro-1-{2-[4-methyl-4-({[6-(trifluoromethyl)-3-pyridinyl]methyl}amino)-1-piperidinyl]ethyl}-1,5-naphthyridin-2(1H)-one (Scheme 12)

### (a) 1,1-Dimethylethyl 4-methyl-4-({[6-(trifluoromethyl)-3-pyridinyl]methyl}amino)-1-piperidinecarboxylate

To a solution of 1,1-dimethylethyl 4-amino-4-methyl-1-piperidinecarboxylate (500 mg, 2.333 mmol) in DCM (15 ml) was added 6-(trifluoromethyl)pyridine-3-carboxaldehyde (388 mg, 2.216 mmol). The mixture was stirred at room temperature for 12 h, NaBH(OAc)₃ (1483 mg, 7.00 mmol) was added and it was stirred at room temperature for 12 h. Solvent was evaporated under vacuum and the obtained solid was dissolved in DCM. The organic phase was washed with sat NaHCO₃ and brine, dried over Na₂SO₄, filtered and concentrated. The residue was purified by column chromatography on silica gel using hexane/ethyl acetate 50:50 to give 440 mg of a mixture 7:3 desired compound and corresponding imine. The mixture was used in the next step without further purification. ¹H-NMR (δ, ppm, CDCl₃): 8.70 (s, 1H), 7.94 (d, 1H), 7,65 (d,1H), 3.82 (s, 2H), 3.47 (bs, 4H), 1.57-1.53 (m, 4H), 1.47 (s, 9H), 1.22 (s, 3H). [ES MS] m/z 374 (MH⁺).

### (b) 4-Methyl-N-{[6-(trifluoromethyl)-3-pyridinyl]methyl}-4-piperidinamine

To a solution of 1,1-dimethylethyl 4-methyl-4-({[6-(trifluoromethyl)-3-pyridinyl]methyl}amino)-1-piperidinecarboxylate (220 mg, 0.589 mmol) in DCM (4 ml) was added a solution of HCl (4M solution in 1,4-dioxane, 1.5 ml). The reaction mixture was stirred at room temperature for 4 h. The mixture was evaporated to deliver a white solid, the residue was then dissolved in methanol and placed onto a 5 g SCX column and eluted with methanol then 2 M NH₃ in methanol. The basic fractions were evaporated to deliver 142.4 mg (88%) of the desired compound as a clear colourless oil pure enough to be used in the next step.

¹H-NMR (δ, ppm, CDCl₃): 8.67 (s, 1H), 7.89 (d, 1H), 7.59 (d, 1H), 3.78 (s, 2H), 3.00-2.93 (m, 2H), 2.80-2.75 (m, 2H), 1.56-1.53 (m, 4H), 1.16 (s, 3H). [ES MS] m/z 274 (MH⁺).

### (c) Title compound: 7-Fluoro-1-{2-[4-methyl-4-({[6-(trifluoromethyl)-3-pyridinyl]methyl}amino)-1-piperidinyl]ethyl}-1,5-naphthyridin-2(1H)-one

To a solution of (7-fluoro-2-oxo-1,5-naphthyridin-1(2H)-yl)acetaldehyde (50.2 mg, 0.243 mmol) (Intermediate 18 above) in 1,2-dichloroethane (5 ml) was added 4-methyl-N-{[6-(trifluoromethyl)-3-pyridinyl]methyl}-4-piperidinamine (70,0 mg, 0,256 mmol). The mixture was stirred at room temperature for 12 h and then NaBH(OAc)₃ (163 mg, 0.769 mmol) was added. The mixture was stirred at room temperature overnight. Solvent was evaporated under vacuum and the residue was dissolved in DCM. The organic phase was washed with sat NaHCO₃ and brine, dried over Na₂SO₄, filtered and concentrated. The residue was purified by column chromatography on silica gel using DCM/MeOH 95:5 as eluent to give 67.2 mg (54%) of the title compound as a pale solid. ¹H-NMR (δ, ppm, CDCl₃): 8.70 (s, 1H), 8.42 (d, 1H), 7.93-7.87 (m, 2H), 7.63 (d, 1H), 7.55 (bd, 1H), 6.86 (d, 1H), 4.35 (t, 2H), 3.79 (s, 2H), 2.71-2.62 (m, 6H), 1.65 (bs, 4H), 1.18 (s, 3H). ([ES MS] m/z 464 (MH⁺).

### Example 132

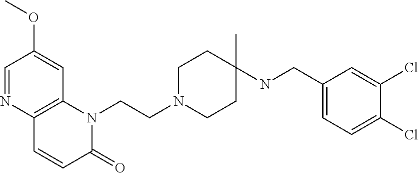

### 7-(methyloxy)-1-{2-[4-methyl-4-({[6-(trifluoromethyl)-3-pyridinyl]methyl}amino)-1-piperidinyl]ethyl}-1,5-naphthyridin-2(1H)-one (Scheme 12).

### (a) 1,1-Dimethylethyl 4-{[(3,4-dichlorophenyl)methyl]amino}-4-methyl-1-piperidinecarboxylate

To a solution of 3,4-Dichlorobenzaldehyde (388 mg, 2.216 mmol) in DCM (15 ml) was added 1,1-dimethylethyl 4-amino-4-methyl-1-piperidinecarboxylate (500 mg, 2.333 mmol) and the solution was stirred at room temperature for 12 h. Then, NaBH(OAc)₃ (1483 mg, 7.00 mmol) was added and the mixture was stirred at room temperature for 12 h. Solvent was evaporated under vacuum and the residue was dissolved in DCM. The organic phase was washed with sat NaHCO₃ and brine, dried over Na₂SO₄, filtered and concentrated. The residue was purified by column chromatography on silica gel using hexane/ethyl acetate 50:50 to give 412 mg of a mixture 7:3 desired compound and corresponding imine. The mixture was used in the next step without further purification. ¹H-NMR (□, ppm, CDCl₃): 7.48 (s, 1H), 7.44-7.36 (m, 1H), 7,21-7.18 (m,1H), 3.66 (s, 2H), 3.46 (bs, 4H), 1.53-1.43 (m, 4H), 1.46 (s, 9H), 1.18 (s, 3H). [ES MS] m/z 373 (MH⁺).

### (b) N-[(3,4-Dichlorophenyl)methyl]-4-methyl-4-piperidinamine

To a solution of 1,1-dimethylethyl 4-{[(3,4-dichlorophenyl)methyl]amino}-4-methyl-1-piperidinecarboxylate (200 mg, 0.269 mmol) in DCM (4 ml) was added a solution of HCl (4M solution in 1,4-dioxane, 1.3 ml). The reaction mixture was stirred at room temperature for 6 h. The mixture was evaporated to deliver a white solid, the residue was then dissolved in MeOH and clear yellow solution placed onto a 5 g SCX column and eluted with methanol then 2 M NH₃ in MeOH. The basic fractions were evaporated to deliver 118.5 mg of the desired compound as a clear colourless oil pure enough to be used in the next step.

¹H-NMR (δ, ppm, CDCl₃): 7.47 (bs, 1H), 7.37 (d, 1H), 7.19 (dd, 1H), 3.65 (s, 2H), 3.11-3.03 (m, 2H), 2.92-2.84 (m, 2H), 1.64-1.61 (m, 4H), 1.19 (s, 3H). [ES MS] m/z 273 (MH⁺).

### (c) Title compound: 7-(Methyloxy)-1-{2-[4-methyl-4-({[6-(trifluoromethyl)-3-pyridinyl]methyl}amino)-1-piperidinyl]ethyl}-1,5-naphthyridin-2(1H)-one

To a solution of [7-(methyloxy)-2-oxo-1,5-naphthyridin-1(2H)-yl]acetaldehyde (39.7 mg, 0.182 mmol) (Intermediate 26 above) in 1,2-dichloroethane (5 ml) was added N-[(3,4-dichlorophenyl)methyl]-4-methyl-4-piperidinamine (52.3 mg, 0.191 mmol). The mixture was stirred at room temperature for 4 h and then NaBH(OAc)₃ (122 mg, 0.574 mmol) was added. The mixture was stirred at room temperature overnight. Solvent was evaporated under vacuum and the residue was dissolved in DCM. The organic phase was washed with sat NaHCO₃ and brine, dried over Na₂SO₄, filtered and concentrated. The residue was purified by column chromatography on silica gel using DCM/MeOH 95:5 as eluent to give 74.4 mg (82%) of the title compound as a pale solid.

¹H-NMR(δ, ppm, CDCl₃): 8.29 (d, 1H), 7.85 (d, 1H), 7.47 (s, 1H), 7.37 (d, 1H), 7.27 (bs, 1H), 7.19 (dd, 1H), 6.74 (d, 1H), 4.43 (bs, 2H), 4.00 (bs, 3H), 3.65 (s, 2H), 2.70 (bs, 5H), 1.67 (bs, 3H), 1.52 (bs, 2H), 1.18 (s, 3H). ([ES MS] m/z 475 (MH⁺).

### Examples 17-135

Examples 17-135 were prepared using methods analogous to those described for the above Examples, using the starting materials as indicated in the table below.

| Ex. | Structure | chemical name |
|---|---|---|
| 17 | 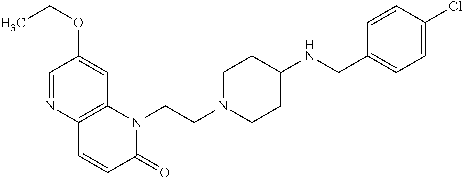 | 1-[2-(4-{[(4- chlorophenyl)methyl] amino}-1- piperidinyl)ethyl]- 7-(ethyloxy)-1,5- naphthyridin-2(1H)-one |
| 18 | 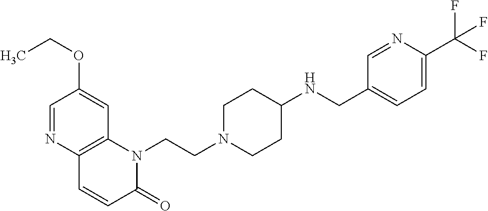 | 7-(ethyloxy)-1-{2- [4-({[6- (trifluoromethyl)-3- pyridinyl]methyl} amino)-1- piperidinyl]ethyl}- 1,5-naphthyridin- 2(1H)-one |
| 19 | 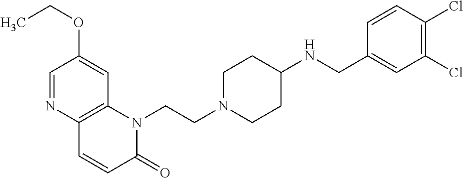 | 1-[2-(4-{[(3,4- dichlorophenyl) methyl]amino}-1- piperidinyl)ethyl]- 7-(ethyloxy)-1,5- naphthyridin- 2(1H)-one |
| 20 | 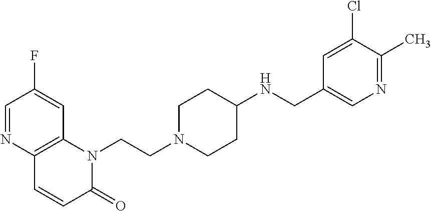 | 1-[2-(4-{[(5- chloro-6-methyl-3- pyridinyl)methyl] amino}-1- piperidinyl)ethyl]- 7-fluoro-1,5- naphthyridin- 2(1H)-one |
| 21 | 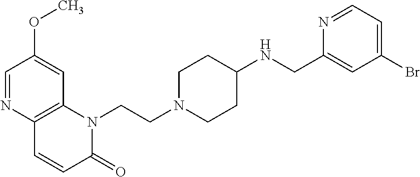 | 1-[2-(4-{[(4- bromo-2- pyridinyl)methyl] amino}-1- piperidinyl)ethyl]- 7-(methyloxy)- 1,5-naphthyridin- 2(1H)-one |
| 22 | 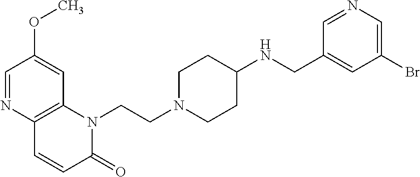 | 1-[2-(4-{[(5- bromo-3- pyridinyl)methyl] amino}-1- piperidinyl)ethyl]- 7-(methyloxy)- 1,5-naphthyridin- 2(1H)-one |
| 23 | 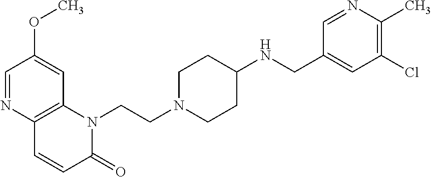 | 1-[2-(4-{[(5- chloro-6-methyl-3- pyridinyl)methyl] amino}-3- piperidinyl)ethyl]- 7-(methyloxy)- 1,5-naphthyridin- 2(1H)-one |
| 24 | 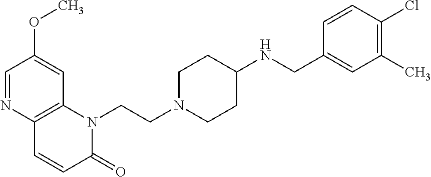 | 1-[2-(4-{[(4- chloro-3- methylphenyl) methyl]amino}-1- piperidinyl)ethyl]- 7-(methyloxy)- 1,5-naphthyridin- 2(1H)-one |
| 25 | 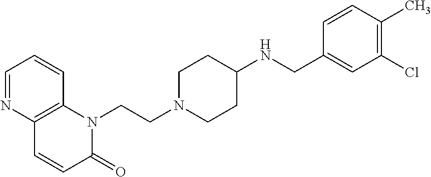 | 1-[2-(4-{[(3- chloro-4- methylphenyl) methyl]amino}-1- piperidinyl)ethyl]- 1,5-naphthyridin- 2(1H)-one |
| 26 | 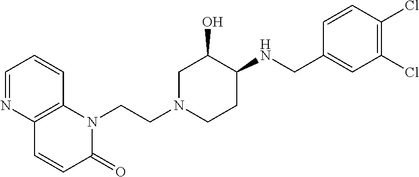 | 1-[2-((3R,4S)-4- {[(3,4- dichlorophenyl) methyl]amino}-3- hydroxy-1- piperidinyl)ethyl]- 1,5-naphthyridin- 2(1H)-one |
| 27 | 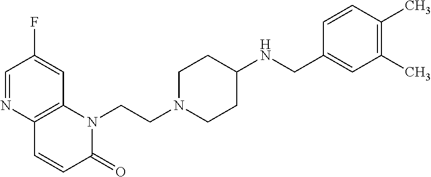 | 1-[2-(4-{[(3,4- dimethylphenyl) methyl]amino}-1- piperidinyl)ethyl]- 7-fluoro-1,5- naphthyridin- 2(1H)-one |
| 28 | 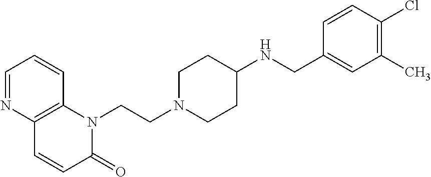 | 1-[2-(4-{[(4- chloro-3- methylphenyl) methyl]amino}-1- piperidinyl)ethyl]- 1,5-naphthyridin- 2(1H)-one |
| 29 | 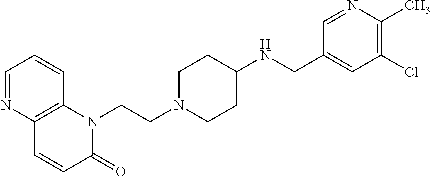 | 1-[2-(4-{[(5- chloro-6-methyl-3- pyridinyl)methyl] amino}-1- piperidinyl)ethyl]- 1,5-naphthyridin- 2(1H)-one |
| 30 | 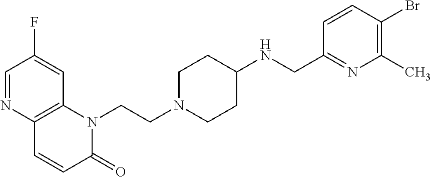 | 1-[2-(4-{[(5- bromo-6-methyl-2- pyridinyl)methyl] amino}-1- piperidinyl)ethyl]- 7-fluoro-1,5- naphthyridin- 2(1H)-one |
| 31 | 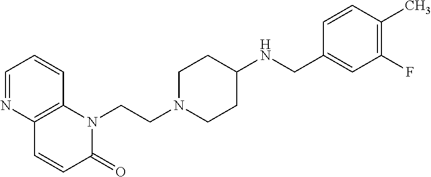 | 1-[2-(4-{[(3- fluoro-4- methylphenyl) methyl]amino}-1- piperidinyl)ethyl]- 1,5-naphthyridin- 2(1H)-one |
| 32 | 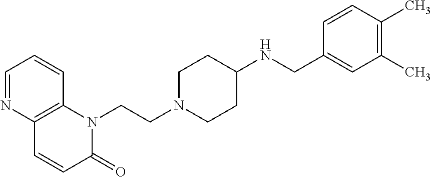 | 1-[2-(4-{[(3,4- dimethylphenyl) methyl]amino}-1- piperidinyl)ethyl]- 1,5-naphthyridin- 2(1H)-one |
| 33 | 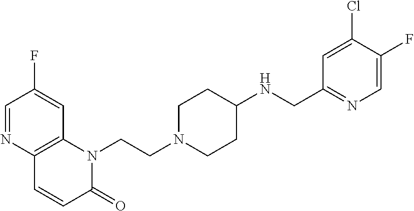 | 1-[2-(4-{[(4- chloro-5-fluoro-2- pyridinyl)methyl] amino}-1- piperidinyl)ethyl]- 7-fluoro-1,5- naphthyridin- 2(1H)-one |
| 34 | 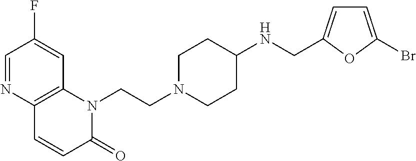 | 1-[2-(4-{[(5- bromo-2- furanyl)methyl] amino}-1- piperidinyl)ethyl]- 7-fluoro-1,5- naphthyridin- 2(1H)-one |
| 35 | 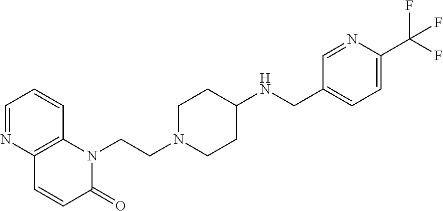 | 1-{2-[4-({[6- (trifluoromethyl)- 3-pyridinyl]methyl} amino)-1- piperidinyl]ethyl}- 1,5-naphthyridin- 2(1H)-one |
| 36 | 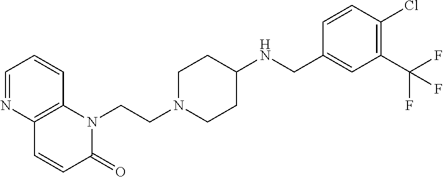 | 1-{2-[4-({[4- chloro-3- (trifluoromethyl) phenyl]methyl} amino)-1- piperidinyl]ethyl}- 1,5-naphthyridin- 2(1H)-one |
| 37 | 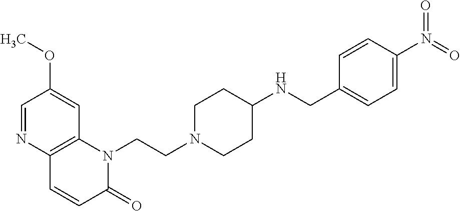 | 7-(methyloxy)-1- [2-(4-{[(4- nitrophenyl)methyl] amino}-1- piperidinyl)ethyl]- 1,5-naphthyridin- 2(1H)-one |
| 38 | 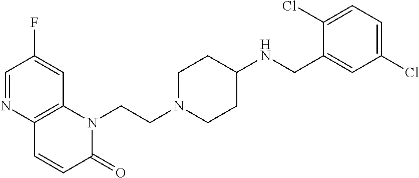 | 1-[2-(4-{[(2,5- dichlorophenyl) methyl]amino}-1- piperidinyl)ethyl]- 7-fluoro-1,5- naphthyridin- 2(1H)-one |
| 39 | 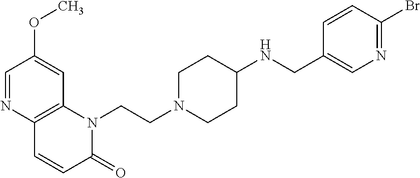 | 1-[2-(4-{[(6- bromo-3- pyridinyl)methyl] amino}-1- piperidinyl)ethyl]- 7-(methyloxy)- 1,5-naphthyridin- 2(1H)-one |
| 40 | 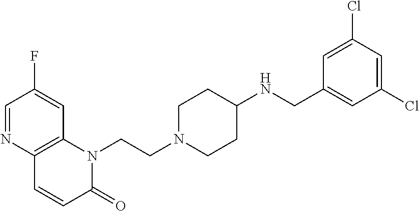 | 1-[2-(4-{[(3,5- dichlorophenyl) methyl]amino}-1- piperidinyl)ethyl]- 7-fluoro-1,5- naphthyridin- 2(1H)-one |
| 41 | 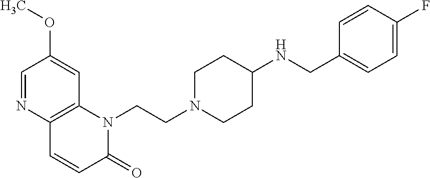 | 1-[2-(4-{[(4- fluorophenyl)methyl] amino}-1- piperidinyl)ethyl]- 7-(methyloxy)- 1,5-naphthyridin- 2(1H)-one |
| 42 | 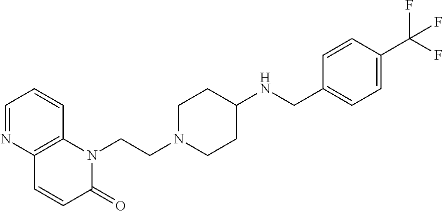 | 1-{2-[4-({[4- (trifluoromethyl) phenyl]methyl} amino)-1- piperidinyl]ethyl}- 1,5-naphthyridin- 2(1H)-one |
| 43 | 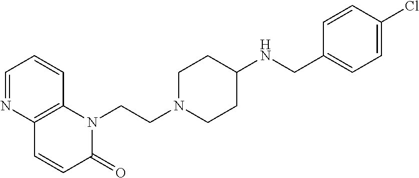 | 1-[2-(4-{[(4- chlorophenyl)methyl] amino}-1- piperidinyl)ethyl]- 1,5-naphthyridin- 2(1H)-one |
| 44 | 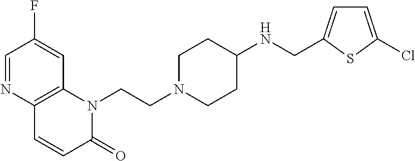 | 1-[2-(4-{[(5- chloro-2- thienyl)methyl] amino}-1- piperidinyl)ethyl]- 7-fluoro-1,5- naphthyridin- 2(1H)-one |
| 45 | 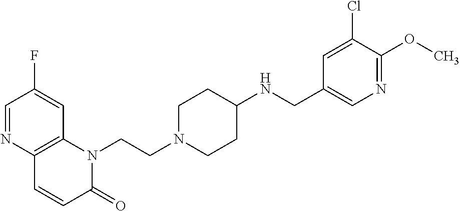 | 1-{2-[4-({[5- chloro-6- (methyloxy)-3- pyridinyl]methyl} amino)-1- piperidinyl]ethyl}- 7-fluoro-1,5- naphthyridin- 2(1H)-one |
| 46 | 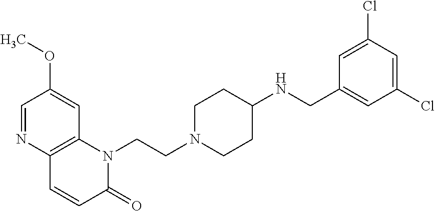 | 1-[2-(4-{[(3,5- dichlorophenyl) methyl]amino}-1- piperidinyl)ethyl]- 7-(methyloxy)- 1,5-naphthyridin- 2(1H)-one |
| 47 | 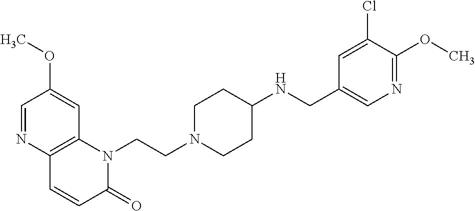 | 1-{2-[4-({[5- chloro-6- (methyloxy)-3- pyridinyl]methyl} amino)-1- piperidinyl]ethyl}- 7-(methyloxy)- 1,5-naphthyridin- 2(1H)-one |
| 48 | 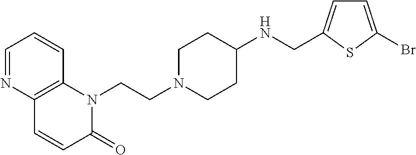 | 1-[2-(4-{[(5- bromo-2- thienyl)methyl] amino}-1- piperidinyl)ethyl]- 1,5-naphthyridin- 2(1H)-one |
| 49 | 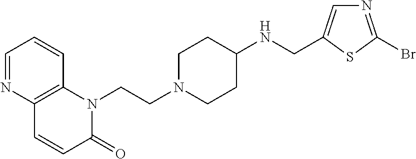 | 1-[2-(4-{[(2- bromo-1,3- thiazol-5- yl)methyl]amino}- 1-piperidinyl)ethyl]- 1,5-naphthyridin- 2(1H)-one |
| 50 | 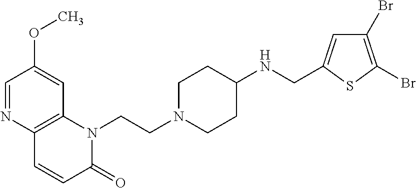 | 1-[2-(4-{[(4,5- dibromo-2- thienyl)methyl] amino}-1- piperidinyl)ethyl]- 7-(methyloxy)- 1,5-naphthyridin- 2(1H)-one |
| 51 | 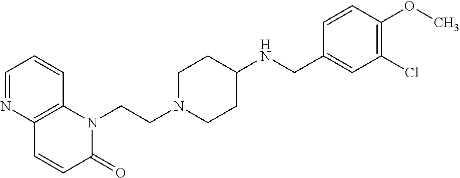 | 1-{2-[4-({[3- chloro-4- (methyloxy) phenyl]methyl}amino)- 1-piperidinyl]ethyl}- 1,5-naphthyridin- 2(1H)-one |
| 52 | 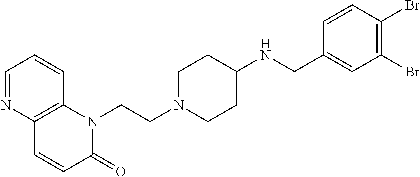 | 1-[2-(4-{[(3,4- dibromophenyl) methyl]amino}-1- piperidinyl)ethyl]- 1,5-naphthyridin- 2(1H)-one |
| 53 | 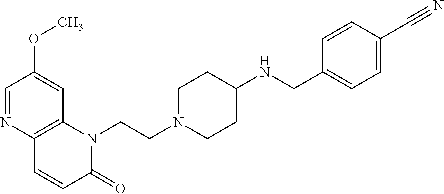 | 4-{[(1-{2-[7- (methyloxy)-2- oxo-1,5- naphthyridin- 1(2H)-yl]ethyl}-4- piperidinyl)amino] methyl}benzonitrile |
| 54 | 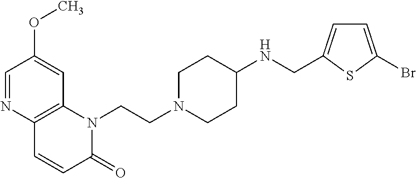 | 1-[2-(4-{[(5- bromo-2- thienyl)methyl] amino}-1- piperidinyl)ethyl]- 7-(methyloxy)- 1,5-naphthyridin- 2(1H)-one |
| 55 | 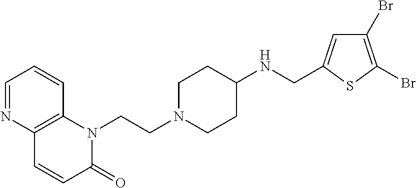 | 1-[2-(4-{[(4,5- dibromo-2- thienyl)methyl] amino}-1- piperidinyl)ethyl]- 1,5-naphthyridin- 2(1H)-one |
| 56 | 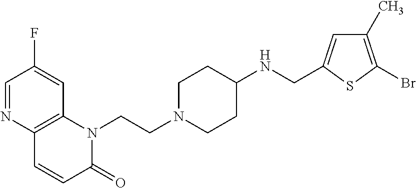 | 1-[2-(4-{[(5- bromo-4-methyl- 2-thienyl)methyl] amino}-1- piperidinyl)ethyl]- 7-fluoro-1,5- naphthyridin- 2(1H)-one |
| 57 | 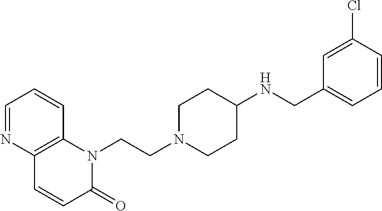 | 1-[2-(4-{[(3- chlorophenyl) methyl]amino}-1- piperidinyl)ethyl]- 1,5-naphthyridin- 2(1H)-one |
| 58 | 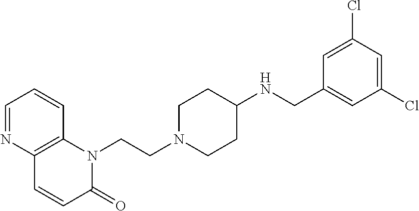 | 1-[2-(4-{[(3,5- dichlorophenyl) methyl]amino}-1- piperidinyl)ethyl]- 1,5-naphthyridin- 2(1H)-one |
| 59 | 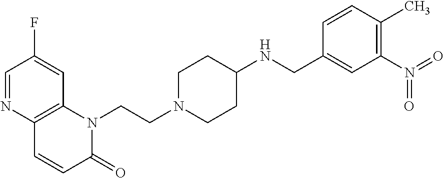 | 7-fluoro-1-[2-(4- {[(4-methyl-3- nitrophenyl)methyl] amino}-1- piperidinyl)ethyl]- 1,5-naphthyridin- 2(1H)-one |
| 60 | 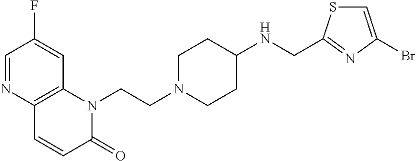 | 1-[2-(4-{[(4- bromo-1,3- thiazol-2- yl)methyl]amino}- 1-piperidinyl)ethyl]- 7-fluoro-1,5- naphthyridin- 2(1H)-one |
| 61 | 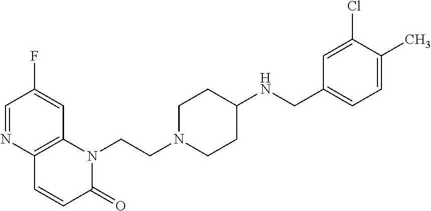 | 1-[2-(4-{[(3- chloro-4- methylphenyl) methyl]amino}-1- piperidinyl)ethyl]- 7-fluoro-1,5- naphthyridin- 2(1H)-one |
| 62 | 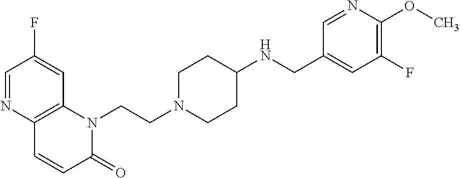 | 7-fluoro-1-{2-[4- ({[5-fluoro-6- (methyloxy)-3- pyridinyl]methyl} amino)-1- piperidinyl]ethyl}- 1,5-naphthyridin- 2(1H)-one |
| 63 | 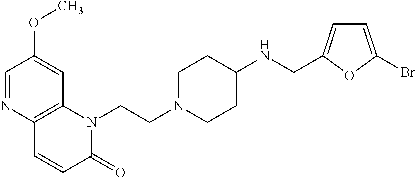 | 1-[2-(4-{[(5- bromo-2- furanyl)methyl] amino}-1- piperidinyl)ethyl]- 7-(methyloxy)- 1,5-naphthyridin- 2(1H)-one |
| 64 | 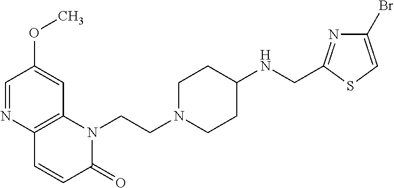 | 1-[2-(4-{[(4- bromo-1,3- thiazol-2- yl)methyl]amino}- 1-piperidinyl)ethyl]- 7-(methyloxy)- 1,5-naphthyridin- 2(1H)-one |
| 65 | 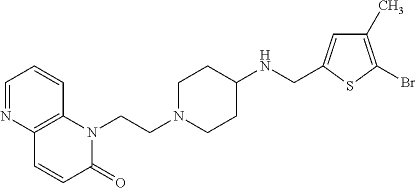 | 1-[2-(4-{[(5- bromo-4-methyl- 2-thienyl)methyl] amino}-1- piperidinyl)ethyl]- 1,5-naphthyridin- 2(1H)-one |
| 66 | 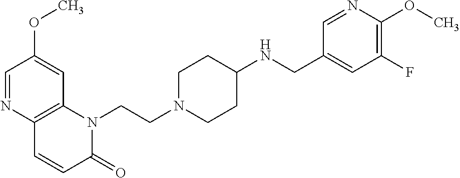 | 1-{2-[4-({[5- fluoro-6- (methloxy)-3- pyridinyl]methyl} amino)-1- piperidinyl]ethyl}- 7-(methyloxy)- 1,5-naphthyridin- 2(1H)-one |
| 67 | 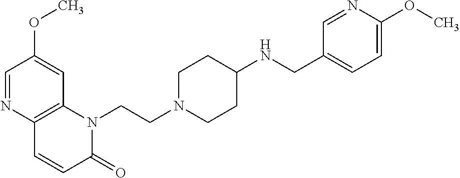 | 7-(methyloxy)-1- {2-[4-({[6- (methyloxy)-3- pyridinyl]methyl} amino)-1- piperidinyl]ethyl}- 1,5-naphthyridin- 2(1H)-one |
| 68 | 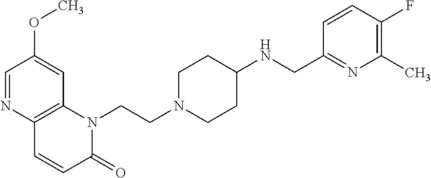 | 1-[2-(4-{[(5- fluoro-6-methyl- 2-pyridinyl)methyl] amino}-1- piperidinyl)ethyl]- 7-(methyloxy)- 1,5-naphthyridin- 2(1H)-one |
| 69 | 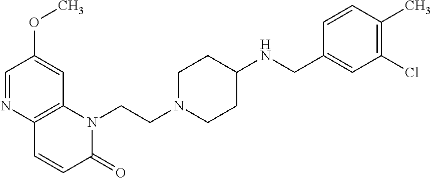 | 1-[2-(4-{[(3- chloro-4- methylphenyl) methyl]amino}-1- piperidinyl)ethyl]- 7-(methyloxy)- 1,5-naphthyridin- 2(1H)-one |
| 70 | 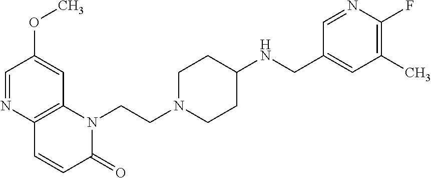 | 1-[2-(4-{[(6- fluoro-5-methyl- 3-pyridinyl)methyl] amino}-1- piperidinyl)ethyl]- 7-(methyloxy)- 1,5-naphthyridin- 2(1H)-one |
| 71 | 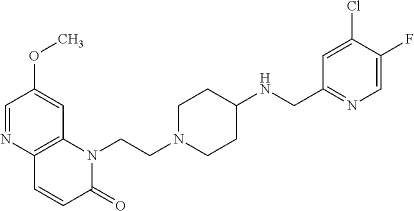 | 1-[2-(4-{[(4- chloro-5-fluoro-2- pyridinyl)methyl] amino}-1- piperidinyl)ethyl]- 7-(methyloxy)- 1,5-naphthyridin- 2(1H)-one |
| 72 | 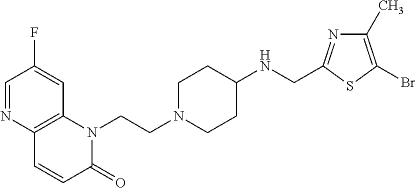 | 1-[2-(4-{[(5- bromo-4-methyl- 1,3-thiazol-2- yl)methyl]amino}-1- piperidinyl)ethyl]- 7-fluoro-1,5- naphthyridin- 2(1H)-one |
| 73 | 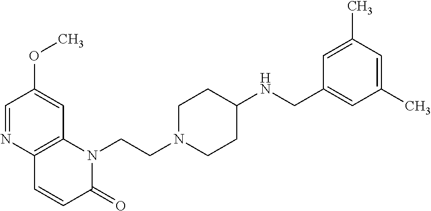 | 1-[2-(4-{[(3,5- Dimethylphenyl) methyl]amino}-1- piperidinyl)ethyl]- 7-(methyloxy)- 1,5-naphthyridin- 2(1H)-one |
| 74 | 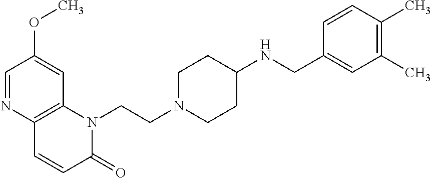 | 1-[2-(4-{[(3,4- Dimethylphenyl) methyl]amino}-1- piperidinyl)ethyl]- 7-(methyloxy)- 1,5-naphthyridin- 2(1H)-one |
| 75 | 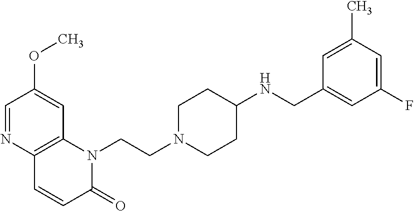 | 1-[2-(4-{[(3- fluoro-5- methylphenyl) methyl]amino}-1- piperidinyl)ethyl]- 7-(methyloxy)- 1,5-naphthyridin- 2(1H)-one |
| 76 | 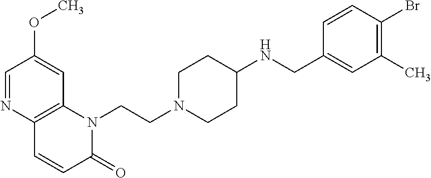 | 1-[2-(4-{[(4- bromo-3- methylphenyl) methyl]amino}-1- piperidinyl)ethyl]- 7-(methyloxy)- 1,5-naphthyridin- 2(1H)-one |
| 77 | 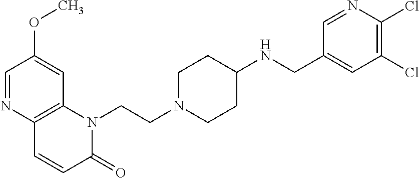 | 1-[2-(4-{[(5,6- dichloro-3- pyridinyl)methyl] amino}-1- piperidinyl)ethyl]- 7-(methyloxy)- 1,5-naphthyridin- 2(1H)-one |
| 78 | 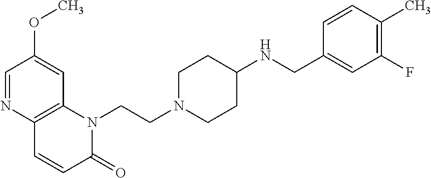 | 1-[2-(4-{[(3- fluoro-4- methylphenyl) methyl]amino}-1- piperidinyl)ethyl]- 7-(methyloxy)- 1,5-naphthyridin- 2(1H)-one |
| 79 | 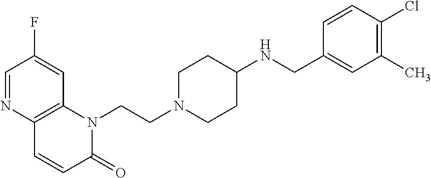 | 1-[2-(4-{[(4- chloro-3- methylphenyl) methyl]amino}-1- piperidinyl)ethyl]- 7-fluoro-1,5- naphthyridin- 2(1H)-one |
| 80 | 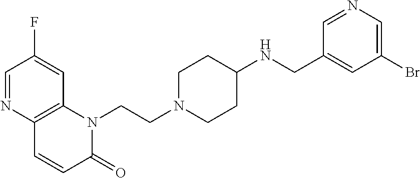 | 1-[2-(4-{[(5- bromo-3- pyridinyl)methyl] amino}-1- piperidinyl)ethyl]- 7-fluoro-1,5- naphthyridin- 2(1H)-one |
| 81 | 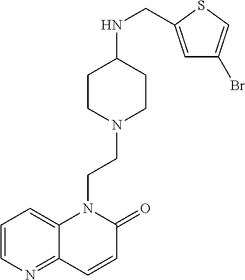 | 1-[2-(4-{[(4- bromo-2- thienyl)methyl] amino}-1- piperidinyl)ethyl]- 1,5-naphthyridin- 2(1H)-one |
| 82 | 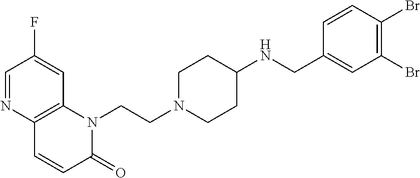 | 1-[2-(4-{[(3,4- dibromophenyl) methyl]amino}-1- piperidinyl)ethyl]- 7-fluoro-1,5- naphthyridin- 2(1H)-one |
| 83 | 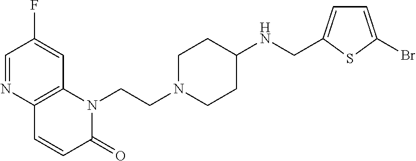 | 1-[2-(4-{[(5- bromo-2- thienyl)methyl] amino}-1- piperidinyl)ethyl]- 7-fluoro-1,5- naphthyridin- 2(1H)-one |
| 84 | 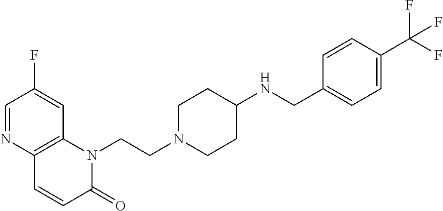 | 7-fluoro-1-{2-[4- ({[4-(trifluoromethyl) phenyl]methyl} amino)-1- piperidinyl]ethyl}- 1,5-naphthyridin- 2(1H)-one |
| 85 | 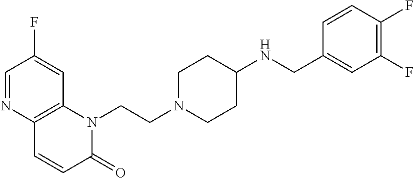 | 1-[2-(4-{[(3,4- difluorophenyl) methyl]amino}-1- piperidinyl)ethyl]- 7-fluoro-1,5- naphthyridin- 2(1H)-one |
| 86 | 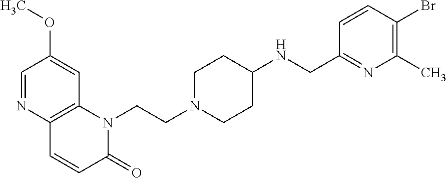 | 1-[2-(4-{[(5- bromo-6-methyl- 2-pyridinyl)methyl] amino}-1- piperidinyl)ethyl]- 7-(methyloxy)- 1,5-naphthyridin- 2(1H)-one |
| 87 | 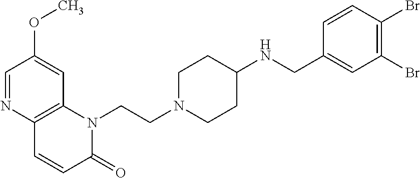 | 1-[2-(4-{[(3,4- dibromophenyl) methyl]amino}-1- piperidinyl)ethyl]- 7-(methyloxy)- 1,5-naphthyridin- 2(1H)-one |
| 88 | 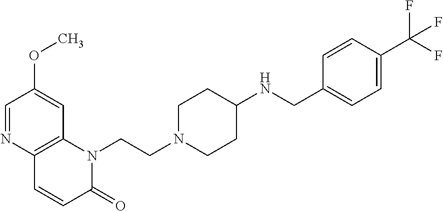 | 7-(methyloxy)-1- {2-[4-({[4- (trifluoromethyl) phenyl]methyl} amino)-1- piperidinyl]ethyl}- 1,5-naphthyridin- 2(1H)-one |
| 89 | 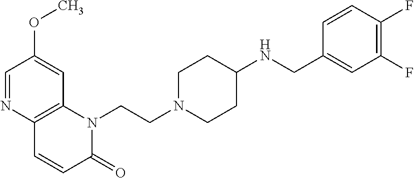 | 1-[2-(4-{[(3,4- difluorophenyl) methyl]amino}-1- piperidinyl)ethyl]- 7-(methyloxy)- 1,5-naphthyridin- 2(1H)-one |
| 90 | 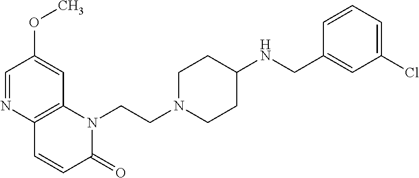 | 1-[2-(4-{[(3- chlorophenyl) methyl]amino}-1- piperidinyl)ethyl]- 7-(methyloxy)- 1,5-naphthyridin- 2(1H)-one |
| 91 | 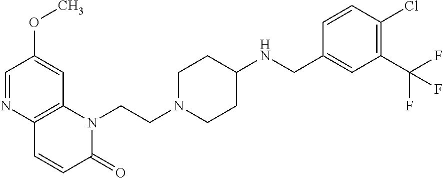 | 1-{2-[4-({[4- chloro-3- (trifluoromethyl) phenyl]methyl} amino)-1- piperidinyl]ethyl}- 7-(methyloxy)- 1,5-naphthyridin- 2(1H)-one |
| 92 | 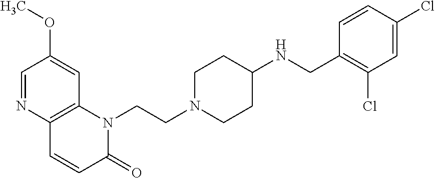 | 1-[2-(4-{[(2,4- dichlorophenyl) methyl]amino}-1- piperidinyl)ethyl]- 7-(methyloxy)- 1,5-naphthyridin- 2(1H)-one |
| 93 | 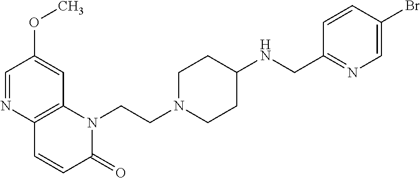 | 1-[2-(4-{[(5- bromo-2- pyridinyl)methyl] amino}-1- piperidinyl)ethyl]- 7-(methyloxy)- 1,5-naphthyridin- 2(1H)-one |
| 94 | 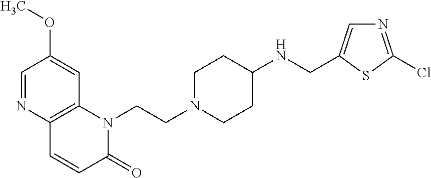 | 1-[2-(4-{[(2- chloro-1,3- thiazol-5- yl)methyl]amino}- 1-piperidinyl)ethyl]- 7-(methyloxy)- 1,5-naphthyridin- 2(1H)-one |
| 95 | 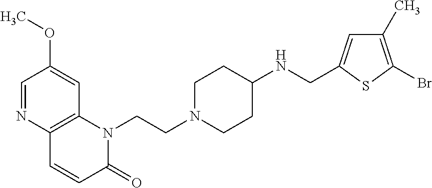 | 1-[2-(4-{[(5- bromo-4-methyl- 2-thienyl)methyl] amino}-1- piperidinyl)ethyl]- 7-(methyloxy)- 1,5-naphthyridin- 2(1H)-one |
| 96 | 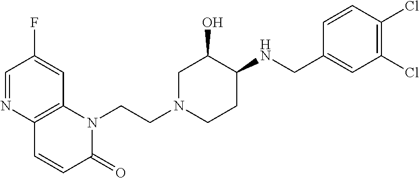 | 1-[2-((3R,4S)-4- {[(3,4-dichlorophenyl) methyl]amino}-3- hydroxy-1- piperidinyl)ethyl]- 7-fluoro-1,5- naphthyridin- 2(1H)-one |
| 97 | 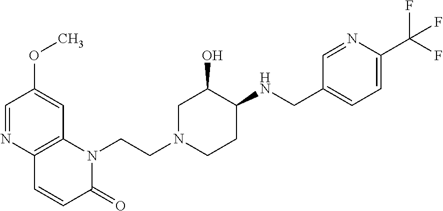 | 1-{2-[(3R,4S)-3- hydroxy-4-({[6- (trifluoromethyl)- 3-pyridinyl]methyl} amino)-1- piperidinyl]ethyl}- 7-(methyloxy)- 1,5-naphthyridin- 2(1H)-one |
| 98 | 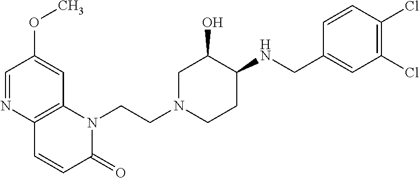 | 1-[2-((3R,4S)-4- {[(3,4-dichlorophenyl) methyl]amino}-3- hydroxy-1- piperidinyl)ethyl]- 7-(methyloxy)- 1,5-naphthyridin- 2(1H)-one |
| 99 | 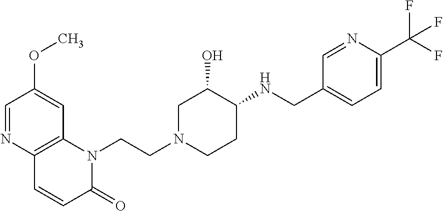 | 1-{2-[(3S,4R)-3- hydroxy-4-({[6- (trifluoromethyl)- 3-pyridinyl]methyl} amino)-1- piperidinyl]ethyl}- 7-(methyloxy)- 1,5-naphthyridin- 2(1H)-one |
| 100 | 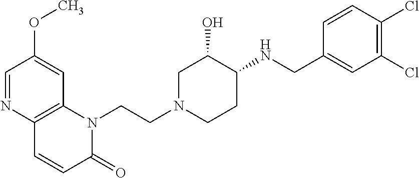 | 1-[2-((3S,4R)-4- {[(3,4-dichlorophenyl) methyl]amino}-3- hydroxy-1- piperidinyl)ethyl]- 7-(methyloxy)- 1,5-napthyridin- 2(1H)-one |
| 101 | 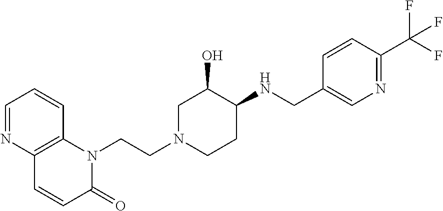 | 1-{2-[(3R,4S)-3- hydroxy-4-({[6- (trifluoromethyl)- 3-pyridinyl]methyl} amino)-1- piperidinyl]ethyl}- 1,5-naphthyridin- 2(1H)-one |
| 102 | 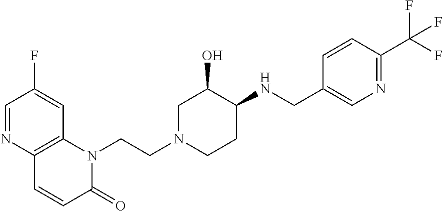 | 7-fluoro-1-{2- [(3R,4S)-3- hydroxy-4-({[6- (trifluoromethyl)- 3-pyridinyl]methyl} amino)-1- piperidinyl]ethyl}- 1,5-naphthyridin- 2(1H)-one |
| 103 | 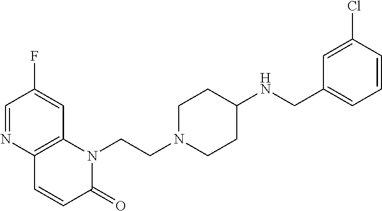 | 1-[2-(4-{[(3- chlorophenyl) methyl]amino}-1- piperidinyl)ethyl]- 7-fluoro-1,5- naphthyridin- 2(1H)-one |
| 104 | 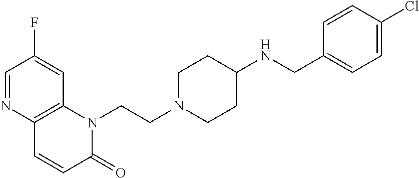 | 1-[2-(4-{[(4- chlorophenyl) methyl]amino}-1- piperidinyl)ethyl]- 7-fluoro-1,5- naphthyridin- 2(1H)-one |
| 105 | 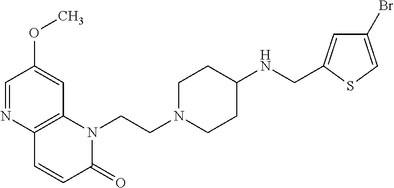 | 1-[2-(4-{[(4- bromo-2- thienyl)methyl] amino}-1- piperidinyl)ethyl]- 7-(methyloxy)- 1,5-naphthyridin- 2(1H)-one |
| 106 | 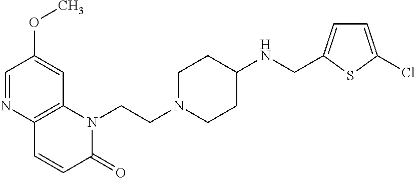 | 1-[2-(4-{[(5- chloro-2- thienyl)methyl] amino}-1- piperidinyl)ethyl]- 7-(methyloxy)- 1,5-naphthyridin- 2(1H)-one |
| 107 | 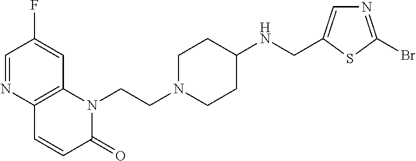 | 1-[2-(4-{[(2- bromo-1,3- thiazol-5- yl)methyl]amino}- 1-piperidinyl)ethyl]- 7-fluoro-1,5- naphthyridin- 2(1H)-one |
| 108 | 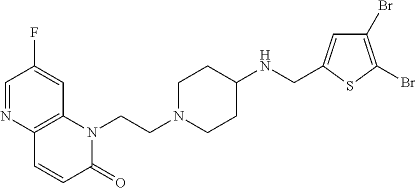 | 1-[2-(4-{[(4,5- dibromo-2- thienyl)methyl] amino}-1- piperidinyl)ethyl]- 7-fluoro-1,5- naphthyridin- 2(1H)-one |
| 109 | 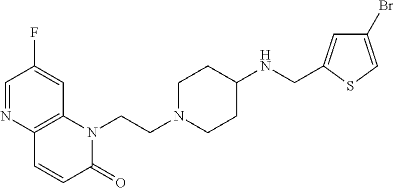 | 1-[2-(4-{[(4- bromo-2- thienyl)methyl] amino}-1- piperidinyl)ethyl]- 7-fluoro-1,5- naphthyridin- 2(1H)-one |
| 110 | 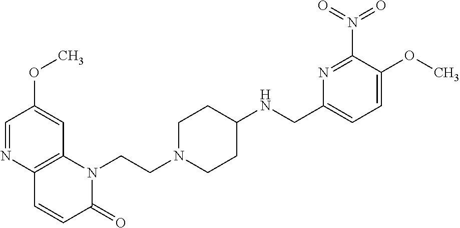 | 7-(methyloxy)-1- {2-[4-({[5- (methyloxy)-6- nitro-2- pyridinyl]methyl} amino)-1- piperidinyl]ethyl}- 1,5-naphthyridin- 2(1H)-one |
| 111 | 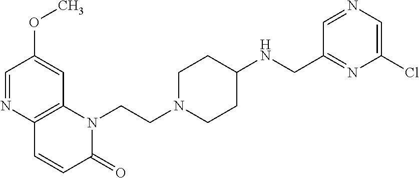 | 1-[2-(4-{[(6- chloro-2- pyrazinyl)methyl]- amino}-1- piperidinyl)ethyl]- 7-(methyloxy)- 1,5-naphthyridin- 2(1H)-one |
| 112 | 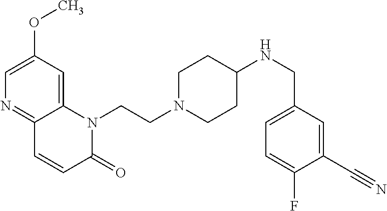 | 2-fluoro-5-{[(1- {2-[7-(methyloxy)- 2-oxo-1,5- naphthyridin- 1(2H)-yl]ethyl}- 4-piperidinyl) amino]methyl} benzonitrile |
| 113 | 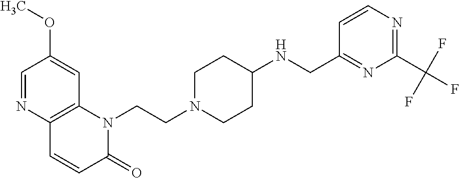 | 7-(methyloxy)- 1-{2-[4-({[2- (trifluoromethyl)- 4-pyrimidinyl]methyl} amino)-1- piperidinyl]ethyl}- 1,5-naphthyridin- 2(1H)-one |
| 114 | 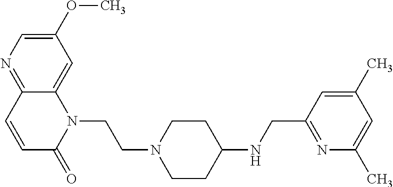 | 1-[2-(4-{[(4,6- dimethyl-2- pyridinyl)methyl] amino}-1-piperidinyl) ethyl]-7- (methyloxy)-1,5- naphthyridin- 2(1H)-one |
| 115 | 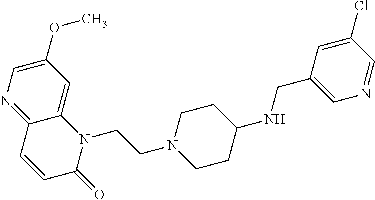 | 1-[2-(4-{[(5- chloro-3- pyridinyl)methyl] amino}-1- piperidinyl)ethyl]- 7-(methyloxy)- 1,5-naphthyridin- 2(1H)-one |
| 116 | 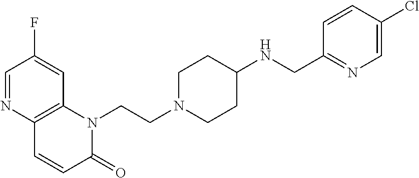 | 1-[2-(4-{[(5- chloro-2- pyridinyl)methyl] amino}-1- piperidinyl)ethyl]- 7-(methyloxy)- 1,5-naphthyridin- 2(1H)-one |
| 117 | 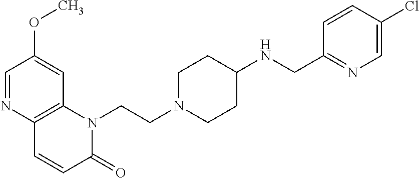 | 1-[2-((3R,4S)- 4-{[(5-chloro-6- methyl-3- pyridinyl)methyl] amino}-3- hydroxy-1- piperidinyl)ethyl]- 7-(methyloxy)- 1,5-naphthyridin- 2(1H)-one |
| 118 | 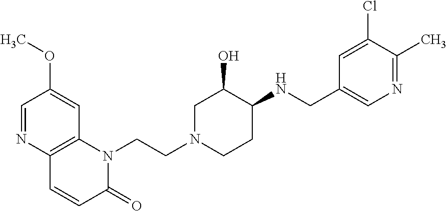 | 1-[2-((3R,4S)- 4-{[(5-chloro-6- methyl-3- pyridinyl)methyl] amino}-3- hydroxy-1- piperidinyl)ethyl]- 7-(methyloxy)- 1,5-naphthyridin- 2(1H)-one |
| 119 | 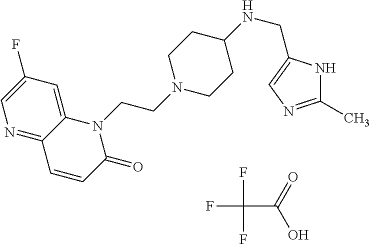 | 7-fluoro-1-[2-(4- {[(2-methyl-1H- imidazol-4- yl)methyl]amino}- 1-piperidinyl)ethyl]- 1,5-naphthyridin- 2(1H)-one trifluoroacetate |
| 120 | 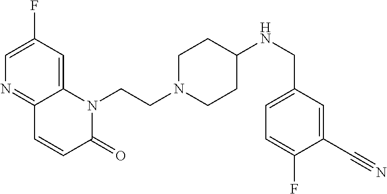 | 2-fluoro-5-[({1- [2-(7-fluoro-2- oxo-1,5- naphthyridin- 1(2H)-yl)ethyl]- 4-piperidinyl}amino) methyl]benzonitrile |
| 121 | 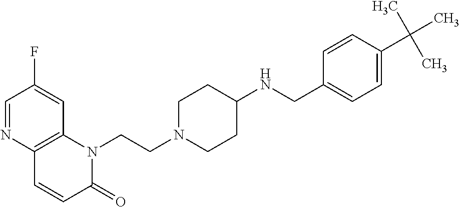 | 1-{2-[4-({[4- (1,1- dimethylethyl) phenyl]methyl} amino)-1- piperidinyl]ethyl}- 7-fluoro-1,5- naphthyridin- 2(1H)-one |
| 122 | 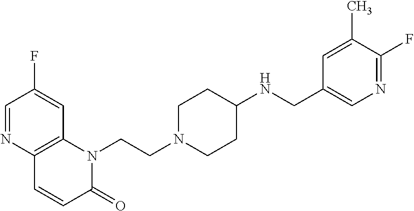 | 7-fluoro-1-[2-(4- {[(6-fluoro-5- methyl-3- pyridinyl)methyl] amino}-1- piperidinyl)ethyl]- 1,5-naphthyridin- 2(1H)-one |
| 123 | 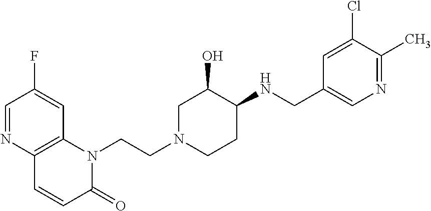 | 1-[2-((3R,4S)- 4-{[(5-chloro-6- methyl-3- pyridinyl)methyl] amino}-3- hydroxy-1- piperidinyl)ethyl]- 7-fluoro-1,5- naphthyridin- 2(1H)-one hydrochloride |
| 124 | 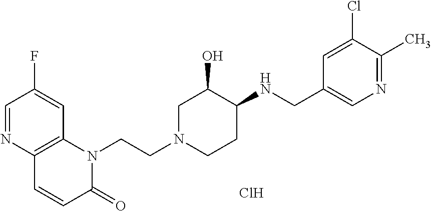 | 1-[2-((3R,4S)- 4-{[(5-chloro-6- methyl-3- pyridinyl)methyl] amino}-3- hydroxy-1- piperidinyl)ethyl]- 7-fluoro-1,5- naphthyridin- 2(1H)-one hydrochloride |
| 125 | 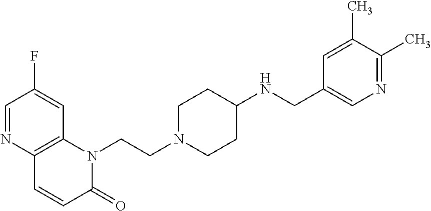 | 1-[2-(4-{[(5,6- dimethyl-3- pyridinyl)methyl] amino}-1- piperidinyl)ethyl]- 7-fluoro-1,5- naphthyridin- 2(1H)-one |
| 126 | 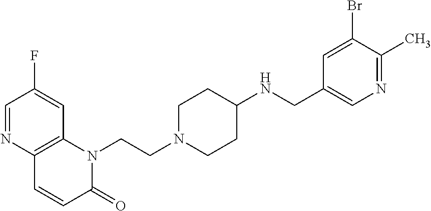 | 1-[2-(4-{[(5- bromo-6- methyl-3- pyridinyl)methyl] amino}-1- piperidinyl)ethyl]- 7-fluoro-1,5- naphthyridin- 2(1H)-one |
| 127 | 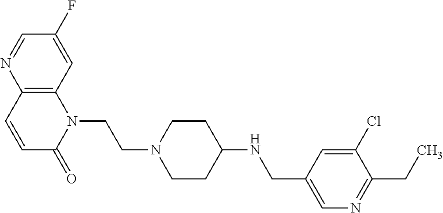 | 1-[2-(4-{[(5- chloro-6-ethyl- 3-pyridinyl) methyl]amino}- 1-piperidinyl) ethyl]-7-fluoro- 1,5- naphthyridin- 2(1H)-one |
| 128 | 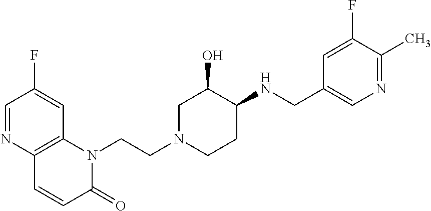 | 7-fluoro-1-[2- ((3R,4S)-4-{[(5- fluoro-6-methyl- 3-pyridinyl)methyl] amino}-3- hydroxy-1- piperidinyl)ethyl]- 1,5-naphthyridin- 2(1H)-one |
| 129 | 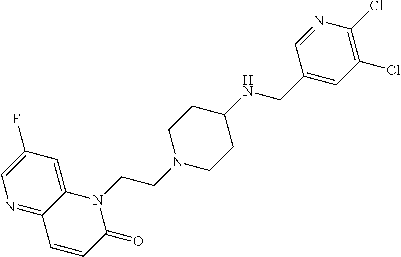 | 1-[2-(4-{[(5,6- dichloro-3- pyridinyl)methyl] amino}-1- piperidinyl)ethyl]- 7-fluoro-1,5- naphthyridin- 2(1H)-one |
| 130 | 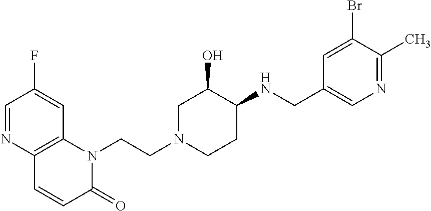 | 1-[2-((3R,4S)- 4-{[(5-bromo-6- methyl-3- pyridinyl) methyl]amino}- 3-hydroxy-1- piperidinyl) ethyl]-7-fluoro- 1,5-naphthyridin- 2(1H)-one |
| 131 | 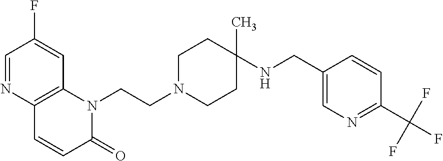 | 7-fluoro-1-{2-[4- methyl-4-({[6- (trifluoromethyl)- 3-pyridinyl] methyl}amino)- 1-piperidinyl] ethyl}-1,5- naphthyridin- 2(1H)-one |
| 132 | 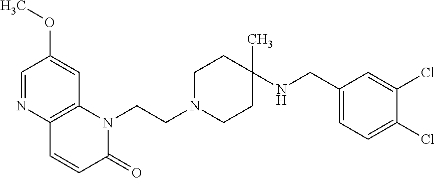 | 1-[2-(4-{[(3,4- dichlorophenyl) methyl]amino}- 4-methyl-1- piperidinyl)ethyl]- 7-(methyloxy)- 1,5-naphthyridin- 2(1H)-one |
| 133 | 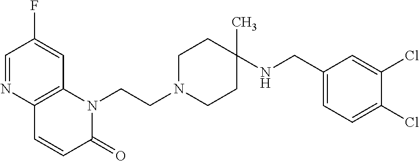 | 1-[2-(4-{[(3,4- dichlorophenyl) methyl]amino}- 4-methyl-1- piperidinyl)ethyl]-7- fluoro-1,5- naphthyridin- 2(1H)-one |
| 134 | 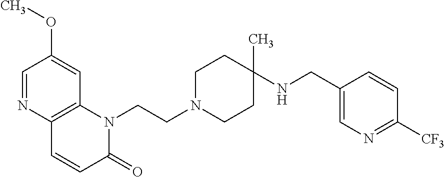 | 7-(methyloxy)- 1-{2-[4-methyl- 4-({[6-(trifluoro methyl)-3- pyridinyl]methyl} amino)-1- piperidinyl] ethyl}-1,5- naphthyridin- 2(1H)-one |

| | | Method | Starting | |
|---|---|---|---|---|
| | | analogous to | aldehyde* | Starting |
| Ex. | Physical data | which Ex. | (source) | Naphthyridone |
| 17 | ¹H-NMR (δ, ppm, DMSO-d₆): 8.25 (d, | Example 1 | 4- | Intermediate 38 |
| | 1H); 7.84 (d, 1 H); 7.38-7.37 (m, 1 H); | | chlorobenzaldehyde | |
| | 7.34 (s, 4 H); 6.64 (d, 1 H); 4.32-4.22 | | (Fluka) | |
| | (m, 4 H); 3.69 (s, 2 H); 2.91-2.87 (m, | | | |
| | 2 H); 2.38-2.30 (m, 1 H); 2.03-1.95 (m, | | | |
| | 2H); 1.78-1.73 (m, 2 H); 1.39 (t, 3 H); | | | |
| | 1.27-1.16 (m, 2 H). [ES MS] m/z 441 | | | |
| | (MH+). | | | |
| 18 | ¹H-NMR (δ, ppm, DMSO-d₆): 8.69 (s, | Example 1 | 6- | Intermediate 38 |
| | 1 H); 8.25 (d, 1 H); 8.02 (d, 1 H); 7.86- | | (trifluoromethyl) | |
| | 7.82 (m, 2 H); 7.38-7.37 (m, 1 H); 6.63 | | pyridine-3- | |
| | (d, 1 H); 4.35-4.22 (m, 4 H); 3.82 (s, | | carboxaldehyde | |
| | 2 H); 2.91-2.88 (m, 2 H); 2.37-2.29 (m, | | (Appollo) | |
| | 1 H); 2.04-1.97 (m, 2 H); 1.79-1.75 (m, | | | |
| | 2 H); 1.39 (t, 3 H); 1.26-1.15 (m, 2 H). | | | |
| | [ES MS] m/z 476 (MH+). | | | |
| 19 | ¹H-NMR (δ, ppm, DMSO-d₆): 8.25 | Example 1 | 1-[2-(4-{[(3,4- | Intermediate 38 |
| | (d, 1 H); 7.84 (d, 1 H); 7.59 (d, 1 H); | | dichlorophenyl) | |
| | 7.53 (d, 1 H); 7.38 (d, 1 H); 7.33-7.29 | | methyl]amino}-1- | |
| | (m, 1 H); 6.63 (d, 1 H); 4.34-4.22 (m, | | piperidinyl)ethyl]- | |
| | 4 H); 3.69 (s, 2 H); 2.90-2.87 (m, 2 H); | | 7-(ethyloxy)-1,5- | |
| | 2.35-2.25 (m, 1 H); 2.03-1.96 (m, 2 H); | | naphthyridin- | |
| | 1.76-1.72 (m, 2 H); 1.39 (t, 3 H); 1.25- | | 2(1H)-one | |
| | 1.14 (m, 2 H). [ES MS] m/z 475 | | (Fluka) | |
| | (MH+). | | | |
| 20 | ¹H-NMR (δ, ppm, CDCl₃): 8.36 (d, 1 H); | Example 1 | 5-chloro-6- | Intermediate 21 |
| | 8.28 (d, 1 H); 7.85 (d, 1 H); 7.54 (bd, | | methyl-3- | |
| | 1 H); 7.34 (dd, 1 H); 6.74 (d, 1 H); 4.39 | | pyridinecarbaldehyde | |
| | (bd, 2 H); 3.99 (s, 3 H); 3.92 (s, 2 H); | | (Described in | |
| | 3.08-2.96 (m, 2 H); 2.72-2.62 (m, 2 H); | | WO20006/137485 A1) | |
| | 2.62-2.51 (m, 1 H); 2.32-2.14 (m, 2 H); | | | |
| | 1.99-1.80 (m, 2 H); 1.76-1.59 (m, 3 H). | | | |
| | [ES MS] m/z 472 (MH+). | | | |
| 21 | ¹H-NMR (δ, ppm, CDCl₃): 8.56 (d, | Example 1 | 4-bromo-2- | Intermediate 29 |
| | 1 H); 8.46 (d, 1 H); 8.28 (d, 1 H); 8.88- | | pyridinecarbaldehyde | |
| | 8.83 (m, 2 H); 7.22 (bd, 1 H); 4.37 (t, | | (PRINCETON) | |
| | 2 H); 3.98 (s, 3 H); 3.82 (s, 2 H); 2.98 | | | |
| | (bd, 2 H); 2.65 (bt, 2 H); 2.19 (bt, 2 H); | | | |
| | 2.23 (bt, 2 H); 1.87-1.99 (m, 2 H); 1.60- | | | |
| | 1.37 (m, 4 H). [ES MS] m/z 472 (MH+). | | | |
| 22 | ¹H-NMR (δ, ppm, CDCl₃): 8.27 (d, | Example 1 | 5-bromo-3- | Intermediate 29 |
| | 1 H); 7.84 (d, 1 H); 7.29-7.23 (m, 2 H); | | pyridinecarbaldehyde | |
| | 7.18 (bs, 1 H); 7.06 (dd, 1 H); 7.74 (d, | | (Aldrich) | |
| | 1 H); 4.37 (bt, 2 H); 3.97 (s, 3 H); 3.75 | | | |
| | (s, 2 H); 2.99 (bd, 2 H); 2.65 (bt, 2 H); | | | |
| | 2.58-2.45 (m, 1 H); 2.36 (s, 3 H); 2.19 | | | |
| | (bt, 2 H); 1.91 (bd, 2 H). [ES MS] m/z | | | |
| | 441 (MH+). | | | |
| 23 | ¹H-NMR (δ, ppm, CDCl₃): 8.55 (d, | Example 1 | 5-chloro-6- | Intermediate 29 |
| | 1 H); 7.91 (d, 1 H); 7.81 (d, 1 H); 7.49- | | methyl-3- | |
| | 7.44 (m, 1 H); 7.29 (d, 1 H); 7.16 (d, | | pyridinecarbaldehyde | |
| | 1 H); 7.09 (d, 1 H); 6.91 (d, 1 H); 4.40 | | (Described in | |
| | (t, 2 H); 3.75 (s, 2 H); 2.99 (d, 2 H); | | WO20006/137485 A1) | |
| | 2.65 (t, 2 H); 2.57-2.48 (m, 1 H); 2.35 | | | |
| | (s, 3 H); 2.20 (t, 2 H); 1.90 (d, 2 H); | | | |
| | 1.50-1.36 (m, 2 H). [ES MS] m/z 411 | | | |
| | (MH+). | | | |
| 24 | ¹H-NMR (δ, ppm, CDCl₃): 8.56 (d, | Example 1 | 4-chloro-3- | Intermediate 29 |
| | 1 H); 7.93 (d, 1 H); 7.72 (d, 1 H); 7.48 (t, | | methylbenzaldehyde | |
| | 1 H); 7.46-7.44 (m, 1 H); 7.38 (d, 1 H); | | (Fluorochem) | |
| | 7.29-7.24 (m, 1 H); 7.18 (dd, 1 H); | | | |
| | 4.55-4.24 (m, 2 H); 3.90-3.84 (m, 2 H); | | | |
| | 3.78 (d, 2 H); 2.98 (dd, 2 H); 2.70 (bt, | | | |
| | 2 H); 2.51-2.42 (m, 1 H). [ES MS] m/z | | | |
| | 447 (MH+). | | | |
| 25 | ¹H-NMR (δ, ppm, CDCl₃): 8.41 (d, 1 H); | Example 1 | 3-Chloro-4- | Intermediate 8 |
| | 7.88 (d, 1 H); 7.54 (d, 1 H); 7.08 (d, | | methylbenzaldehyde | |
| | 2 H); 7.03 (d, 1 H); 6.85 (d, 1 H); 4.31 | | (Aldrich) | |
| | (bt, 2 H); 3.74 (s, 2 H); 2.94 (bd, 2 H); | | | |
| | 2.64 (bt, 2 H); 2.59-2.47 (m, 1 H); 2.25 | | | |
| | (s, 3 H); 2.24 (s, 3 H); 2.16 (bd, 2 H); | | | |
| | 1.91 (bd, 2 H); 1.50-1.34 (bd, 3 H). [ES | | | |
| | MS] m/z 409 (MH+). | | | |
| 26 | ¹H-NMR (δ, ppm, CDCl₃): 8.54 (d, | Example 1 | 3,4- | Intermediate 35b |
| | 1 H); 7.92 (d, 1 H); 7.80 (bd, 1 H); 7.49- | | dichlorobenzaldehyde | |
| | 7.44 (m, 1 H); 7.28 (d, 1 H); 7.18 (bs, | | (Fluka) | |
| | 1 H); 7.07 (bd, 1 H); 6.91 (d, 1 H); 4.40 | | | |
| | (t, 2 H); 3.74 (s, 2 H); 2.99 (d, 2 H); | | | |
| | 2.65 (t, 2 H); 2.58-2.48 (m, 1 H); 2.36 | | | |
| | (s, 3 H); 2.20 (bt, 2 H); 1.90 (d, 2 H); | | | |
| | 1.52-1.35 (m, 2 H). [ES MS] m/z 411 | | | |
| | (MH+). | | | |
| 27 | ¹H-NMR (δ, ppm, CDCl₃): 8.55 (d, | Example 1 | 3,4- | Intermediate 21 |
| | 1 H); 7.92 (d, 1 H); 7.83 (bd, 1 H); 7.49- | | Dimethylbenzaldehyde | |
| | 7.45 (m, 1 H); 7.12 (t, 1 H); 7.00-6.96 | | (Aldrich) | |
| | (m, 1 H); 6.91 (d, 1 H); 4.41 (t, 2 H); | | | |
| | 3.77 (s, 2 H); 3.00 (bd, 2 H); 2.66 (t, | | | |
| | 2 H); 2.61-2.49 (m, 1 H); 2.30-2.16 (m, | | | |
| | 5 H); 2.02-1.85 (m, 7 H); 1.53-1.38 (m, | | | |
| | 2 H). [ES MS] m/z 395 (MH+). | | | |
| 28 | ¹H-NMR (δ, ppm, CDCl₃): 8.55 (d, | Example 1 | 4-Chloro-3- | Intermediate 8 |
| | 1 H); 7.90 (d, 1 H); 7.81 (bd, 1 H); 7.50- | | methylbenzaldehyde | |
| | 7.44 (m, 1 H); 7.13-7.01 (m, 3 H); | | (′Fluorochem) | |
| | 6.91 (d, 1 H); 4.39 (t, 2 H); 3.75 (s, 2 H); | | | |
| | 2.97 (bd, 2 H); 2.66 (t, 2 H); 2.61-2.49 | | | |
| | (m, 1 H); 2.31-2.13 (m, 8 H); 2.02-1.85 | | | |
| | (m, 2 H); 1.53-1.38 (m, 2 H). [ES MS] | | | |
| | m/z 391 (MH+). | | | |
| 29 | ¹H-NMR (δ, ppm, CDCl₃): 8.41 (d, 1 H); | Example 1 | 5-chloro-6- | Intermediate 8 |
| | 7.88 (d, 1 H); 7.55 (bd, 1 H); 6.86 (d, | | methyl-3- | |
| | 1 H); 6.21 (d, 1 H); 6.14 (d, 1 H); 4.32 | | pyridinecarbaldehyde | |
| | (bt, 2 H); 3.77 (s, 2 H); 2.96 (bd, 2 H); | | (Described in | |
| | 2.65 (bt, 2 H); 2.58-2.44 (m, 1 H); | | WO20006/137485 A1) | |
| | 2.18 (bt, 2 H); 1.87 (bd, 2 H); 1.48- | | | |
| | 1.31 (m, 2 H). [ES MS] m/z 449 | | | |
| | (MH+). | | | |
| 30 | ¹H-NMR (δ, ppm, CDCl₃): 8.68 (s, 1 H); | Example 1 | 5-bromo-6- | Intermediate 21 |
| | 8.54 (dd, 1 H); 7.91 (d, 1 H); 7.89 (d, | | methyl-2- | |
| | 1 H); 7.78 (bd, 1 H); 7.64 (d, 1 H); 7.50- | | pyridinecarbaldehyde | |
| | 7.43 (m, 1 H); 6.92 (d, 1 H); 4.40 (bt, | | (Intermediate 52) | |
| | 2 H); 3.92 (s, 2 H); 2.99 (bd, 2 H); | | | |
| | 2.66 (bt, 2 H); 2.59-2.46 (m, 1 H); | | | |
| | 2.20 (bt, 2 H); 1.92 (bd, 2 H); 1.51- | | | |
| | 1.34 (m, 2 H). [ES MS] m/z 432 (MH+). | | | |
| 31 | ¹H-NMR (δ, ppm, CDCl₃): 8.54 (dd, | Example 1 | 3-Fluoro-4- | Intermediate 8 |
| | 1 H); 7.91 (d, 1 H); 7.79 (bd, 1 H); | | methylbenzaldehyde | |
| | 7.67 (s, 1 H); 7.48 (d, 1 H); 7.45 (s, 2 H); | | (Alfa Aesar) | |
| | 6.92 (d, 1 H); 4.40 (bt, 2 H); 3.83 (s, 2 | | | |
| | H); 2.99 (bd, 2 H); 2.65 (bt, 2 H); 2.58- | | | |
| | 2.45 (m, 1 H); 2.19 (bt, 2 H); 1.91 (bd, | | | |
| | 2 H); 1.51-1.33 (m, 2 H). [ES MS] m/z | | | |
| | 465. | | | |
| 32 | ¹H-NMR (δ, ppm, CDCl₃): 8.28 (d, 1 H); | Example 1 | 3,4- | Intermediate 8 |
| | 8.18 (d, 2 H); 7.84 (d, 1 H); 7.51 (d, 2 H); | | Dimethylbenzaldehyde | |
| | 7.21 (bs, 1 H); 6.74 (d, 1 H); 4.37 (bt, | | (Aldrich) | |
| | 2 H); 3.98 (s, 3 H); 3.93 (s, 2 H); | | | |
| | 3.00 (bd, 2 H); 2.65 (bt, 2 H); 2.60- | | | |
| | 2.46 (m, 1 H); 2.19 (bt, 2 H); 1.92 (bd, | | | |
| | 2 H); 1.48-1.35 (m, 2 H). [ES MS] m/z | | | |
| | 438 (MH+). | | | |
| 33 | ¹H-NMR (δ, ppm, CDCl₃): 8.41 (d, 1 H); | Example 1 | 4-chloro-5-fluoro- | Intermediate 21 |
| | 7.88 (d, 1 H); 7.54 (dd, 1 H); 7.44 (d, | | 2- | |
| | 1 H); 7.27 (d, 1 H); 7.17 (dd, 1 H); 6.86 | | pyridinecarbaldehyde | |
| | (d, 1 H); 4.32 (t, 2 H); 3.86 (s, 2 H); | | (Intermediate 48) | |
| | 2.96 (bs, 2 H); 2.65 (t, 2 H); 2.58-2.46 | | | |
| | (m, 1 H); 2.20 (t, 2 H); 1.92 (bd, 2 H); | | | |
| | 1.50-1.34 (m, 2 H). [ES MS] m/z 449 | | | |
| | (MH+). | | | |
| 34 | ¹H-NMR (δ, ppm, CDCl₃): 8.31 (d, | Example 1 | 5-bromo-2- | Intermediate 21 |
| | 1 H); 8.27 (d, 1 H); 7.85 (d, 1 H); 7.72- | | furancarbaldehyde | |
| | 7.69 (dd, 1 H); 7.56 (d, 1 H); 7.04 (d, | | (Aldrich) | |
| | 1 H); 6.65 (d, 1 H); 4.33 (t, 2 H); 3.96 | | | |
| | (s, 3 H); 3.68 (s, 2 H); 2.92-2.88 (m, | | | |
| | 2 H); 2.35-2.26 (m, 2 H); 2.05-1.97 (m, | | | |
| | 2 H); 1.77-1.73 (m, 2 H); 1.25-1.13 | | | |
| | (m, 2 H). [ES MS] m/z 472 (MH+). | | | |
| 35 | ¹H-NMR (δ, ppm, CDCl₃): 8.42 (d, 1 H); | Example 1 | 6- | Intermediate 8 |
| | 7.89 (d, 1 H); 7.56-7.53 (m, 1 H); 7.23 | | (trifluoromethyl)- | |
| | (s, 1 H); 6.86 (d, 1 H); 4.35-4.30 (m, | | 3- | |
| | 2 H); 3.77 (s, 2 H); 2.99-2.95 (m, 2 H); | | pyridinecarbaldehyde | |
| | 2.68-2.63 (m, 2 H); 2.55-2.46 (m, 1 H); | | (Apollo) | |
| | 2.23-2.15 (m, 2 H); 1.93-1.88 (m, 2 H); | | | |
| | 1.47-1.33 (m, 2 H). [ES MS] m/z 449 | | | |
| | (MH+). | | | |
| 36 | ¹H-NMR (δ, ppm, DMSO-d₆): 8.27 (d, | Example 1 | 4-chloro-3- | Intermediate 8 |
| | 1 H); 7.85 (d, 1 H); 7.40-7.32 (m, 3 H); | | (trifluoromethyl) | |
| | 7.12-7.06 (m, 2 H); 6.65 (d, 1 H); 4.33 | | benzaldehyde | |
| | (t, 2 H); 3.96 (s, 3 H); 3.67 (s, 2 H); | | (Aldrich) | |
| | 2.92-2.88 (m, 2 H); 2.37-2.28 (m, 1 H); | | | |
| | 2.04-1.97 (m, 2 H); 1.78-1.74 (m, 2 H); | | | |
| | 1.26-1.14 (m, 2 H). [ES MS] m/z 411 | | | |
| | (MH+). | | | |
| 37 | ¹H-NMR (δ, ppm, DMSO-d₆): 8.52- | Example 1 | 4- | Intermediate 29 |
| | 8.50 (m, 1 H); 8.00 (d, 1 H); 7.93 (d, | | nitrobenzaldehyde | |
| | 1 H); 7.66-7.53 (m, 5 H); 6.85 (d, 1 H); | | (Aldrich) | |
| | 4.31 (t, 2 H); 3.79 (s, 2 H); 2.90-2.86 | | | |
| | (m, 2 H); 2.36-2.30 (m, 1 H); 2.04-1.97 | | | |
| | (m, 2 H); 1.79-1.74 (m, 2 H); 1.28-1.18 | | | |
| | (m, 2 H); [ES MS] m/z 431. | | | |
| 38 | ¹H-NMR (δ, ppm, DMSO-d₆): 8.52- | Example 1 | 2,5- | Intermediate 21 |
| | 8.50 (m, 1 H); 8.00 (d, 1 H); 7.93 (d, | | dichlorobenzaldehyde | |
| | 1 H); 7.62-7.58 (m, 1 H); 7.34 (s, 4 H); | | (Alfa Aesar) | |
| | 6.85 (d, 1 H); 4.31 (t, 2 H); 3.70 (s, 2 H); | | | |
| | 2.90-2.86 (m, 2 H); 2.39-2.31 (m, 1 H); | | | |
| | 2.04-1.96 (m, 2 H); 1.77-1.74 (m, 2 H); | | | |
| | 1.26-1.16 (m, 2 H). [ES MS] m/z 397 | | | |
| | (MH+). | | | |
| 39 | 1H-NMR (δ, ppm, CDCl₃): 8.42 (d, | Example 1 | 6-bromo-3- | Intermediate 29 |
| | 1 H); 7.88 (d, 1 H); 7.55 (bd, 1 H); 6.85 | | pyridinecarbaldehyde | |
| | (d, 1 H); 6.74 (d, 1 H); 6.67 (d, 1 H), | | (Aldrich) | |
| | 4.32 (t, 2 H); 3.92 (s, 2 H); 2.95 (bd, | | | |
| | 2 H); 2.65 (t, 2 H); 2.61-2.52 (m, 1 H); | | | |
| | 2.23-2.16 (m, 2 H); 1.89 (bd, 2 H); | | | |
| | 1.44-1.37 (m, 2 H). [ES MS] m/z 421 | | | |
| | (MH+). | | | |
| 40 | 1H-NMR (δ, ppm, CDCl₃): 8.42 (d, | Example 1 | 3,5- | Intermediate 21 |
| | 1 H); 7.96 (d, 1 H); 7.88 (d, 1 H); 7.68 | | dichlorobenzaldehyde | |
| | (d, 1 H); 7.55 (bd, 1 H); 6.86 (d, 1 H); | | (Aldrich) | |
| | 4.32 (t, 2 H); 4.01 (s, 3 H); 3.73 (s, | | | |
| | 2 H); 2.96 (bd, 2 H); 2.65 (t, 2 H); 2.55- | | | |
| | 2.47 (m, 1 H); 2.22-2.15 (m, 2 H); 1.90 | | | |
| | (bd, 2 H); 1.46-1.35 (m, 2 H). [ES MS] | | | |
| | m/z 446 (MH+). | | | |
| 41 | ¹H-NMR (δ, ppm, DMSO-d₆): 8.27 (d, | Example 1 | 4- | Intermediate 29 |
| | 1 H); 7.85 (d, 1 H); 7.41-7.38 (m, 4 H); | | fluorobenzaldehyde | |
| | 6.64 (d, 1 H); 4.33 (t, 2 H); 3.96 (s, | | (Acros) | |
| | 3 H); 3.69 (s, 2 H); 2.91-2.87 (m, 2 H); | | | |
| | 2.34-2.25 (m, 1 H); 2.05-1.97 (m, 2 H); | | | |
| | 1.76-1.71 (m, 2 H); 1.25-1.14 (m, 2 H). | | | |
| | [ES MS] m/z 461 (MH+). | | | |
| 42 | ¹H-NMR (δ, ppm, DMSO-d₆): 8.27 (d, | Example 1 | 4- | Intermediate 8 |
| | 1 H); 8.03-8.02 (m, 1 H); 7.87-7.84 (m, | | (trifluoromethyl) | |
| | 2 H); 7.40-7.39 (m, 1 H); 6.65 (d, 1 H); | | benzaldehyde | |
| | 4.33 (t, 2 H); 3.96 (s, 3 H); 3.90 (s, | | (Aldrich) | |
| | 3 H); 3.64 (s, 2 H); 2.92-2.88 (m, 2 H); | | | |
| | 2.36-2.27 (m, 1 H); 2.05-1.98 (m, 2 H); | | | |
| | 1.77-1.73 (m, 2 H); 1.26-1.14 (m, 2 H). | | | |
| | [ES MS] m/z 458 (MH+). | | | |
| 43 | 1H-NMR (δ, ppm, CDCl₃): 8.55 (dd, | Example 1 | 4- | Intermediate 8 |
| | 1 H); 7.91 (d, 1 H); 7.79 (bd, 1 H); 7.46 | | chlorobenzaldehyde | |
| | (dd, 1 H); 6.92 (d, 1 H); 6.88 (d, 1 H); | | (Fluka) | |
| | 6.66 (d, 1 H); 4.39 (t, 2 H); 3.94 (s, | | | |
| | 2 H); 2.98 (bd, 2 H); 2.65 (t, 2 H); 2.57 | | | |
| | (bs, 1 H); 2.23-2.16 (m, 2 H); 1.90 (bd, | | | |
| | 2 H); 1.47-1.35 (m, 2 H). [MS ES] m/z | | | |
| | 447 (MH+). | | | |
| 44 | ¹H-NMR (δ, ppm, CDCl₃): 8.55 (dd, | Example 1 | 5-chloro-2- | Intermediate 21 |
| | 1 H); 7.91 (d, 1 H); 7.79 (bd, 1 H); 7.49- | | thiophenecarbaldehyde | |
| | 7.44 (m, 1 H); 7.37 (s, 1 H); 6.92 (d, | | (Aldrich) | |
| | 1 H); 4.40 (bt, 2 H); 3.98 (s, 2 H); 2.96 | | | |
| | (bd, 2 H); 2.66 (bt, 2 H); 2.60-2.47 (m, | | | |
| | 1 H); 2.18 (bt, 2 H); 1.87 (bd, 2 H); | | | |
| | 1.47-1.31 (m, 2 H). [ES MS] m/z 448 | | | |
| | (MH⁺). | | | |
| 45 | ¹H-NMR (δ, ppm, CDCl₃): 8.26 (d, | Example 1 | 5-chloro-6- | Intermediate 21 |
| | 1 H); 7.85 (d, 1 H); 7.21 (s, 1 H); 6.76- | | (methyloxy)-3- | |
| | 6.72 (m, 2 H); 4.36 (bt, 2 H); 3.98 (s, | | pyridinecarbaldehyde | |
| | 3 H); 3.93 (s, 2 H); 2.98 (bd, 2 H); 2.65 | | (Asymchem) | |
| | (bt, 2 H); 2.60-2.49 (m, 1 H); 2.19 (bt, | | | |
| | 2 H); 1.88 (bd, 2 H); 1.48-1.31 (m, 2 H). | | | |
| | [ES MS] m/z 555 (MH⁺). | | | |
| 46 | ¹H-NMR (δ, ppm, CDCl₃): 8.55 (dd, | Example 1 | 3,5- | Intermediate 29 |
| | 1 H); 7.91 (d, 1 H); 7.79 (bd, 1 H); 7.49- | | dichlorobenzaldehyde | |
| | 7.44 (m, 1 H); 7.34 (d, 1 H); 7.17 (dd, | | (Aldrich) | |
| | 1 H); 6.91 (d, 1 H); 6,87 (d, 1 H); 4.40 | | | |
| | (t, 2 H); 3.89 (s, 3 H); 3.73 (s, 2 H); | | | |
| | 2.96 (bd, 2 H); 2.65 (t, 2 H); 2.59-2.45 | | | |
| | (m, 1 H); 2.19 (bt, 2 H); 1.89 (bd, 2 H); | | | |
| | 1.52-1.33 (m, 2 H). [ES MS] m/z 427 | | | |
| | (MH+). | | | |
| 47 | ¹H-NMR (δ, ppm, CDCl₃): 8.55 (dd, | Example 1 | 5-chloro-6- | Intermediate 29 |
| | 1 H); 7.91 (d, 1 H); 7.78 (bd, 1 H); 7.61 | | (methyloxy)-3- | |
| | (d, 1 H); 7.55 (d, 1 H); 7.49-7.44 (m, | | pyridinecarbaldehyde | |
| | 1 H); 7.13 (dd, 1 H); 6.92 (d, 1 H); 4.39 | | (Asymchem) | |
| | (t, 2 H); 3.75 (s, 2 H); 2.98 (bd, 2 H); | | | |
| | 2.65 (t, 2 H); 2.58-2.44 (m, 1 H); 2.19 | | | |
| | (bt, 2 H); 1.87 (bd, 2 H); 1.49-1.32 (m, | | | |
| | 2 H). [ES MS] m/z 519 (MH+). | | | |
| 48 | ¹H-NMR (δ, ppm, CDCl₃): 8.28 (d, | Example 1 | 5-bromo-2- | Intermediate 8 |
| | 1 H); 7.84 (d, 1 H); 7.61 (d, 2 H); 7.45 | | thiophenecarbaldehyde | |
| | (d, 2 H); 7.21 (s, 1 H); 6.74 (d, 1 H); | | (Aldrich) | |
| | 4.37 (bt, 2 H); 3.98 (s, 3 H); 3.88 (s, | | | |
| | 2 H); 2.97 (bd, 2 H); 2.65 (bt, 2 H); | | | |
| | 2.59-2.45 (m, 1 H); 2.19 (bt, 2 H); 1.89 | | | |
| | (bd, 2 H); 1.52-1.34 (m, 2 H). [ES MS] | | | |
| | m/z 418 (MH+). | | | |
| 49 | ¹H-NMR (δ, ppm, DMSO-d₆): 8.27 (d, | Example 1 | 2-bromo-1,3- | Intermediate 8 |
| | 1 H); 7.86 (bd, 1 H); 7.40-7.39 (m, 1 H); | | thiazole-5- | |
| | 7.00 (d, 1 H); 6.77 (bd, 1 H); 6.65 (d, | | carbaldehyde | |
| | 1 H); 4.33 (t, 2 H); 3.97 (s, 3 H); 3.84 | | (Combi-Blocks) | |
| | (s, 2 H); 2.92-2.88 (m, 2 H); 2.43-2.32 | | | |
| | (m, 1 H); 2.05-1.98 (m, 2 H); 1.77-1.73 | | | |
| | (m, 2 H); 1.24-1.14 (m, 2 H). [ES MS] | | | |
| | m/z 477 (MH+). | | | |
| 50 | ¹H-NMR (δ, ppm, DMSO-d₆): 8.52- | Example 1 | 4,5-dibromo-2- | Intermediate 29 |
| | 8.50 (m, 1 H); 7.99 (d, 1 H); 7.92 (d, | | thiophenecarbaldehyde | |
| | 1 H); 7.62-7.58 (m, 1 H); 6.94 (s, 1 H); | | | |
| | 6.84 (d, 1 H); 4.31 (t, 2 H); 3.83 (s, | | (Acros) | |
| | 2 H); 2.89-2.86 (m, 2 H); 2.38-2.31 (m, | | | |
| | 1 H); 2.04-1.97 (m, 2 H); 1.76-1.71 (m, | | | |
| | 2 H); 1.24-1.13 (m, 2 H). [ES MS] m/z | | | |
| | 525 (MH+). | | | |
| 51 | 1H-NMR (δ, ppm CDCl₃): 8.42 (d, | Example 1 | 3-chloro-4- | Intermediate 8 |
| | 1 H); 7.89 (d, 1 H); 7.55 (bs, 1 H); 6.86 | | (methyloxy) | |
| | (d, 1 H); 6.60 (s, 1 H); 4.33 (bs, 2 H); | | benzaldehyde | |
| | 3.89 (s, 2 H); 2.97-2.95 (m, 2 H); 2.68- | | (Aldrich) | |
| | 2.63 (m, 2 H); 2.57 (bs, 1 H); 2.23-2.18 | | | |
| | (m, 2 H); 2.13 (s, 3 H); 1.91-1.87 (m, | | | |
| | 2 H); 1.41 (bs, 2 H). [MS ES] m/z 479 | | | |
| | (MH+). | | | |
| 52 | ¹H-NMR (δ, ppm, CDCl₃): 8.55 (dd, | Example 1 | 3,4- | Intermediate 8 |
| | 1 H); 7.92 (d, 1 H); 7.81 (bd, 1 H); 7.49- | | dibromobenzaldehyde | |
| | 7.45 (m, 1 H); 7.33 (s, 1 H); 7.28-7.18 | | (Alfa Aesar) | |
| | (m, 3 H); 6.92 (d, 1 H); 4.40 (bt, 2 H); | | | |
| | 3.79 (s, 2 H); 2.97 (bd, 2 H); 2.66 (bt, | | | |
| | 2 H); 2.59-2.46 (m, 1 H); 2.20 (bt, 2 H); | | | |
| | 1.89 (bd, 2 H); 1.53-1.35 (m, 2 H). [ES | | | |
| | MS] m/z 397 (MH+). | | | |
| 53 | 1H-NMR (δ, ppm, CDCl₃): 8.55 (dd, | Example 1 | 4- | Intermediate 29 |
| | 1 H); 7.92 (d, 1 H); 7.79 (bd, 1 H); 7.47 | | formylbenzonitrile | |
| | (dd, 1 H); 7.27-7.23 (m, 3 H); 6.92 (d, | | (Fluka) | |
| | 1 H); 4.39 (t, 2 H); 3.77 (s, 2 H); 2.98 | | | |
| | (bd, 2 H); 2.65 (t, 2 H); 2.54-2.47 (m, | | | |
| | 1 H); 2.23-2.16 (m, 2 H); 1.90 (bd, 2 H); | | | |
| | 1.47-1.34 (m, 2 H). [ES MS] m/z 431 | | | |
| | (MH+). | | | |
| 54 | 1H-NMR (δ, ppm, CDCl₃): 8.42 (d, | Example 1 | 5-bromo-2- | Intermediate 29 |
| | 1 H); 7.95 (d, 1 H); 7.88 (d, 1 H); 7.54 | | thiophenecarbaldehyde | |
| | (bd, 1 H); 7.48 (d, 1 H); 7.28 (d, 1 H); | | (Aldrich) | |
| | 6.85 (d, 1 H); 4.32 (t, 2 H); 3.85 (s, | | | |
| | 2 H); 2.96 (bd, 2 H); 2.65 (t, 2 H); 2.57 | | | |
| | (s, 3 H); 2.55-2.48 (m, 1 H); 2.22-2.15 | | | |
| | (m, 2 H); 1.90 (bd, 2 H); 1.47-1.35 (m, | | | |
| | 2 H). [ES MS] 440 (MH+). | | | |
| 55 | 1H-NMR (δ, ppm, CDCl₃): 8.42 (d, | Example 1 | 4,5-dibromo-2- | Intermediate 8 |
| | 1 H); 7.89 (d, 1 H); 7.53 (bd, 1 H); 7.16 | | thiophenecarbaldehyde | |
| | (s, 1 H); 6.86 (d, 1 H); 4.32 (t, 2 H); | | (Acros) | |
| | 4.12 (s, 2 H); 2.96 (bd, 2 H); 2.65 (t, | | | |
| | 2 H); 2.60-2.54 (m, 1 H); 2.22-2.14 (m, | | | |
| | 2 H); 1.92 (bd, 2 H); 1.47-1.36 (m, 2 H). | | | |
| | [ES MS] m/z 466 (MH+). | | | |
| 56 | ¹H-NMR (δ, ppm, CDCl₃): 8.41 (d, | Example 1 | 5-bromo-4- | Intermediate 21 |
| | 1 H); 7.88 (d, 1 H); 7.53 (dd, 1 H); 7.31 | | methyl-2- | |
| | (d, 1 H); 7.18-7.08 (m, 2 H); 6.85 (d, | | thiophenecarbaldehyde | |
| | 1 H); 4.31 (t, 2 H); 3.75 (s, 2 H); 2.95 | | (FRONTIER) | |
| | (bd, 2 H); 2.64 (t, 2 H); 2.58-2.45 (m, | | | |
| | 1 H); 2.35 (s, 3 H); 2.18 (bt, 2 H); 1.87 | | | |
| | (bd, 2 H); 1.49-1.31 (m, 2 H). [ES MS] | | | |
| | m/z 429 (MH+). | | | |
| 57 | 1H-NMR (δ, ppm, CDCl₃): 8.42 (d, | Example 1 | 3- | Intermediate 8 |
| | 1 H); 7.88 (d, 1 H); 7.83 (d, 1 H); 7.53 | | chlorobenzaldehyde | |
| | (dd, 1 H); 7.38 (dd, 1 H); 6.86 (d, 1 H); | | (Aldrich) | |
| | 4.31 (t, 2 H); 4.01 (s, 3 H); 3.75 (s, | | | |
| | 2 H); 2.95 (bd, 2 H); 2.64 (t, 2 H); 2.55- | | | |
| | 2.45 (m, 1 H); 2.22-2.14 (m, 2 H); 1.89 | | | |
| | (bd, 2 H); 1.45-1.32 (m, 2 H). [ES MS] | | | |
| | m/z 430 (MH+). | | | |
| 58 | 1H-NMR (δ, ppm, CDCl₃): 8.28 (d, | Example 1 | 3,5- | Intermediate 8 |
| | 1 H); 7.83 (d, 1 H); 7.23 (d, 1 H); 6.73 | | dichlorobenzaldehyde | |
| | (d, 1 H); 6.22 (d, 1 H); 6.15 (d, 1 H); | | (Aldrich) | |
| | 4.36 (t, 2 H); 3.97 (s, 3 H); 3.77 (s, | | | |
| | 2 H); 2.98 (bd, 2 H); 2.64 (t, 2 H); 2.56- | | | |
| | 2.46 (m, 1 H); 2.18 (t, 2 H); 1.87 (bd, | | | |
| | 2 H); 1.47-1.34 (m, 2 H). [ES MS] m/z | | | |
| | 461 (MH+). | | | |
| 59 | ¹H-NMR (δ, ppm, CDCl₃): 8.28 (d, | Example 1 | 4-methyl-3- | Intermediate 21 |
| | 1 H); 7.85 (d, 1 H); 7.20 (d, 1 H); 7.16 | | nitrobenzaldehyde | |
| | (s, 1 H); 6.74 (d, 1 H); 4.36 (t, 2 H); | | (Alfa Aesar) | |
| | 4.12 (s, 2 H); 3.98 (s, 3 H); 2.96 (bd, | | | |
| | 2 H); 2.65 (t, 2 H); 2.62-2.52 (m, 1 H); | | | |
| | 2.18 (t, 2 H); 1.90 (bd, 2 H); 1.50-1.32 | | | |
| | (m, 2 H). [ES MS] m/z 478 (MH+). | | | |
| 60 | 1H-NMR (δ, ppm, CDCl₃): 8.55 (dd, | Example 1 | 4-bromo-1,3- | Intermediate 21 |
| | 1 H); 7.90 (d, 1 H); 7.78 (d, 1 H); 7.46 | | thiazole-2- | |
| | (dd, 1 H); 6.91 (d, 1 H); 6.59 (s, 1 H); | | carbaldehyde | |
| | 4.38 (t, 2 H); 3.89 (s, 2 H); 2.96 (bd, | | (Frontier) | |
| | 2 H); 2.64 (t, 2 H); 2.59-2.52 (m, 1 H); | | | |
| | 2.19 (t, 2 H); 2.13 (s, 3 H); 1.88 (bd, | | | |
| | 2 H); 1.50-1.33 (m, 2 H). [ES MS] m/z | | | |
| | 461 (MH+). | | | |
| 61 | 1H-NMR (δ, ppm, CDCl₃): 8.28 (d, | Example 1 | 3-chloro-4- | Intermediate 21 |
| | 1 H); 7.86-7.83 (m, 2 H); 7.38 (dd, 1 H); | | methylbenzaldehyde | |
| | 7.27-7.21 (m, 1 H); 6.74 (d, 1 H); 4.36 | | (Aldrich) | |
| | (t, 2 H); 4.01 (s, 3 H); 3.98 (s, 3 H); | | | |
| | 3.75 (s, 2 H); 2.98 (d, 2 H); 2.65 (t, | | | |
| | 2 H); 2.56-2.46 (m, 1 H); 2.18 (t, 2 H); | | | |
| | 1.90 (bd, 2 H); 1.47-1.34 (m, 2 H). [ES | | | |
| | MS] m/z 442 (MH+). | | | |
| 62 | 1H-NMR (δ, ppm, CDCl₃): 8.28 (d, | Example 1 | 5-fluoro-6- | Intermediate 21 |
| | 1 H); 8.07 (d, 1 H); 7.84 (d, 1 H); 7.57 | | (methyloxy)-3- | |
| | (dd, 1 H); 7.23 (bs, 1 H); 6.76-6.71 (m, | | pyridinecarbaldehyde | |
| | 2 H); 4.37 (bd, 2 H); 3.98 (s, 3 H); 3.93 | | (Asychem) | |
| | (s, 3 H); 3.74 (s, 2 H); 2.99 (bd, 2 H); | | | |
| | 2.65 (bt, 2 H); 2.54 (bs, 1 H); 2.19 (bt, | | | |
| | 2 H); 1.91 (bd, 2 H); 1.51-1.41 (m, 2 H). | | | |
| | [ES MS] m/z 424 (MH+). | | | |
| 63 | 1H-NMR (δ, ppm, CDCl₃): 8.27 (d, | Example 1 | 5-bromo-2- | Intermediate 29 |
| | 1 H); 7.84 (d, 1 H); 7.29-7.23 (m, 2 H); | | furancarbaldehyde | |
| | 7.15-7.11 (m, 1 H); 6.73 (d, 1 H); 4.38 | | (Aldrich) | |
| | (t, 2 H); 3.98 (s, 3 H); 3.87 (s, 2 H); | | | |
| | 3.01 (bd, 2 H); 2.66 (t, 2 H); 2.56 (bs, | | | |
| | 1 H); 2.50 (s, 3 H); 2.20 (bs, 2 H); 1.94 | | | |
| | (bd, 2 H); 1.53-1.41 (m, 2 H). [ES MS] | | | |
| | m/z 426 (MH+), | | | |
| 64 | 1H-NMR (δ, ppm, CDCl₃): 8.27 (d, | Example 1 | 4-bromo-1,3- | Intermediate 29 |
| | 1 H); 7.84 (d, 1 H); 7.31-7.08 (m, 4 H); | | thiazole-2- | |
| | 6.74 (d, 1 H); 4.36 (t, 2 H); 3.97 (s, | | carbaldehyde | |
| | 3 H); 3.75 (s, 2 H); 2.98 (bd, 2 H); 2.64 | | (Frontier) | |
| | (t, 2 H); 2.55-2.47 (m, 1 H); 2.35 (s, | | | |
| | 3 H); 2.18 (bt, 2 H); 1.90 (bd, 2 H); | | | |
| | 1.52-1.36 (m, 2 H). [ES MS] m/z 441 | | | |
| | (MH+). | | | |
| 65 | ¹H-NMR (δ, ppm, CDCl₃): 8.28 (d, 1 H); | Example 1 | 5-bromo-4- | Intermediate 8 |
| | 7.93 (s, 1 H); 7.84 (d, 1 H); 7.62-7.58 | | methyl-2- | |
| | (m, 1 H); 7.23-7.19 (m, 1 H); 6.74 (d, | | thiophenecarbaldehyde | |
| | 1 H); 4.40-4.36 (m, 2 H); 3.98 (s, 3 H); | | (Frontier) | |
| | 3.77 (s, 2 H); 3.03-2.99 (m, 2 H); 2.68- | | | |
| | 2.64 (m, 2 H); 2.56-2.49 (m, 1 H); 2.28 | | | |
| | (s, 3 H); 2.24-2.18 (m, 2 H); 1.95-1.90 | | | |
| | (m, 2 H); 1.49-1.41 (m, 2 H). [ES MS] | | | |
| | m/z 426 (MH+). | | | |
| 66 | ¹H-NMR (δ, ppm, CDCl₃): 8.28 (d, 1 H); | Example 1 | 5-fluoro-6- | Intermediate 29 |
| | 7.85 (d, 1 H); 7.25 (d, 1 H); 6.96-6.87 | | (methyloxy)-3- | |
| | (m, 3 H); 6.75 (d, 1 H); 4.38 (bt, 2 H); | | pyridinecarbaldehyde | |
| | 3.98 (s, 3 H) 3.74 (s, 2 H); 3.04-2.94 | | Asymchem) | |
| | (m, 2 H); 2.65 (bt, 2 H); 2.61-2.50 (m, | | | |
| | 1 H); 2.31 (s, 6 H); 2.20 (dt, 2 H); 1.94 | | | |
| | (bd, 2 H); 1.53-1.36 (m, 2 H). [ES MS] | | | |
| | m/z 421 (MH+). | | | |
| 67 | ¹H-NMR (δ, ppm, CDCl₃): 8.28 (d, 1 H); | Example 1 | 6-(methyloxy)-3- | Intermediate 29 |
| | 7.84 (d, 1 H); 7.25 (d, 1 H); 7.12-7.01 | | pyridinecarbaldehyde | |
| | (m, 3 H); 6.74 (d, 1 H); 4.37 (bt, 2 H); | | (Aldrich) | |
| | 3.98 (s, 3 H) 3.75 (s, 2 H); 3.04-2.93 | | | |
| | (m, 2 H); 2.65 (bt, 2 H); 2.61-2.49 (m, | | | |
| | 1 H); 2.26 (s, 3 H); 2.25 (s, 3 H); 2.19 | | | |
| | (dt, 2 H); 1.93 (bd, 2 H); 1.52-1.35 (m, | | | |
| | 2 H). [ES MS] m/z 421 (MH+). | | | |
| 68 | ¹H-NMR (δ, ppm, CDCl₃): 8.28 (d, | Example 1 | 5-fluoro-6- | Intermediate 29 |
| | 1 H); 7.84 (d, 1 H); 7.25-7.23 (m, 1 H); | | methyl-2- | |
| | 6.90-6.77 (m, 3 H); 6.74 (d, 1 H); 4.37 | | pyridinecarbaldehyde | |
| | (t, 2 H); 3.98 (s, 3 H); 3.76 (s, 2 H); | | (Asymchem) | |
| | 2.99 (bd, 2 H); 2.65 (t, 2 H); 2.56-2.48 | | | |
| | (m, 1 H); 2.33 (s, 3 H); 2.19 (bt, 2 H); | | | |
| | 1.91 (bd, 2 H); 1.49-1.36 (m, 2 H). [ES | | | |
| | MS] m/z 425 (MH+). | | | |
| 69 | ¹H-NMR (δ, ppm, CDCl₃): 8.28 (d, 1 H); | Example 1 | 3-chloro-4- | Intermediate 29 |
| | 7.85 (d, 1 H); 7.25 (bd, 1 H); 7.14-7.09 | | methylbenzaldehyde | |
| | (m, 1 H); 7.00-6.96 (m, 2 H); 6.74 (d, | | (Aldrich) | |
| | 1 H); 4.37 (t, 2 H); 3.98 (s, 3 H); 3.77 | | | |
| | (s, 2 H); 2.99 (bd, 2 H); 2.65 (t, 2 H); | | | |
| | 2.58-2.48 (m, 1 H); 2.25 (d, 3 H); 2.18 | | | |
| | (bd, 2 H); 1.91 (bd, 2 H); 1.49-1.36 (m, | | | |
| | 2 H). [ES MS] m/z 425 (MH+). | | | |
| 70 | ¹H-NMR (δ, ppm, CDCl₃): 8.42 (d, | Example 1 | 6-fluoro-5- | Intermediate 29 |
| | 1 H); 7.88 (d, 1 H); 7.88 (d, 1 H); 7.55 | | methyl-3- | |
| | (dd, 1 H); 7.28 (bs, 1 H); 7.18 (bs, 1 H); | | pyridinecarbaldehyde | |
| | 7.07 (bdd, 1 H); 6.86 (d, 1 H); 4.31 (t, | | (Asymchem) | |
| | 2 H); 3.74 (s, 2 H); 2.95 (bd, 2 H); 2.64 | | | |
| | (t, 2 H); 2.56-2.46 (m, 1 H); 2.18 (t, | | | |
| | 2 H); 1.90 (bd, 2 H); 1.47-1.34 (m, 2 H). | | | |
| | [ES MS] m/z 429 (MH+). | | | |
| 71 | ¹H-NMR (δ, ppm, CDCl₃): 8.47 (bd, | Example 1 | 4-chloro-5-fluoro- | Intermediate 29 |
| | 1 H); 8.42 (d, 1 H); 7.90 (bs, 1 H); 7.90 | | 2- | |
| | (bs, 1 H); 7.87 (bs, 1 H); 7.54 (dd, 1 H); | | pyridinecarbaldehyde | |
| | 6.86 (d, 1 H); 4.32 (t, 2 H); 3.82 (s, | | (Intermediate 48) | |
| | 2 H); 2.96 (bd, 2 H); 2.65 (t, 2 H); 2.57- | | | |
| | 2.47 (m, 1 H); 2.19 (bt, 2 H); 1.91 (bd, | | | |
| | 2 H); 1.46-1.34 (m, 2 H). [ES MS] m/z | | | |
| | 460 (MH+). | | | |
| 72 | ¹H-NMR (δ, ppm, CDCl₃): 8.54 (d, 1 H); | Example 7 | 5-bromo-4- | Intermediate 21 |
| | 7.91 (d, 1 H); 7.80 (m, 1 H); 7.47 (dd, | | methyl-1,3- | |
| | 1 H); 7.10 (s, 1 H); 6.91 (d, 1 H); 6.83 | | thiazole-2- | |
| | (s, 1 H); 4.40 (t, 2 H); 3.97 (s, 2 H); | | carbaldehyde | |
| | 2.98 (m, 2 H); 2.66 (m, 2 H); 2.21 (m, | | (Intermediate 55) | |
| | 2 H); 1.90 (bd, 2 H); 1.25-1.70 (m, 4 H). | | | |
| | [ES MS] m/z 447 (MH+). | | | |
| 73 | ¹H-NMR (δ, ppm, CDCl₃): 8.41 (d, | Example 8 | 3,5- | Intermediate 29 |
| | 1 H); 7.88 (d, 1 H); 7.61 (d, 1 H); 7.54 | | Dimethylbenzaldehyde | |
| | (d, 2 H); 7.13 (dd, 1 H); 6.85 (d, 1 H); | | (Alfa Aesar) | |
| | 4.32 (bt, 2 H); 3.75 (s, 2 H); 2.96 (bd, | | | |
| | 2 H); 2.65 (bt, 2 H); 2.57-2.44 (m, 1 H); | | | |
| | 2.19 (bt, 2 H); 1.89 (bd, 2 H); 1.47-1.31 | | | |
| | (m, 2 H). [ES MS] m/z 537 (MH+). | | | |
| 74 | 1H-NMR (δ, ppm, CDCl₃): 8.42 (d, | Example 8 | 3,4- | Intermediate 29 |
| | 1 H); 7.88 (d, 1 H); 7.55 (bd, 1 H); 6.87 | | Dimethylbenzaldehyde | |
| | (d, 1 H); 6.85 (d, 1 H); 6.66 (d, 1 H); | | (Aldrich) | |
| | 4.33 (t, 2 H); 3.94 (s, 2 H); 2.96 (bd, | | | |
| | 2 H); 2.66 (t, 2 H); 2.57 (bs, 1 H); 2.23- | | | |
| | 2.17 (m, 2 H); 1.90 (bd, 2 H); 1.45-1.35 | | | |
| | (m, 2 H). [ES MS] m/z 465 (MH+). | | | |
| 75 | ¹H-NMR (δ, ppm, CDCl₃): 8.41 (d, | Example 8 | 3-fluoro-5- | Intermediate 29 |
| | 1 H); 7.88 (d, 1 H); 7.61-7.51 (m, 3 H); | | methylbenzaldehyde | |
| | 7.45 (d, 2 H); 6.86 (d, 1 H); 4.33 (bt, | | (Apollo) | |
| | 2 H); 3.87 (s, 2 H); 2.95 (bd, 2 H); 2.66 | | | |
| | (bt, 2 H); 2.60-2.48 (m, 1 H); 2.20 (bt, | | | |
| | 2 H); 1.90 (bd, 2 H); 1.49-1.36 (m, 2 H). | | | |
| | [ES MS] m/z 449 (MH⁺). | | | |
| 76 | ¹H-NMR (δ, ppm, CDCl₃): 8.41 (d, | Example 8 | 4-bromo-3- | Intermediate 29 |
| | 1 H); 7.88 (d, 1 H); 7.55 (bd, 1 H); 7.21- | | methylbenzaldehyde | |
| | 6.99 (m, 3 H); 6.85 (d, 1 H); 4.32 (t, | | (Intermediate 64) | |
| | 2 H); 3.76 (s, 2 H); 2.96 (bd, 2 H); 2.65 | | | |
| | (t, 2 H); 2.57-2.45 (m, 1 H); 2.19 (bt, | | | |
| | 2 H); 1.90 (bd, 2 H); 1.48-1.33 (m, 2 H). | | | |
| | [ES MS] m/z 417 (MH⁺). | | | |
| 77 | ¹H-NMR (δ, ppm, CDCl₃): 8.28 (d, | Example 8 | 5,6-dichloro-3- | Intermediate 29 |
| | 1 H); 7.85 (d, 1 H); 7.61 (d, 1 H); | | pyridinecarbaldehyde | |
| | 7.54 (d, 1 H); 7.11 (dd, 1 H); 6.75 (d, 1 | | (Intermediate 58) | |
| | H); 4.40 (bs, 2 H); 3.99 (s, 3 H); 3.75 (s, 2 H); | | | |
| | 3.01 (bs, 2 H); 2.68 (bs, 2 H); 2.54 (bs, 1 H); | | | |
| | 2.25 (bs, 2 H); 1.90 (bs, 2 H); 1.47 (bs, 2 H); | | | |
| | [ES MS] m/z 549 (MH+) | | | |
| 78 | ¹H-NMR (δ, ppm, CDCl₃): 8.28 (d, | Example 8 | 3-fluoro-5- | Intermediate 29 |
| | 1 H); 7.85 (d, 1 H); 7.58 (d, 2 H); 7.45 | | methylbenzaldehyde | |
| | (d, 2 H); 7.32 (bs, 1 H); 6.73 (d, 1 H); | | (Apollo) | |
| | 4.43 (bs, 2 H); 4.08 (s, 3 H); 3.87 (s, | | | |
| | 2 H); 3.06 (bs, 2 H); 2.71 (bs, 2 H); 2.59 | | | |
| | (bs, 1 H); 2.25 (bs, 2 H); 1.95 (bs, 2 H); 1.39- | | | |
| | 1.58 (m, 2 H). ([ES MS] m/z 461 (MH+) | | | |
| 79 | ¹H-NMR (δ, ppm, CDCl₃): 8.28 (d, 1 | Example 8 | 4-cloro-3- | Intermediate 21 |
| | H); 7.85 (d, 1 H); 7.28 (bs, 1 H); 6.98-7.27 | | methylbenzaldehyde | |
| | (m, 3 H); 6.73 (d, 1 H); 4.41 (bs, 2 H); 3.99 | | (Fluorochem) | |
| | (s, 3 H); 3.77 (S, 2 H); 3.04 (bs, 2 H); 2.70 | | | |
| | (bs, 2 H); 2.55 (bs, 1 H); 2.25 (bs, 2 H); 1.94 | | | |
| | (bs, 2 H); 1.67 (bs, 2 H); 1.48 (bs, 2 H) | | | |
| | [ES MS] | | | |
| 80 | ¹H-NMR (δ, ppm, CDCl₃): 8.28 (d, | Example 8 | 5-bromo-3- | Intermediate 21 |
| | 1 H); 7.91 (d, 1 H); 7.49 (d, 1 H); 4.44 | | pyridinecarbaldehyde | |
| | (s, 1 H); 6.73 (d, 1 H); 4.48 (t, 2 H) 4.0 (s, | | (Aldrich) | |
| | 1 H); 3.90 (s, 2 H); 3.11 (d, 2 H); 2.68 | | | |
| | (t, 2 H); 2.19 (t, 2 H); 2.99 (d, 2 H); 1.42- | | | |
| | 1.76 (m, 2 H). [ES MS] m/z 427 (MH+) | | | |
| 81 | ¹H-NMR (δ, ppm, CDCl₃): 8.26 (d, | Example 9 | 4-bromo-2- | Intermediate 8 |
| | 1 H); 7.84 (d, 1 H); 7.66 (s, 1 H); 7.44 | | thiophenecarbaldehyde | |
| | (s, 2 H); 7.29 (bs, 1 H); 6.73 (d, 1 H); | | (Aldrich) | |
| | 4.43 (t, 2 H); 3.97 (s, 3 H); 3.83 (s, 2 H); | | | |
| | 3.08 (d, 2 H); 2.72 (t, 2 H); 2.51-2.65 | | | |
| | (m, 1 H); 2.32 (t, 2 H); 1.95 (d, 2 H), | | | |
| | 1.41-1.59 (m, 2 H). [ES MS] m/z 495 | | | |
| | (MH+). | | | |
| 82 | ¹H-NMR (δ, ppm, CDCl₃): 8.35 (dd, | Example 10 | 3,4- | Intermediate 20 |
| | 2 H); 6.74 (d, 1 H); 4.39 (bs, 2 H); | | dibromobenzaldehyde | |
| | 3.98 (s, 3 H); 3.86 (s, 2 H); 2.99 (bs, 2 | | (Alfa Aesar) | |
| | H); 2.67 (bs, 2 H); 2.52 (bs, 2 H); 2.32 (bs, | | | |
| | 1 H); 2.17 (bs, 2 H); 1.91 (bs, 2 H); 1.49- | | | |
| | 1.60 (m, 2 H): ([ES MS] m/z 461 (MH+). | | | |
| 83 | ¹H-NMR (δ, ppm, CDCl₃): 8.65 (d, | Example 10 | 5-bromo-2- | Intermediate 20 |
| | 1 H); 8.28 (d, 1 H); 7.9 (dd, 1 H); 7.92 | | thiophenecarbaldehyde | |
| | (d, 1 H); 7.49 (d, 1 H); 7.40 (d, 1 H); | | (Aldrich) | |
| | 6.74 (d, 1 H); 4.49 (t, 2 H); 4.09 (s, | | | |
| | 2 H); 4.03 (s, 3 H); 3.09-3.18 | | | |
| | (m, 2 H); 2.83 (bs, 1 H); 2.70 (t, 2 H); | | | |
| | 2.21 (t, 2 H); 2.04 (d, 2 H); 1.47- | | | |
| | 1.64 (m, 2 H). ([ES MS] m/z 472 (MH+). | | | |
| 84 | ¹H-NMR (δ, ppm, DMSO-d₆): 8.27 (d, | Example 10 | 4- | Intermediate 20 |
| | 1 H); 7.86 (d, 1 H); 7.49 (s, 1 H); 7.40- | | (trifluoromethyl) | |
| | 7.39 (m, 1 H); 6.64 (d, 1 H); 4.33 (t, | | benzaldehyde | |
| | 2 H); 3.96 (s, 3 H); 3.87 (s, 2 H); 2.92- | | (Aldrich) | |
| | 2.88 (m, 2 H); 2.396-2.31 (m, 1 H); | | | |
| | 2.05-1.97 (m, 2 H); 1.76-1.72 (m, 2 H); | | | |
| | 1.24-1.15 (m, 2 H). [ES MS] m/z 434 | | | |
| | (MH+). | | | |
| 85 | ¹H-NMR (δ, ppm, DMSO-d₆): 8.27 (d, | Example 10 | 3,4- | Intermediate 20 |
| | 1 H); 7.85 (d, 1 H); 7.40-7.39 (m, 1 H); | | difluorobenzaldehyde | |
| | 6.72 (s, 1 H); 6.65 (d, 1 H); 4.33 (t, | | (Aldrich) | |
| | 2 H); 3.96 (s, 3 H); 3.79 (s, 2 H); 2.91- | | | |
| | 2.88 (m, 2 H); 2.40-2.30 (m, 1 H); 2.06 | | | |
| | (s, 3 H); 2.03-1.97 (m, 2 H); 1.76-1.72 | | | |
| | (m, 2 H); 1.23-1.15 (m, 2 H). [ES MS] | | | |
| | m/z 491 (MH+). | | | |
| 86 | 1H-NMR (δ, ppm, CDCl₃): 8.43 (d, | Example 1 | 5-bromo-6- | Intermediate 29 |
| | 1 H); 7.90 (d, 1 H); 7.49-7.35 (m, | | methyl-2- | |
| | 2 H); 7.38 (d, 1 H); 7.18 (dd, 1 H); | | pyridinecarbaldehyde- | |
| | 6.87 (d, 1 H); 4.49-4.37 (m, 1 H); 4.30- | | (Intermediate 52) | |
| | 4.17 (m, 1 H); 3.88 (bs, 1 H); 3.83 (d, | | | |
| | 1 H); 3.74 (d, 1 H); 3.13 (bd, 1 H); | | | |
| | 2.85 (bd, 1 H); 2.69 (t, 2 H); | | | |
| | 2.54-2.42 (m, 1 H); 2.3 (d, 1 H); 2.20 | | | |
| | (td, 1 H). [ES MS] m/z 465 (MH+) | | | |
| 87 | 1H-NMR (δ, ppm, CDCl₃): 8.68 (d, | Example 12 | 3,4- | Intermediate 28 |
| | 1H); 8.29 (d, 1 H); 7.90 (d, 1 H); 7.86 | | dibromobenzaldehyde | |
| | (d, 1 H); 7.65 (d, 1 H); 7.09 (d, 1 H); 6.75 | | (Alfa Aesar) | |
| | (d, 1 H); 4.57-4.34 (m, 1 H); 4.28- | | | |
| | 3.94 (m, 1 H); 3.99 (s, 3 H); 3.95 (bs, 1 H); | | | |
| | 3.91(bs, 2 H); 3.17 (d, 1 H); 2.85 (d, 1 H); | | | |
| | 2.75-2.55 (m, 2 H); 2.46-2.44 (m, 1 H); | | | |
| | 2.3 (d, 1 H); 2.18 (td, 1 H). [ES MS] m/z | | | |
| | 478 (MH+) | | | |
| 88 | 1H-NMR (δ, ppm, CDCl₃): 8.29 (d, | Example 12 | 4- | Intermediate 28 |
| | 1H); 7.85 (d, 1 H); 7.45 (d, 1 H); 7.38 | | (trifluoromethyl) | |
| | (d, 1 H); 7.18 (dd, 1 H); 7.12 (d, 1 H); | | benzaldehyde | |
| | 6.75 (d, 1 H); 4.55-4.41 (m, 1 H); 4.32- | | (Aldrich) | |
| | 4.20 (m, 1 H); 3.99 (bs, 1 H); 3.89 | | | |
| | (bs, 1 H); 3.78 (q, 2 H); 3.15 (d, | | | |
| | 1 H); 2.87 (d, 1 H); 2.77-2.63 (m, | | | |
| | 2 H); 2.53-2.44 (m, 1 H); 2.3 (d, 1 H); | | | |
| | 2.21 (td, 1 H). [ES MS] m/z 477 (MH+) | | | |
| 89 | 1H-NMR (δ, ppm, CDCl₃): 8.68 (s, | Example 12 | 3,4- | Intermediate 28 |
| | 1 H); 8.29 (d, 1 H); 7.90 (d, 1 H); 7.86 | | difluorobenzaldehyde | |
| | (d, 1 H); 7.65 (d, 1 H); 7.09 (d, 1 H); | | (Aldrich) | |
| | 6.76 (d, 1 H); 4.57-4.44 (m, 1 H); 4.30- | | | |
| | 4.14 (m, 1 H); 3.99 (s, 3 H); 3.97- | | | |
| | 3.85 (m, 4 H); 3.17 (bd, 1 H); 2.87 | | | |
| | (bd, 1 H); 2.76-2.64 (m, 2 H); 2.52- | | | |
| | 2.40 (m, 1 H); 2.3 (d, 1 H); 2.20 (td, | | | |
| | 1 H). [ES MS] m/z 478 (MH+) | | | |
| 90 | 1H-NMR (δ, ppm, CDCl₃): 8.29 (d, | Example 12 | 3- | Intermediate 28 |
| | 1 H); 7.85 (d, 1 H); 7.45 (d, 1 H); | | chlorobenzaldehyde | |
| | 7.18 (dd, 1 H); 7.11 (d, 1 H); 6.75 | | (Aldrich) | |
| | (d, 1 H); 4.55-4.41 (m, 1 H); 4.33- | | | |
| | 4.18 (m, 1 H); 3.99 (s, 3 H); 3.88 (bs, 1 H); | | | |
| | 3.83 (d, 2 H); 3.74 (d, 1 H); 3.15 (bd, 1 H); | | | |
| | 2.86 (bd, 1 H); 2.74-2.63 (m, 2 H); 2.53- | | | |
| | 2.42 (m, 1 H); 2.3 (d, 1 H); 2.20 (td, 1 H). | | | |
| | [ES MS] m/z 477 (MH+) | | | |
| 91 | 1H-NMR (δ, ppm, CDCl₃): 8.68 (s, | Example 12 | 4-chloro-3- | Intermediate 28 |
| | 1 H); 8.56 (dd, 1 H); 7.97-7.87 (m, | | (trifluoromethyl) | |
| | 2 H); 7.70 (d, 1 H); 7.65 (d, | | benzaldehyde | |
| | 1 H); 7.51-7.43 (m, 1 H); 6.94 | | (Aldrich) | |
| | (d, 1 H); 4.59-4.46 (m, 1 H); 4.33- | | | |
| | 4.20 (m, 1 H); 4.01-3.63 (m, 3 H); | | | |
| | 3.21-3.12 (bd, 1 H); 2.90-2.80 (bd, | | | |
| | 1 H); 2.74-2.66 (m, 2 H); 2.50-2.41 | | | |
| | (m, 1 H); 2.28 (d, 1 H); 2.19 (td, 1 H). | | | |
| | [ES MS] m/z 448 (MH+) | | | |
| 92 | 1H-NMR (δ, ppm, CDCl₃): 8.68 | Example 12 | 2,4- | Intermediate 28 |
| | (s, 1 H); 8.44 (d, 1 H); 7.90 (d, 1 H); | | dichlorobenzaldehyde | |
| | 7.64 (d, 1 H); 7.42 (dd, 1 H); 6.87 (d, | | (Fluka) | |
| | 1 H); 4.53-4.40 (m, 1 H); 4.14-4.27 | | | |
| | (m, 1 H); 4.02-3.84 (m, 3 H); 3.21- | | | |
| | 3.12 (m, 1 H); 2.80-2.90 (m, 1 H); 2.75- | | | |
| | 2.64 (m, 2 H); 2.51-2.42 (m, 1 H); | | | |
| | 2.3 (d, 1 H); 2.20 (td, 1 H). [ES MS] | | | |
| | m/z 466 (MH+) | | | |
| 93 | ¹H-NMR (δ, ppm, DMSO-d₆): 8.55 (m, | Example 12 | 5-bromo-2- | Intermediate 28 |
| | 1 H); 8.01-7.97 (m, 1 H); 7.93 (d, 1 H); | | pyridinecarbaldehyde | |
| | 7.39 (s, 1 H); 7.31-7.23 (m, 3 H); 6.82 (d, | | (Alfaaesar) | |
| | 1 H); 4.31-4.27 (t, 2 H); 3.68 (s, 2 H); | | | |
| | 2.88-2.84 (m, 2 H); 2.34-2.26 (m, 1 H); | | | |
| | 2.02-1.95 (t, 2 H); 1.76-1.71 (m, 2 H); | | | |
| | 1.22-1.10 (m, 2 H). | | | |
| 94 | ¹H-NMR (δ, ppm, DMSO-d₆): 8.55 (d, | Example 12 | 2-chloro-1,3- | Intermediate 28 |
| | 1 H); 7.98 (br d, 1 H); 7.93 (d, 1 H); | | thiazole-5- | |
| | 7.33 (s, 4 H); 6.82 (d, 1 H); 4.29 (t, 2 H); | | carbaldehyde | |
| | 3.67 (s, 2 H); 2.88-2.84 (m, 2 H); 2.34- | | (ABCR) | |
| | 2.25 (m, 1 H); 2.01-1.95 (m, 2 H); 1.75- | | | |
| | 1.71 (m, 2 H); 1.22-1.14 (m, 2 H). | | | |
| | [ES MS] m/z 415 (MH+) | | | |
| 95 | ¹H-NMR (δ, ppm, CDCl₃): 8.27 (d, | Example 12 | 5-bromo-4- | Intermediate 28 |
| | 1 H); 7.91 (d, 1 H); 7.50 (d, 1 H); 7.27 | | methyl-2- | |
| | (d, 1 H); 6.95 (s, 1 H); 6.73 (d, 1 H); | | thiophenecarbaldehyde | |
| | 4.46 (t, 2 H); 4.03 (s, 3 H); 3.96 (s, | | (FRONTIER) | |
| | 2 H); 3.06 (d, 2 H); 2.65 (t, 2 H); 2.5- | | | |
| | 2.61 (m, 1 H); 2.17 (t, 2 H); 1.93 (d, 2 H), | | | |
| | 1.43 (qd, 2 H). . ([ES MS] 477 m/z (MH+). | | | |
| 96 | ¹H-NMR (δ, ppm, CDCl₃) 8.28 (d, 1 H); | Example 12 | 3,4- | Intermediate 31 |
| | 7.91 (d, 1 H); 7.49 (d, 1 H); 6.96 (d, | | dichlorobenzaldehyde | |
| | 1 H); 6.90 (d, 1 H); 6.73 (d, 1 H); | | (Fluka) | |
| | 4.50 (t, 2 H); 4.16 (s, 2 H); 4.03 (s, 3 H); | | | |
| | 3.17 (d, 2 H); 2.83-2.96 (m, 1 H); | | | |
| | 2.76 (t, 2 H); 2.28 (t, 2 H); 2.03 | | | |
| | (d, 2 H); 1.46-1.63 (m, 2 H). | | | |
| | ([ES MS] 433 m/z (MH+) | | | |
| 97 | 1H-NMR (δ, ppm, CDCl3): 8.48 | Example 12 | 5-(2-furanyl)-4H- | Intermediate 33 |
| | (d, 1 H); 7.92-8.0 (m, 2 H); 6.87 (d, | | pyrazole-3- | |
| | 1 H); 6.85 (s, 1 H); 4.42 (t, 2 H); | | carboxylic acid | |
| | 3.91 (s, 2 H); 3.03 (d, 2 H); 2.64 (t, | | (Apollo) | |
| | 2 H); 2.45-2.58 (m, 1 H); 2.16 | | | |
| | (t, 2 H); 1.90 (d, 2 H); 1.30-1.46 | | | |
| | (m, 2 H). ([ES MS] 466 m/z (MH+). | | | |
| 98 | ¹H-NMR (δ, ppm, CDCl₃): 8.49 | Example 12 | 3,4- | Intermediate 33 |
| | (d, 1 H); 7.91-8.01 (m, 2 H); 6.83- | | dichlorobenzaldehyde | |
| | 6.92 (m, 2 H); 4.43 (t, 2 H); 3.97 (s, 2 H); | | (Fluka) | |
| | 3.08 (d, 3 H); 2.69 (t, 2 H); 2.55- | | | |
| | 2.64 (m, 1 H); 2.22 (t, 2 H); 1.94- | | | |
| | (d, 2 H); 1.39-1.53 (m, 2 H). . ([ES MS] | | | |
| | 543 m/z (MH+) | | | |
| 99 | ¹H-NMR (δ, ppm, MeOD₃) 8.48 | Example 12 | 6- | Intermediate 37 |
| | (d, 1 H); 7.91-8.01 (m, 2 H); 7.26 | | (trifluoromethyl)- | |
| | (s, 1 H); 6.94 (s, 1 H); 6.86 (d, 1 H); | | 3- | |
| | 4.42 (t, 2 H); 3.95 (s, 2 H); 3.03 (d, | | pyridinecarbaldehyde | |
| | 2 H); 2.64 (t, 2 H); 2.48-2.60 (m, 1 H); | | (Apollo) | |
| | 2.15 (t, 2 H); 1.91 (d, 2 H); 1.31-1.48 | | | |
| | (m, 2 H). ([ES MS] 465 m/z (MH+). | | | |
| 100 | ¹H-NMR (δ, ppm, CDCl₃): 8.42 (d, | Example 12 | 3,4- | Intermediate 37 |
| | 1 H); 8.31 (d, 1 H); 7.86 (d, 1 H); 7.65 | | dichlorobenzaldehyde | |
| | (d, 1 H); 7.54 (d, 1 H); 6.86 (d, 1 H); | | (Fluka) | |
| | 4.32 (t, 2 H); 3.79 (s, 2 H); 2.96 (bd, | | | |
| | 2 H); 2.65 (t, 2 H); 2.60 (s, 3 H); 2.55- | | | |
| | 2.46 (m, 1 H); 2.18 (bt, 2 H); 1.90 (bd, | | | |
| | 2 H); 1.46-1.33 (m, 2 H). [ES MS] m/z | | | |
| | 430 (MH+). | | | |
| 101 | ¹H-NMR (δ, ppm, CDCl₃): 8.31 (d, | Example 12 | 6- | Intermediate 35 |
| | 1 H); 8.28 (d, 1 H); 7.84 (d, 1 H); 7.66 | | (trifluoromethyl)- | |
| | (d, 1 H); 7.23 (dd, 1 H); 6.74 (d, 1 H); | | 3- | |
| | 4.36 (t, 2 H); 3.98 (s, 3 H); 3.79 (s, | | pyridinecarbaldehyde | |
| | 2 H); 2.99 (bd, 2 H); 2.65 (t, 2 H); 2.60 | | (Apollo) | |
| | (s, 3 H); 2.65-2.48 (m, 1 H); 2.19 (bt, | | | |
| | 2 H); 1.91 (bd, 2 H); 1.48-1.35 (m, 2 H). | | | |
| | [ES MS] m/z 442 (MH+). | | | |
| 102 | ¹H-NMR (δ, ppm, CDCl₃): 8.55 (d, | Example 12 | 6- | Intermediate 31 |
| | 1 H); 8.31 (d, 1 H); 7.92 (d, 1 H); 7.79 | | (trifluoromethyl)-3- | |
| | (d, 1 H); 7.65 (d, 1 H); 7.47 (dd, 1 H); | | pyridinecarbaldehyde | |
| | 6.92 (d, 1 H); 4.39 (t, 2 H); 3.79 (s, | | (Apollo) | |
| | 2 H); 2.98 (bd, 2 H); 2.65 (t, 2 H); 2.60 | | | |
| | (s, 3 H); 2.57-2.47 (m, 1 H); 2.19 (bt, | | | |
| | 2 H); 1.90 (bd, 2 H); 1.47-1.35 (m, 2 H). | | | |
| | [ES MS] m/z 412 (MH+). | | | |
| 103 | ¹H-NMR (δ, ppm, CDCl₃): 8.28 (d, 1 H); | Example 12 | 3- | Intermediate 20 |
| | 7.84 (d, 1 H); 7.19 (d, 1 H); 6.74 (d, | | chlorobenzaldehyde | |
| | 1 H); 4.36 (t, 2 H); 4.02 (d, 2 H); 3.98 | | (Aldrich) | |
| | (s, 3 H); 3.05-2.91 (m, 2 H); 2.65 (t, | | | |
| | 2 H); 2.60-2.49 (m, 1 H); 2.19 (dt, 2 H); | | | |
| | 1.91 (brd, 2 H); 1.49-1.32 (m, 2 H). [ES | | | |
| | MS] m/z 468 (MH+). | | | |
| 104 | 1H-NMR (δ, ppm, CDCl₃): 8.27 (d, 1 H); | Example 12 | 4- | Intermediate 20 |
| | 7.85 (d, 1 H), 7.60 (d, 1 H); 7.48 (d, 1 H); | | chlorobenzaldehyde | |
| | 6.73 (d, 1 H); 3.99 (t, 3 H); 3.96 (s, 3 H); 3.90 | | (Fluka) | |
| | (s, 2 H); 3.05 (d, 2 H); 2.68 (t, 2 H); 2.56 (bs, | | | |
| | 1 H) 2.24 (bs, 2 H); 1.93 (d, 2 H); 1.86- | | | |
| | 1.64 (m, 2 H); 1.42-1.57 (m, 2 H); | | | |
| | [ES MS] m/z 469 (MH+) | | | |
| 105 | ¹H-NMR (δ, ppm, CDCl₃): 8.29 (d, 1 H); | Example 13 | 4-bromo-2- | Intermediate 28 |
| | 7.88-7.77 (m, 3 H); 7.23 (bd,1 H); 6.75 | | thiophenecarbaldehyde | |
| | (d, 1 H); 4.38 (bt, 2 H); 4.26 (s, 2 H); | | (Aldrich) | |
| | 3.99 (s, 3 H); 3.07-2.96 (m, 2 H); 2.73- | | | |
| | 2.53 (m, 3 H); 2.22 (bt, 2 H); 1.95 (bd, | | | |
| | 2 H); 1.56-1.38 (m, 2 H). [ES MS] m/z | | | |
| | 463 (MH+). | | | |
| 106 | ¹H-NMR (δ, ppm, DMSO-d₆): 8.59- | Example 13 | 5-chloro-2- | Intermediate 28 |
| | 8.54 (m, 2 H); 8.01-7.92 (m, 2 H); 7.70 | | thiophenecarbaldehyde | |
| | (d, 1 H); 6.82 (d, 1 H); 4.29 (t, 2 H); | | (Aldrich) | |
| | 3.77 (s, 2 H); 2.88-2.84 (m, 2 H); 2.37- | | | |
| | 2.29 (m, 2 H); 2.03-1.96 (m, 2 H); 1.75- | | | |
| | 1.71 (m, 2 H); 1.22-1.11 (m, 2 H). [ES | | | |
| | MS] m/z 434 (MH+). | | | |
| 107 | ¹H-NMR (δ, ppm, DMSO): 8.60 (d, | Example 13 | 2-bromo-1,3- | Intermediate 20 |
| | 1 H); 8.27 (d, 1 H); 7.85 (d, 1 H); 7.73 | | thiazole-5- | |
| | (d, 1 H); 7.40 (d, 1 H); 6.64 (d, 1 H); | | carbaldehyde | |
| | 4.36-4.31 (t, 2 H); 3.97 (s, 3 H); 3.81 | | (Combi-Blocks) | |
| | (s, 2 H); 2.92-2.89 (m, 2 H); 2.41- | | | |
| | 2.34 (m, 1 H); 2.06-1.98 (m, 2 H); 1.79- | | | |
| | 1.74 (m, 2 H); 1.27-1.16 (m, 2 H) | | | |
| | [ES MS] m/z 446 (MH+) | | | |
| 108 | ¹H-NMR (δ, ppm, CDCl₃): 8.42 (d, | Example 13 | 4,5-dibromo-2- | Intermediate 20 |
| | 1 H); 7.89 (d, 1 H); 7.74 (d, 1 H); 7.56 | | thiophenecarbaldehyde | |
| | (bd, 1 H); 7.04 (d, 1 H); 6.86 (d, 1 H); | | (Acros) | |
| | 4.32 (t, 2 H); 3.85 (s, 2 H); 2.96 (bd, | | | |
| | 2 H); 2.67-2.63 (m, 2 H); 2.64 (s, 3 H); | | | |
| | 2.58-2.49 (m, 1 H); 2.19 (bt, 2 H); 1.92 | | | |
| | (bd, 2 H); 1.50-1.38 (m, 2 H). [ES MS] | | | |
| | m/z 474 (MH+). | | | |
| 109 | 1H-NMR (δ, ppm, CDCl₃): 8.27 (d, | Example 13 | 4-bromo-2- | Intermediate 20 |
| | 1 H); 7.84 (d, 1 H); 7.73 (d, 1 H); 7.26 | | thiophenecarbaldehyde | |
| | (s, 1 H); 7.02 (d, 1 H); 6.73 (d, 1 H); | | (Aldrich) | |
| | 4.39 (bs, 2 H); 3.98 (s, 3 H); 3.85 (s, | | | |
| | 2 H); 3.01 (bd, 2 H); 2.69-2.64 (m, 5 H); | | | |
| | 2.56 (bs, 1 H); 2.22 (bs, 2 H); 1.94 (bd, | | | |
| | 2 H); 1.54-1.47 (m, 2 H). [ES MS] m/z | | | |
| | 486 (MH+). | | | |
| 110 | 1H-NMR (δ, ppm, CDCl₃): 8.42 (d, 1 H); | Example 15 | 6-(bromomethyl)- | Intermediate 29 |
| | 7.88 (d, 1 H); 7.56 (d, 1 H); 6.85 (d, 1 H); | | 3-(methyloxy)-2- | |
| | 4.43 (t, 2 H); 4.03 (s, 2 H); 2.97 (d, 2 H); 2.66 | | nitropyridine | |
| | (t, 2 H); 2.56 (bs, 1 H); 2.35 (s, 3 H); 2.20 (bs, | | (*not an | |
| | 2 H); 1.95 (d, 2 H); 1.63 (bs, 2 H); 1.35- | | aldehyde | |
| | 1.50 (m, 2 H); [ES MS] m/z 480 (MH+) | | (Intermediate 43) | |
| 111 | ¹H-NMR (δ, ppm, CDCl₃): 8.27 (s, 1 H); | Example 15 | 2-(Bromomethyl)- | Intermediate 29 |
| | 7.84 (d, 1 H); 6.63 (d, 1 H); 4.37 (t, | | 6-chloropyrazine | |
| | 2 H); 4.02 (s, 2 H); 3.98 (s, 3 H); 3.04- | | (*not an | |
| | 2.90 (m, 2 H); 2.71-2.51 (m, 3 H); 2.32 | | aldehyde) | |
| | (s, 3 H); 2.29 (s, 3 H); 2.20 (t, 2 H); | | (Intermediate 57) | |
| | 2.00-1.85 (m, 2 H); 1.53-1.34 (m, 2 H). | | | |
| | [ES MS] m/z 428 (MH+). | | | |
| 112 | ¹H-NMR (δ, ppm, CDCl₃): 8.28 (d, | Example 1 | 2-fluoro-5- | Intermediate 29 |
| | 1 H); 7.85 (d, 1 H); 7.67-7.53 | | formyl | |
| | (m, 2 H); 7.23 (bs, 1 H); 7.13 (t, 1 H); | | benzonitrile | |
| | 6.75 (d; 1 H); 4.38 (bt, 2 H); 3.98 (s, | | (Commercial) | |
| | 3 H); 3.81 (S, 2 H); 3.06-2.95 (m, | | | |
| | 2 H); 2.72-2.62 (m, 2 H); 2.58-2.40 | | | |
| | (m, 1 H); 2.28-2.15 (m, 2 H); 1.97- | | | |
| | 1.84 (m, 2 H); 1.50-1.32 (m, 2 H). | | | |
| | [ES MS] m/z 436 (MH+). | | | |
| 113 | ¹H-NMR (δ, ppm, CDCl₃): 8.82 (d, | Example 1 | 2- | Intermediate 29 |
| | 1 H); 8.28 (d, 1 H); 7.84 (d, 1 H); | | (trifluoromethyl)- | |
| | 7.65 (d, 1 H); 7.28-7.27 (m, 1 H); | | 4-pyrimidine | |
| | 6.73 (d, 1 H); 4.43 (t, 2 H); 4.04 (s, | | carbaldehyde) | |
| | 2 H); 3.97 (s, 3 H); 3.10-3.06 (m, | | (Intermediate 65) | |
| | 2 H); 2.72 (t, 2 H); 2.62-2.56 (m, | | | |
| | 1 H); 2.34-2.26 (m, 2 H); 1.97-1.95 | | | |
| | (m, 2 H); 1.59-1.47 (m, 2 H). [ES | | | |
| | MS] m/z 463 (MH+). | | | |
| 114 | ¹H-NMR (d, ppm, CDCl₃): 8.27 (d, | Example 1 | 4,6-dimethyl-2- | Intermediate 29 |
| | 1 H); 7.84 (d, 1 H); 7.28 (s, 1 H); | | pyridinecarbaldehyde | |
| | 6.93 (s, 1 H); 6.85 (s, 1 H); 6.74 (d, | | (Intermediate 66) | |
| | 1 H); 4.37 (t, 2 H); 3.97 (s, 3 H); | | | |
| | 3.84 (s, 2 H); 3.00 (bd, 2 H); 2.65 (t, | | | |
| | 2 H); 2.60-2.48 (m, 1 H); | | | |
| | 2.49 (s, 3 H); 2.29 (s, 3 H); 2.20 (bt, | | | |
| | 2 H); 1.94 (bd, 2 H); 1.56-1.40 (m, | | | |
| | 2 H). [ES MS] m/z 422 (MH+). | | | |
| 115 | ¹H-NMR (d, ppm, CDCl₃): 8.46 (d, | Example 1 | 5-chloro-3- | Intermediate 29 |
| | 1 H); 8.43 (d, 1 H); 8.28 (d, 1 H); | | pyridinecarbaldehyde | |
| | 7.85 (d, 1 H); 7.72 (bt, 1 H); 7.22 (bd, | | (Commercial) | |
| | 1 H); 6.75 (d, 1 H); 4.37 (t, 2 H); | | | |
| | 3.98 (s, 3 H); 3.83 (s, 2 H); 2.99 (bd, | | | |
| | 2 H); 2.65 (t, 2 H); 2.59-2.47 (m, | | | |
| | 1 H); 2.20 (bt, 2 H); 1.92 (bd, 2 H); | | | |
| | 1.50-1.35 (m, 2 H). [ES MS] m/z | | | |
| | 428 (MH+). | | | |
| 116 | ¹H-NMR (d, ppm, CDCl₃): 8.51 (d, | Example 1 | 5-chloro-2- | Intermediate 20 |
| | 1 H); 8.28 (d, 1 H); 7.84 (d, 1 H); | | pyridinecarbaldehyde | |
| | 7.62 (dd, 1 H); 7.31-7.22 (m, 2 H); | | (Commericial) | |
| | 6.74 (d, 1 H); 4.37 (t, 2 H); | | | |
| | 3.97 (s, 3 H); 3.91 (s, 2 H); 2.99 (bd, | | | |
| | 2 H); 2.65 (t, 2 H); 2.58-2.48 (m, | | | |
| | 1 H); 2.19 (br, 2 H); 1.92 (bd, 2 H); | | | |
| | 1.54-1.37 (m, 2 H). [ES MS] m/z | | | |
| | 428 (MH+). | | | |
| 117 | ¹H-NMR (δ, ppm, CDCl₃): 8.28 | Example 1 | 5-chloro-2- | Intermediate 29 |
| | (dd, 2 H); 7.83 (d, 1 H); 7.66 (d, | | pyridinecarbaldehyde | |
| | 1 H); 7.09 (d, 1 H); 6.73 (d, 1 H); | | (Commercial) | |
| | 4.51-4.41 (m, 1 H); 4.28-4.19 (m, | | | |
| | 1 H); 3.97 (s, 3 H); 3.38-3.72 (m, | | | |
| | 3 H); 3.14 (bd, 1 H); 2.85 (bd, 1 H); | | | |
| | 2.73-2.65 (m, 2 H); 2.58 (s, 3 H); | | | |
| | 2.49-2.43 (m, 1 H); 2.28 (d, 1 H); | | | |
| | 2.18 (td, 1 H); 1.72-1.57 (m, 2 H). | | | |
| | [ES MS] m/z 458 (MH+). | | | |
| 118 | ¹H-NMR (δ, ppm, CDCl₃): 8.28 | Example 1 | 5-chloro-6- | Intermediate 33 |
| | (dd, 2 H); 7.83 (d, 1 H); 7.66 (d, | | methyl-3- | |
| | 1 H); 7.09 (d, 1 H); 6.73 (d, 1 H); | | pyridinecarbaldehyde | |
| | 4.51-4.41 (m, 1 H); 4.28-4.19 (m, | | (WO-A-2006/137485) | |
| | 1 H); 3.97 (s, 3 H); 3.38-3.72 (m, | | | |
| | 3 H); 3.14 (bd, 1 H); 2.85 (bd, 1 H); | | | |
| | 2.73-2.65 (m, 2 H); 2.58 (s, 3 H); | | | |
| | 2.49-2.43 (m, 1 H); 2.28 (d, 1 H); | | | |
| | 2.18 (td, 1 H); 1.72-1.57 (m, 2 H). | | | |
| | [ES MS] m/z 458 (MH+). | | | |
| 119 | ¹H-NMR (δ, ppm, CD₃OD): 8.54 (s, | See above | 2-methyl-1H-imidazole- | Intermediate 20 |
| | 1 H); 8.09-7.92 (m, 2 H); 7.59 | | 4-carbaldehyde | |
| | (s, 1 H); 6.90 (d, 1 H); 4.76-4.64 | | (Commercial) | |
| | (m, 2 H); 4.46 (s, 2 H); 4.12-3.97 | | | |
| | (m, 2 H); 3.49-3.73 (m, 3 H); 3.30- | | | |
| | 2.28 (m, 2 H); 2.65 (s, 3 H); 2.57- | | | |
| | 2.37 (m, 2 H); 2.17-2.00 (m, 2 H). | | | |
| | [ES MS] m/z 385 (MH+). | | | |
| 120 | ¹H-NMR (δ, ppm , CDCl₃): 8.42 (d, | Example 1 | 2-fluoro-5- | Intermediate 20 |
| | 1 H); 7.88 (d, 1 H); 7.68-7.48 | | formylbenzonitrile | |
| | (m, 3 H); 7.16 (t, 1 H); 6.86 (d, 1 H); | | (Commercial) | |
| | 4.32 (t, 2 H); 3.81 (s, 2 H); 3.03- | | | |
| | 2.90 (m, 2 H); 2.65 (t, 2 H); 2.58- | | | |
| | 2.44 (m, 1 H); 2.19 (bt, 2 H); 1.95- | | | |
| | 1.84 (m, 2 H); 1.48-1.32 (m, 2 H). | | | |
| | [ES MS] m/z 424 (MH+). | | | |
| 121 | ¹H-NMR (δ, ppm, CDCl₃): 8.41 (d, | Example 1 | 4-(1,1-dimethylethyl) | Intermediate 20 |
| | 1 H); 7.87 (d, 1 H); 7.55 (dd, 1 H); | | benzaldehyde | |
| | 7.35-7.32 (m, 2 H); 7.26-7.23 (m, | | (Commercial) | |
| | 2 H); 6.84 (d, 1 H); 4.32 (t, 2 H); | | | |
| | 3.77 (s, 2 H); 2.96 (bd, 2 H); 2.64 | | | |
| | (t, 2 H); 2.59-2.52 (m, 1 H); 2.19 (t, | | | |
| | 2 H); 1.92 (bd, 2 H); 1.51-1.36 (m, | | | |
| | 2 H); 1.30 (m, 9 H). [ES MS] m/z | | | |
| | 437 (MH+). | | | |
| 122 | ¹H-NMR (d, ppm, CDCl₃): 8.42 (d, | Example 1 | 6-fluoro-5-methyl-3- | Intermediate 20 |
| | 1 H); 7.93 (bs, 1 H); 7.89 (d, 1 H); | | pyridinecarbaldehyde | |
| | 7.60 (dd, 1 H); 7.54 (dd, 1 H); 6.86 (d, | | (Commercial) | |
| | 1 H); 4.32 (t, 2 H); 3.76 (s, 2 H); | | | |
| | 2.96 (bd, 2 H); 2.65 (t, 2 H); 2.58- | | | |
| | 2.46 (m, 1 H); 2.28 (s, 3 H); 2.18 (bt, | | | |
| | 2 H); 1.90 (bd, 2 H); 1.48-1.32 (m, | | | |
| | 2 H). [ES MS] m/z 414 (MH+). | | | |
| 123 | ¹H-NMR (δ, ppm, DMSO-d₆): | Example 1 | 5-chloro-6- | Intermediate 31 |
| | 8.55 (d, 1 H); 8.39 (s, 1 H); 8.03 (dd, | | methyl-3- | |
| | 1 H); 7.94 (d, 1 H); 7.89 (s, 1 H); | | pyridinecarbaldehyde | |
| | 6.82 (d, 1 H); 4.62 (bs, 1 H); 4.30 (t, | | (WO-A-2006/137485) | |
| | 2 H); 3.85 (s, 2 H); 3.77 (bs, 1 H); | | | |
| | 2.91-2.63 (m, 3 H); 2.63-2.54 (m, | | | |
| | 1 H); 2.51 (s, 3 H); 2.40-2.28 (m, | | | |
| | 1 H); 2.28-2.13 (m, 1 H); 1.73- | | | |
| | 1.47 (m, 2 H) [ES MS] m/z 446 (MH⁺). | | | |
| 124 | ¹H-NMR (d, ppm, CDCl₃): 8.42 (d, | Example 1 | 5-chloro-6-methyl-3- | Intermediate 31 |
| | 1 H); 8.24 (bd, 1 H); 7.88 (d, 1 H); | | pyridinecarbaldehyde | |
| | 7.55 (dd, 1 H); 7.40 (bs, 1 H); 6.86 (d, | | (WO-A-2006137485 | |
| | 1 H); 4.32 (t, 2 H); 3.75 (s, 2 H); | | | |
| | 2.96 (bd, 2 H); 2.65 (t, 2 H); 2.59- | | | |
| | 2.45 (m, 1 H); 2.48 (s, 3 H); | | | |
| | 2.27 (s, 3 H); 2.19 (bt, 2 H); 1.91 (bd, | | | |
| | 2 H); 1.50-1.33 (m, 2 H). [ES MS] | | | |
| | m/z 410 (MH+). | | | |
| 125 | ¹H-NMR (d, ppm, CDCl₃): 8.42 (d, | Example 1 | 5,6-dimethyl-3- | Intermediate 20 |
| | 1 H); 8.24 (bd, 1 H); 7.88 (d, 1 H); | | pyridinecarbaldehyde | |
| | 7.55 (dd, 1 H); 7.40 (bs, 1 H); 6.86 (d, | | (Intermediate 68) | |
| | 1 H); 4.32 (t, 2 H); 3.75 (s, 2 H); | | | |
| | 2.96 (bd, 2 H); 2.65 (t, 2 H); 2.59- | | | |
| | 2.45 (m, 1 H); 2.48 (s, 3 H); 2.27 (s, | | | |
| | 3 H); 2.19 (bt, 2 H); 1.91 (bd, 2 H); | | | |
| | 1.50-1.33 (m, 2 H). [ES MS] m/z 410 | | | |
| | (MH+). | | | |
| 126 | ¹H-NMR (δ, ppm, CDCl₃): 8.42 (d, | Example 1 | 5-bromo-6- | Intermediate 20 |
| | 1 H); 8.35 (d, 1 H); 7.89 (d, 1 H); | | methyl-3- | |
| | 7.84 (s, 1 H); 7.72-7.51 (m, 1 H); | | pyridinecarbaldehyde | |
| | 6.86 (d, 1 H); 4.37 (bs, 2 H); 3.78 (s, | | (Intermediate 69) | |
| | 2 H); 3.12-2.91 (m, 2 H); 2.79- | | | |
| | 2.67 (m, 2 H); 2.65 (s, 3 H); 2.61- | | | |
| | 2.45 (m, 1 H); 2.35-2.13 (m, 2 H); | | | |
| | 2.04-1.83 (m, 2 H); 1.67-1.36 (m, | | | |
| | 2 H). [ES MS] m/z 474 (MH⁺). | | | |
| 127 | ¹H-NMR (d, ppm, CDCl₃): 8.42 (d, | Example 1 | 5-chloro-6- | Intermediate 20 |
| | 1 H); 8.35 (bd, 1 H); 7.89 (d, 1 H); | | ethyl-3- | |
| | 7.66 (bd, 1H); 7.57 (bd, 1 H); 6.86 (d, | | pyridinecarbaldehyde | |
| | 1 H); 4.34 (t, 2 H); 3.79 (s, 2 H); | | (Intermediate 67) | |
| | 2.97 (bd, 2 H); 2.95 (q, 2 H); 2.66 (t, | | | |
| | 2 H); 2.60-2.48 (m, 1 H); 2.21 (bt, | | | |
| | 2 H); 1.92 (bd, 2 H); 150-1.34 (m, | | | |
| | 2 H); 1.29 (t, 3 H). [ES MS] m/z 444 | | | |
| | (MH+). | | | |
| 128 | ¹H-NMR (δ, ppm, CDCl₃): 8.44 (d, | Example 1 | 5-fluoro-6-methyl-3- | Intermediate 31 |
| | 1 H); 8.24 (d, 1 H); 7.90 (d, 1 H); | | pyridinecarbaldehyde | |
| | 7.57-7.50 (m, 2 H); 6.87 (d, 1 H); | | (Intermediate 70) | |
| | 4.55-4.46 (m, 1 H); 4.30-4.21 (m, | | | |
| | 1 H); 4.01 (bs, 1 H); 3.83 (q, 2 H); | | | |
| | 3.23 (bd, 1 H); 2.93 (bd, 1 H); 2.76 | | | |
| | (bs, 2 H); 2.62-2.54 (m, 1 H); 2.51 | | | |
| | (s, 3 H); 2.41-2.28 (m, 2 H); 1.85- | | | |
| | 1.70 (m, 2 H). [ES MS] m/z 430 (MH+). | | | |
| 129 | ¹H-NMR (δ, ppm, CDCl₃): 8.42 (d, | Example 1 | 5,6-dichloro-3- | Intermediate 20 |
| | 1 H); 8.28 (d, 1 H); 7.88 (d, 1 H); | | pyridinecarbaldehyde | |
| | 7.83 (d, 1 H); 7.55 (d, 1 H); 6.85 (d, | | (Intermediate 71) | |
| | 1 H); 4.32 (t, 2 H); 3.81 (s, 2 H); | | | |
| | 2.96 (db, 2 H); 2.65 (t, 2 H); 2.54- | | | |
| | 2.48 (m, 1 H); 2.20 (t, 2 H); 1.90 | | | |
| | (bd, 2 H); 1.46-1.34 (m, 2 H). [ES | | | |
| | MS] m/z 450 (MH⁺). | | | |
| 130 | ¹H-NMR (δ, ppm, CD₃OD): 8.49 | Example 1 | 5-bromo-6- | Intermediate 31 |
| | (d, 1 H); 8.37 (d, 1 H); 8.05-7.96 | | methyl-3- | |
| | (m, 3 H); 6.86 (d, 1 H); 4.45-4.33 | | pyridinecarbaldehyde | |
| | (m, 2 H); 3.91 (bs, 1 H); 3.85-3.73 | | (Intermediate 69) | |
| | (m, 2 H); 3.07-3.02 (m, 1 H); 2.91- | | | |
| | 2.87 (m, 1 H); 2.68-2.54 (m, 3 H); | | | |
| | 2.61 (s, 3 H); 2.34 (bd, 1 H); 2.27- | | | |
| | 2.19 (m, 1 H); 1.71-1.67 (m, 2 H). | | | |
| | [ES MS] m/z 490 (MH+). | | | |
| 131 | ¹H-NMR (δ, ppm, CDCl₃): 8.70 (s, | As described | 4-methyl-N-{[6- | Intermediate 20 |
| | 1 H); 8.42 (d, 1 H); 7.93-7.87 (m, | above. | (trifluoromethyl)- | |
| | 2 H); 7.63 (d, 1 H); 7.55 (bd, 1 H); | | 3-pyridinyl] | |
| | 6.86 (d, 1 H); 4.35 (t, 2 H); 3.79 (s, | | methyl}-4- | |
| | 2 H); 2.71-2.62 (m, 6 H); 1.65 (bs, | | piperidinamine | |
| | 4 H); 1.18 (s, 3 H). ([ES MS] m/z | | | |
| | 464 (MH⁺). | | | |
| 132 | ¹H-NMR (δ, ppm, CDCl₃): 8.29 (d, | As described | N-[(3,4- | Intermediate 26 |
| | 1 H); 7.85 (d, 1 H); 7.47 (s, 1 H); | above | dichlorophenyl) | |
| | 7.37 (d, 1 H); 7.27 (bs, 1 H); 7.19 | | methyl]-4-methyl-4- | |
| | (dd, 1 H); 6.74 (d, 1 H); 4.43 (bs, | | piperidinamine | |
| | 2 H); 4.00 (bs, 3 H); 3.65 (s, 2 H); | | | |
| | 2.70 (bs, 5 H); 1.67 (bs, 3 H); 1.52 | | | |
| | (bs, 2 H); 1.18 (s, 3 H). ([ES MS] | | | |
| | m/z 475 (MH⁺). | | | |
| 133 | ¹H-NMR (δ, ppm, CDCl₃): 8.42 (d, | As example | N-[(3,4- | Intermediate 18 |
| | 1 H); 7.88 (d, 1 H); 7.58 (db, 1 H); | 132 | dichlorophenyl) | |
| | 7.47 (d, 1 H); 7.36 (d, 1 H); 7.18 | | methyl]-4-methyl-4- | |
| | (dd, 1 H); 6.85 (d, 1 H); 4.35 (t, | | piperidinamine | |
| | 2 H); 3.64 (s, 2 H); 2.72-2.62 (m, | | | |
| | 6 H); 1.64 (bs, 4 H); 1.16 (s, 3 H). | | | |
| | ([ES MS] m/z 463 (MH⁺). | | | |
| 134 | ¹H-NMR (δ, ppm, CDCl₃): 8.70 (bs, | As Example | 4-methyl-N-{[6- | Intermediate 20 |
| | 1 H); 8.29 (d, 1 H); 7.91 (db, 1 H); | 132 | (trifluoromethyl)- | |
| | 7.86 (d, 1 H); 7.64 (d, 1 H); 7.27 | | 3-pyridinyl] | |
| | (bs, 1 H); 6.74 (d, 1 H); 4.43 (bs, | | methy}-4- | |
| | 2 H); 4.01 (bs, 3 H); 3.80 (s, 2 H); | | piperidinamine | |
| | 2.70 (bs, 5 H); 1.68 (bs, 3 H); 1.55 | | | |
| | (bs, 2 H); 1.21 (s, 3 H). ([ES MS] | | | |
| | m/z 476 (MH⁺). | | | |

Biological Activity

General Antimicrobial Activity Assay

Whole-cell antimicrobial activity was determined by broth microdilution using the Clinical and Laboratory Standards Institute (CLSI) recommended procedure, Document M7-A7, “Methods for Dilution Susceptibility Tests for Bacteria that Grow Aerobically”. The compounds were tested in serial two-fold dilutions ranging from 0.016 to 64 μg/mL.

Compounds were evaluated against gram-positive organisms, selected from i) Staphylococcus aureus, ii) Streptococcus pneumoniae, and iii) Enterococcus faecalis. Two compounds were additionally evaluated against Streptococcus pyogenes, and Enterococcus faecium.

In addition, compounds were evaluated against gram-negative organisms selected from iv) Haemophilus influenzae, v) Moraxella catarrhalis and vi) Escherichia coll. Two compounds were additionally evaluated against Pseudomonas aeruginosa, Proteus mirabilis, Enterobacter cloacae, Enterobacter aerogenes, Klebsiella pneumoniae and Stenotrophomonas maltophilia.

The minimum inhibitory concentration (MIC) was determined as the lowest concentration of compound that inhibited visible growth. A mirror reader was used to assist in determining the MIC endpoint.

Results of the General Antimicrobial Activity Assay

Examples 1, 1b, 2, 2b, 3, 3b, 4, 4b, 5-8, 8b, 9-11, 11c, 12, 12b, 12c, 13-54, 56-71, 73-86 89-109, 111-125 and 127-133 were tested against organisms i)-vi) listed above in the antimicrobial activity assay. Examples 23 and 82 were additionally tested against the other organisms listed above. All tested Examples showed an MIC value of 2μg/ml or lower against a strain of at least one of the organisms listed above, with the exception of Example 14 and 57 which showed an MIC value of 4 against a strain of at least one of the organisms listed above; Examples 60 and 128 which showed an MIC value of 32 against a strain of at least one of the organisms listed above; Example 119 which showed an MIC value of >64 against a strain of at least one of the organisms listed above; and Example 58 which showed no activity against organisms i)-vi). For at least one strain of each organism i)-vi) listed hereinabove, at least one tested Example had an MIC value of 2μg/ml or lower.

Mycobacterium tuberculosis H37Rv Inhibition Assay

The measurement of the minimum inhibitory concentration (MIC) for each tested compound was performed in 96 wells flat-bottom, polystyrene microtiter plates. Ten two-fold drug dilutions in neat DMSO starting at 400u.M were performed. Five u.I of these drug solutions were added to 95 μl of Middlebrook 7H9 medium. (Lines A-H, rows 1-10 of the plate layout). Isoniazid was used as a positive control, 8 two-fold dilution of Isoniazid starting at 160 μgml⁻¹ was prepared and 5 μl of this control curve was added to 95 μl of Middlebrook 7H9 medium (Difco catalogue ref. 271310). (Row 11, lines A-H). Five μl of neat DMSO were added to row 12 (growth and Blank controls).

The inoculum was standardised to approximately 1×10⁷ cfu/ml and diluted 1 in 100 in Middlebrook 7H9 broth (Middlebrook ADC enrichment, a dehydrated culture media which supports growth of mycobacterial species available from Becton Dickinson Catalogue Ref. 211887), to produce the final inoculum of H37Rv strain (ATCC25618). One hundred μl of this inoculum was added to the entire plate but G-12 and H-12 wells (Blank controls). All plates were placed in a sealed box to prevent drying out of the peripheral wells and they were incubated at 37° C. without shaking for six days. A resazurin solution was prepared by dissolving one tablet of resazurin (Resazurin Tablets for Milk Testing; Ref 330884Y VWR International Ltd) in 30 ml sterile PBS (phosphate buffered saline). 25 μl of this solution was added to each well. Fluorescence was measured (Spectramax M5 Molecular Devices, Excitation 530 nm, Emission 590 nm) after 48 hours to determine the MIC value.

Results of the Mycobacterium tuberculosis H37Rv Inhibition Assay

Examples 1, 1b, 2, 2b, 3, 3b, 4, 4b, 5-8, 8b, 9-11, 11c, 12, 12b, 12c and 13-111 were tested in the Mycobacterium tuberculosis H37Rv inhibition assay. Examples 1, lb, 2, 2b, 3, 3b, 4, 4b, 5-8, 8b, 9-11, 11b, 11c, 12, 12b, 12c, 13-59, 61-118 and 120-135 showed an MIC value of 2.4 μg/ml or lower. Examples 1, 1b, 2, 2b, 3, 3b, 4, 4b, 5, 7-8, 8b, 10, 11, 11b, 11c, 12, 12b, 12c, 13, 15-33, 35-37, 39-47, 50, 52-56, 58, 59, 61-63, 65-67, 69-80, 82-102, 104-110, 112, 115, 117, 118, 120, 122, 123, 124, 126, 127, 129 and 133-135 showed an MIC value of 1.0 u.g/mlor lower.

1. A compound of Formula (I) or a pharmaceutically acceptable salt, solvate or N-oxide thereof: 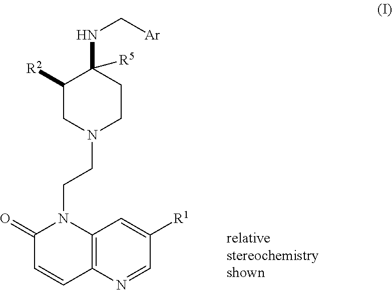
Wherein in Formula (I):
R¹ represents hydrogen; halo; or C₁₋₃alkoxy-;
R² represents hydrogen or hydroxy;
Ar represents a group selected from: phenyl, pyridyl, pyridazinyl, pyrimidinyl, pyrazinyl, thiazolyl, furanyl, imidazolyl and thiophenyl;
wherein
Ar is substituted by a first group R³, wherein R³ represents a group selected from: halo, CF₃, C₁₋₅ alkyl, C₁₋₅ hydroxyalkyl, nitro and cyano; when Ar represents pyridyl, R³ alternatively represents C₁₋₃alkoxy-;
Ar is optionally substituted by a second group R⁴;
when R³ represents halo, the optional R⁴ group represents halo;
when R³ represents CF₃, the optional R⁴ group represents halo;
when R³ represents C₁₋₃alkyl, or C₁₋₅ hydroxyalkyl the optional R⁴ group is selected from halo, CF₃, C₁₋₃alkyl, nitro and C₁₋₃alkoxy-;
when R³ represents nitro, the optional R⁴ group is selected from halo and CF₃;
when R₃ represents cyano, the optional R⁴ group is selected from halo, CF₃, C₁₋₃alkyl and nitro;
when R³ represents C₁₋₃alkoxy-, the optional R⁴ group is selected from halo and nitro;
if R² is hydrogen then R⁵ is hydrogen or C₁₋₃ alkyl, and if R² is hydroxyl then R⁵ is hydrogen.

2. A compound of Formula (I) having the formula (IA), or a pharmaceutically acceptable salt, solvate or N-oxide thereof: 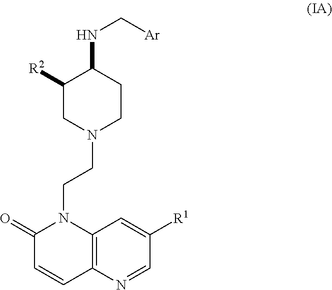
Wherein in Formula (IA):
R¹ represents hydrogen; halo; or C₁₋₃alkoxy-;
R² represents hydrogen or hydroxy;
Ar represents a group selected from: phenyl, pyridyl, pyridazinyl, pyrimidinyl, pyrazinyl, thiazolyl, furanyl, imidazolyl and thiophenyl;
wherein
Ar is substituted by a first group R³, wherein R³ represents a group selected from: halo, CF₃, C₁₋₃alkyl, nitro and cyano; when represents pyridyl, R³ alternatively represents C₁₋₃ alkoxy-;
Ar is optionally substituted by a second group R⁴;
when R³ represents halo, the optional R⁴ group represents halo;
when R³ represents CF₃, the optional R⁴ group represents halo;
when R³ represents C₁₋₃alkyl, the optional R⁴ group is selected from halo, CF₃, C₁₋₃ alkyl, nitro and C alkoxy-;
when R³ represents nitro, the optional R⁴ group is selected from halo and CF₃;
when R₃ represents cyano, the optional R⁴ group is selected from halo, CF₃, C₁₋₃alkyl and nitro;
when R³ represents C₁₋₃alkoxy-, the optional R⁴ group is selected from halo and nitro.

3. A compound of Formula (I) or (IA) or a pharmaceutically acceptable salt, solvate or N-oxide thereof according to claim 1, wherein Ar represents a group selected from: phenyl, pyridyl, pyridazinyl, pyrazinyl, thiazolyl, furanyl, and thiophenyl.

4. A compound of Formula (I), or a pharmaceutically acceptable salt, solvate or N-oxide thereof, according to claim 1, substituent R⁴ is present.

5. A compound of Formula (I) or (IA) according to claim 1 wherein when Ar represents phenyl, the substituent R³ and optional substituent R⁴ are in the meta- or para- position relative to the point of attachment of Ar to the remainder of the molecule.

6. A compound of Formula (I) according to claim 1 wherein, the absolute stereochemistry of the compound of Formula (I) is:

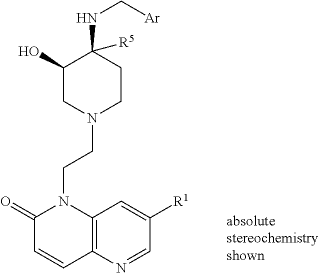

7. A compound of formula (I) according to claim 1 wherein R³ represents halo and the optional R⁴ group represents halo.

8. A compound of Formula (I) according to claim 1 wherein R³ represents CF₃ and R⁴ represents halo.

9. A compound of Formula (I) according to claim 1 wherein R³ represents C₁₋₃alkyl and R⁴ group is selected from halo, C₁₋₃alkyl and nitro.

10. A compound of Formula (I) according to claim 1 wherein R³ represents C₁₋₃alkoxy- and R⁴ is selected from halo and nitro.

11. A compound of Formula (I) according claim 1 wherein Ar is phenyl, R³ is CF₃, and R⁴ is absent, or is present and is methyl or chloro.

12. A compound of Formula (I) according to claim 1 wherein Ar is phenyl, R³ is chloro and R⁴ is absent or is present and is chloro.

13. A compound of Formula (I) according to claim 1 wherein Ar is pyridyl, R³ is CF₃, and R⁴ is absent.

14. A compound of Formula (I) according to claim 1 wherein Ar is pyridyl, R³ is chloro, and R⁴ is present and is methyl, methoxy or fluoro.

15. A compound of Formula (I) according to claim 1 wherein Ar is thienyl, R³ is bromo, and R⁴ is present and is bromo or methyl.

16. A pharmaceutically acceptable salt of a compound of Formula (I) according to claim 1.

17. A pharmaceutically acceptable salt of a compound of Formula (I) according to claim 1 selected from the list:
1-[2-(4-{[(5-bromo-2-pyridinyl)methyl]amino}-1-piperidinyl)ethyl]-7-fluoro-1,5-naphthyridin-2(1H)-one;
1-[2-(4-{[(5-bromo-2-pyridinyl)methyl]amino}-1-piperidinyl)ethyl]-7-fluoro-1,5-naphthyridin-2(1H)-one dihydrochloride;
1-[2-(4-{[(2-bromo-1,3-thiazol-5-yl)methyl]amino}-1-piperidinyl)ethyl]-7-(methyloxy)-1,5-naphthyridin-2(1H)-one;
1-[2-(4-{[(2-bromo-1,3-thiazol-5-yl)methyl]amino}-1-piperidinyl)ethyl]-7-(methloxy)-1,5-naphthyridin-2(1H)-one hydrochloride;
1-[2-(4-{[(3,4- dichlorophenyl)methyl]amino}-1-piperidinyl)ethyl]-7-fluoro-1,5-naphthyridin-2(1H)-one;
1-[2-(4-{[(3,4-dichlorophenyl)methyl]amino}-1-piperidinyl)ethyl]-7-fluoro-1,5-naphthyridin-2(1H)-one;
7-(methyloxy)-1-{2-[4-({[6-(trifluoromethyl)-3-pyridinyl]methyl}amino)-1-piperidinyl]ethyl}-1,5-naphthyridin-2(1H)-one;
7-(methyloxy)-1-{2-[4-({[6-(trifluoromethyl)-3-pyridinyl]methyl}amino)-1-piperidinyl]ethyl}-1,5-naphthyridin-2(1H)-one hydrochloride;
1-[2-(4-{[(4-fluoro-3-methylphenyl)methyl]amino}-1-piperidinyl)ethyl]-7-(methyloxy)-1,5-naphthyridin-2(1H)-one;
1-[2-(4-{[(4,5-dimethyl-1,3-thiazol-2-yl)methyl]amino}-1-piperidinyl)ethyl]-7-(methyloxy)-1,5-naphthyridin-2(1H)-one;
1-[2-(4-{[(4,5-dichloro-1,3-thiazol-2-yl)methyl]amino}-1-piperidinyl)ethyl]-7-(methyloxy)-1,5-naphthyridin-2(1H)-one;
1-[2-(4-{[(3,4-dichlorophenyl)methyl]amino}-1-piperidinyl)ethyl]-1,5-naphthyridin-2(1H)-one;
1-[2-(4-{[(3,4-dichlorophenyl)methyl]amino}-1-piperidinyl)ethyl]-1,5-naphthyridin-2(1H)-one dihydrochloride;
1-[2-(4-{[(6-bromo-3-pyridinyl)methyl]amino}-1-piperidinyl)ethyl]-1,5-naphthyridin-2(1H)-one;
7-fluoro-1-{2-[4-({[6-(trifluoromethyl)-3-pyridinyl]methyl}amino)-1-piperidinyl]ethyl}-1,5-naphthyridin-2(1H)-one;
1-[2-(4-{[(3,4-dichlorophenyl)methyl]amino}-1-piperidinyl)ethyl]-7-(methloxy)-1,5-naphthyridin-2(1 14)-one;
1-[2-(4-{[(3,4-dichlorophenyl)methyl]amino}-1-piperidinyl)ethyl]-7-(methyloxy)-1,5-naphthyridin-2(1H)-one hydrochloride;
1-[2-(4-{[(3,4-dichlorophenyl)methyl]amino}-1-piperidinyl)ethyl]-7-(methyloxy)-1,5-naphthyridin-2(114)-one dihydrochloride;
1-[2-(4-{[(4-chlorophenyl)methyl]amino}-1-piperidinyl)ethyl]-7-(methyloxy)-1,5-naphthyridin-2(1H)-one;
1-[2-(4-{[(4-chlorophenyl)methyl]amino}-1-piperidinyl)ethyl]-7-(methyloxy)-1,5-naphthyridin-2(1H)-one dihydrochloride;
1-[2-(4-{[(4-chlorophenyl)methyl]amino}-1-piperidinyl)ethyl]-7-(methyloxy)-1,5-naphthyridin-2(1H)-one hydrochloride;
1-[2-(4-{[(6-bromo-3-pyridinyl)methyl]amino}-1-piperidinyl)ethyl]-7-fluoro-1,5-naphthyridin-2(1H)-one ;
7-fluoro-1-[2-(4-{[(4-fluorophenyl)methyl]amino}-1-piperidinyl)ethyl]1,5-naphthyridin-2(1H)-one;
7-(methyloxy)-1-{2-[4-({[6-(trifluoromethyl)-3-pyridazinyl]methyl}amino)-1-piperidinyl]ethyl}-1,5-naphthyridin-2(1H)-one;
1-[2-(4-{[(5-Bromo-3-isothiazolyl)methyl]amino}-1-piperidinyl)ethyl]-7-(methyloxy)-1,5-naphthyridin-2(1H)-one;
1-[2-(4-{[(4-chlorophenyl)methyl]amino}-1-piperidinyl)ethyl]-7-(ethyloxy)-1,5-naphthyridin-2(1H)-one;
7-(ethyloxy)-1-{2-[4-({[6-(trifluoromethyl)-3-pyridinyl]methyl}amino)-1-piperidinyl]ethyl}-1,5-naphthyridin-2(1H)-one;
1-[2-(4-{[(3,4-dichlorophenyl)methyl]amino}-1-piperidinyl)ethyl]-7-(ethyloxy)-1,5-naphthyridin-2(1H)-one;
1-[2-(4-{[(5-chloro-6-methyl-3-pyridinyl)methyl]amino}-1-piperidinyl)ethyl]-7-fluoro-1,5-naphthyridin-2(1 11)-one;
1-[2-(4-{[(4-bromo-2-pyridinyl)methyl]amino}-1-piperidinyl)ethyl]-7-(methoxy)-1,5-naphthyridin-2(1H)-one;
1-[2-(4-{[(5-bromo-3-pyridinyl)methyl]amino}-1-piperidinyl)ethyl]-7-(methyloxy)-1,5-naphthyridin-2(111)-one;
1-[2-(4-{[(5-chloro-6-methyl-3-pyridinyl)methyl]amino}-1-piperidinyl)ethyl]-7-(methyloxy)-1,5-naphthyridin-2(1H)-one;
1-[2-(4-{[(4-chloro-3-methylphenyl)methyl]amino}-1-piperidinyl)ethyl]-7-(methyloxy)-1,5-naphthyridin-2(1H)-one ;
1-[2-(4-{[(3-chloro-4-methylphenyl)methyl]amino}-1-piperidinyl)ethyl]-1,5-naphthyridin-2(1H)-one;
1-[2-((3R,4S)-4-{[(3,4-dichlorophenyl)methyl]amino}-3-hydroxy-1-piperidinyl)ethyl]-1,5-naphthyridin-2(1H)-one;
1-[2-(4-{[(3,4-dimethylphenyl)methyl]amino}-1-piperidinyl)ethyl]-7-fluoro-1,5-naphthyridin-2(1H)-one;
1-[2-(4-{[(4-chloro-3-methylphenyl)methyl]amino}-1-piperidinyl)ethyl]-1,5-naphthyridin-2(1H)-one;
1-[2-(4-{[(5-chloro-6-methyl-3-pyridinyl)methyl]amino}-1-piperidinyl)ethyl]-1,5-naphthyridin-2(1H)-one;
1-[2-(4-{[(5-bromo-6-methyl-2-pyridinyl)methyl]amino}-1-piperidinyl)ethyl]-7-fluoro-1,5-naphthyridin-2(1H)-one;
1-[2-(4-{[(3-fluoro-4-methylphenyl)methyl]amino}-1-piperidinyl)ethyl]-1,5-naphthyridin-2(1H)-one;
1-[2-(4-{[(3,4-dimethylphenyl)methyl]amino}-1-piperidinyl)ethyl]-1,5-naphthyridin-2(1H)-one;
1-[2-(4-{[(4-chloro-5-fluoro-2-pyridinyl)methyl]amino}-1-piperidinyl)ethyl]-7-fluoro-1,5-naphthyridin-2(1H)-one;
1-[2-(4-{[(5-bromo-2-furanyl)methyl]amino}-1-piperidinyl)ethyl]-7-fluoro-1,5-naphthyridin-2(1H)-one;
1-{2-[4-({[6-(trifluoromethyl)-3-pyridinyl]methyl}amino)-1-piperidinyl]ethyl}-1,5-naphthyridin-2(1H)-one;
1-{2-[4-({[4-chloro-3-(trifluoromethyl)phenyl]methyl}amino)-1-piperidinyl]ethyl}-1,5-naphthyridin-2(1H)-one;
7-(methyloxy)-1-[2-(4-{[(4-nitrophenyl)methyl]amino}-1-piperidinyl)ethyl]-1,5-naphthyridin-2(1H)-one;
1-[2-(4-{[(2,5-dichlorophenyl)methyl]amino}-1-piperidinyl)ethyl]-7-fluoro-1,5-naphthyridin-2(1H)-one;
1-[2-(4-{[(6-bromo-3-pyridinyl)methyl]amino}-1-piperidinyl)ethyl]-7-(methyloxy)-1,5-naphthyridin-2(1H)-one;
1-[2-(4-{[(3,5-dichlorophenyl)methyl]amino}-1-piperidinyl)ethyl]-7-fluoro-1,5-naphthyridin-2(1H)-one;
1-[2-(4-{[(4-fluorophenyl)methyl]amino}-1-piperidinyl)ethyl]-7-(methyloxy)-1,5-naphthyridin-2(1H)-one;
1-{2-[4-({[4-(trifluoromethyl)phenyl]methyl}amino)-1-piperidinyl]ethyl}-1,5-naphthyridin-2(1H)-one;
1-[2-(4-{[(4-chlorophenyl)methyl]amino}-1-piperidinyl)ethyl]-1,5-naphthyridin-2(1H)-one;
1-[2-(4-{[(5-chloro-2-thienyl)methyl]amino}-1-piperidinyl)ethyl]-7-fluoro-1,5-naphthyridin-2(1H)-one;
1-{2-[4-({[5-chloro-6-(methyloxy)-3-pyridinyl]methyl}amino)-1-piperidinyl]ethyl}-7-fluoro-1,5-naphthyridin-2(1H)-one;
1-[2-(4-[(3,5-dichlorophenyl)methyl]amino}-1-piperidinyl)ethyl]-7-(methyloxy)-1,5-naphthyridin-2(1H)-one ;
1-{2-[4-({[5-chloro-6-(methyloxy)-3-pyridinyl]methyl}amino)-1-piperidinyl]ethyl}-7-(methyloxy)-1,5-naphthyridin-2(1H)-one;
1-[2-(4-{[(5-bromo-2-thienyl)methyl]amino}-1-piperidinyl)ethyl]-1,5-naphthyridin-2-(1H)-one;
1-[2-(4-{[(2-bromo-1,3-thiazol-5-yl)methyl]amino}-1-piperidinyl)ethyl]-1,5-naphthyridin-2(1H)-one;
1-[2-(4-{[(4,5-dibromo-2-thienyl)methyl]amino}-1-piperidinyl)ethyl]-7-(methloxy)-1,5-naphthyridin-2(1H)-one;
1-{2-[4-({[3-chloro-4-(methyloxy)phenyl]methyl}amino)-1-piperidinyl]ethyl}-1,5-naphthyridin-2(1H)-one;
1-[2-(4-{[(3,4-dibromophenyl)methyl]amino}-1-piperidinyl)ethyl]-1,5-naphthyridin-2(1H)-one;
4-{[(1-{2-[7-(methyloxy)-2-oxo-1,5-naphthyridin-1(2H)-yl]ethyl}-4-piperidinyl)amino]methyl}benzonitrile;
1-[2-(4-{[(5-bromo-2-thienyl)methyl]amino}-1-piperidinyl)ethyl]-7-(methyloxy)-1,5-naphthyridin-2(1H)-one;
1-[2-(4-{[(4,5- dibromo-2-thienyl)methyl]amino}-1-piperidinyl)ethyl]-1,5-naphthyridin-2(1H)-one;
1-[2-(4-{[(5-bromo-4-methyl-2-thienyl)methyl]amino}-1-piperidinyl)ethyl]-7-fluoro-1,5-naphthyridin-2(1H)-one;
1-[2-(4-{[(3-chlorophenyl)methyl]amino}-1-piperidinyl)ethyl]-1,5-naphthyridin-2(1H)-one;
1-[2-(4-{[(3,5-dichlorophenyl)methyl]amino}-1-piperidinyl)ethyl]-1,5-naphthyridin-2(1H)-one;
7-fluoro-1-[2-(4-{[(4-methyl-3-nitrophenyl)methyl]amino}-1-piperidinyl)ethyl]-1,5-naphthyridin-2(1H)-one;
1-[2-(4-{[(4-bromo-1,3-thiazol-2-yl)methyl]amino}-1-piperidinyl)ethyl]-7-fluoro-1,5-naphthyridin-2(1H)-one;
1-[2-(4-{[(3-chloro-4-methylphenyl)methyl]amino}-1-piperidinyl)ethyl]-7-fluoro-1,5-naphthyridin-2(1H)-one;
7-fluoro-1-{2-[4-({[5-fluoro-6-(methyloxy)-3-pyridinyl]methyl}amino)-1piperidinyl]ethyl}-1,5-naphthyridin-2(1H)-one;
1-[2-(4-{[(5-bromo-2-furanyl)methyl]amino}-1-piperidinyl)ethyl]-7-(methyloxy)-1,5-naphthyridin-2(1H)-one;
1-[2-(4-{[(4-bromo-1,3-thiazol-2-yl)methyl]amino}-1-piperidinyl)ethyl]-7-(methyloxy)-1,5-naphthyridin-2(1H)-one;
1-[2-(4-{[(5-bromo-4-methyl-2-thienyl)methyl]amino}-1-piperidinyl)ethyl]-1,5-naphthyridin-2(1H)-one;
1-{2-[4-({[5-fluoro-6-(methyloxy)-3-pyridinyl]methyl}amino)-1-piperidinyl]ethyl}-7-(methyloxy)-1,5-naphthyridin-2(1H)-one;
7-(methyloxy)-1-{2-[4-({[6-(methyloxy)-3-pyridinyl]methyl}amino)-1-piperidinyl]ethyl}-1,5-naphthyridin-2(1H)-one;
1-[2-(4-{[(5-fluoro-6-methyl-2-pyridinyl)methyl]amino}-1-piperidinyl)ethyl]-7-(methyloxy)-1,5-naphthyridin-2(1H)-one;
1-[2-(4-{[(3-chloro-4-methylphenyl)methyl]amino}-1-piperidinyl)ethyl]-7-(methyloxy)-1,5-naphthyridin-2(1H)-one;
1-[2-(4-{[(6-fluoro-5-methyl-3-pyridinyl)methyl]amino}-1-piperidinyl)ethyl]-7-(methyloxy)-1,5-naphthyridin-2(1H)-one;
1-[2-(4-{[(4-chloro -5-fluoro-2-pyridinyl)methyl]amino}-1-piperidinyl)ethyl]-7-(methyloxy)-1,5-naphthyridin-2(1H)-one;
1-[2-(4-{[(5-bromo-4-methyl-1,3-thiazol-2-yl)methyl]amino}-1-piperidinyl)ethyl]-7-fluoro-1,5-naphthyridin-2(1H)-one;
1-[2-(4-{[(3,5-Dimethylphenyl)methyl]amino}-1-piperidinyl)ethyl]-7-(methyloxy)-1,5-naphthyridin-2(1H)-one;
1-[2-(4-{[(3,4-Dimethylphenyl)methyl]amino}-1-piperidinyl)ethyl]-7-(methyloxy)-1,5-naphthyridin-2(1H)-one;
1-[2-(4-{[(3-fluoro-5-methylphenyl)methyl]amino}-1-piperidinyl)ethyl]-7-(methyloxy)-1,5-naphthyridin-2(1H)-one;
1-[2-(4-{[(4-bromo-3-methylphenyl)methyl]amino}-1-piperidinyl)ethyl]-7-(methyloxy)-1,5-naphthyridin-2(1H)-one;
1-[2-(4-{[(5,6-dichloro-3-pyridinyl)methyl]amino}-1-piperidinyl)ethyl]-7-(methyloxy)-1,5-naphthyridin-2(1H)-one;
1-[2-(4-{[(3-fluoro-4-methylphenyl)methyl]amino}-1-piperidinyl)ethyl]-7-(methyloxy)-1,5-naphthyridin-2(1H)-one;
1-[2-(4-{[(4-chloro-3-methylphenyl)methyl]amino}-1-piperidinyl)ethyl]-7-fluoro-1,5-naphthyridin-2(1H)-one;
1-[2-(4-{[(5-bromo-3-pyridinyl)methyl]amino}-1-piperidinyl)ethyl]-7-fluoro-1,5-naphthyridin-2(1H)-one;
1-[2-(4-{[(4-bromo-2-thienyl)methyl]amino}-1-piperidinyl)ethyl]-1,5-naphthyridin-2(1H)-one;
1-[2-(4-{[(3,4-dibromophenyl)methyl]amino}-1-piperidinyl)ethyl]-7-fluoro-1,5-naphthyridin-2(1H)-one ;
1-[2-(4-{[(5-bromo-2-thienyl)methyl]amino}-1-piperidinyl)ethyl]-7-fluoro-1,5-naphthyridin-2(1H)-one;
7-fluoro-1-{2-[4-({[4-(trifluoromethyl)phenyl]methyl}amino)-1-piperidinyl}-1,5-naphthyridin-2 (1H)-one;
1-[2-(4-[(3,4-difluorophenyl)methyl]amino}-1-piperidinyl)ethyl]-7-fluoro-1,5-naphthyridin-2(1H)-one;
1-[2-(4-{[(5-bromo-6-methyl-2-pyridinyl)methyl]amino}-1-piperidinyl)ethyl]-7-(methyloxy)-1,5-naphthyridin-2(1H)-one;
1-[2-(4-[(3,4-dibromophenyl)methyl]amino}-1-piperidinyl)ethyl]-7-(methyloxy)-1,5-naphthyridin-2(1H)-one;
7-(methyloxy)-1-{2-[4-({[4-(trifluoromethyl)phenyl]methyl}amino)-1-piperidinyl]ethyl}-1,5-naphthyridin-2(1H)-one;
1-[2-(4-[(3,4-difluorophenyl)methyl]amino}-1-piperidinyl)ethyl]-7-(methyloxy)-1,5-naphthyridin-2(1H)-one;
1-[2-(4-{[(3-chlorophenyl)methyl]amino}-1-piperidinyl)ethyl]-7-(methyloxy)-1,5-naphthyridin-2(1H)-one;
1-{2-[4-({[4-chloro-3-(trifluoromethyl)phenyl]methyl}amino)-1-piperidinyl]ethyl}-7-(methyloxy)-1,5-naphthyridin-2(1H)-one;
1-[2-(4-{[(2,4-dichlorophenyl)methyl]amino}-1-piperidinyl)ethyl]-7-(methyloxy)- 1,5-naphthyridin-2(1H)-one;
1-[2-(4-{[(5-bromo-2-pyridinyl)methyl]amino}-1-piperidinyl)ethyl]-7-(methyloxy)-1,5-naphthyridin-2(1H)-one;
1-[2-(4-{[(2-chloro-1,3-thiazol-5-yl)methyl]amino}-1-piperidinyl)ethyl]-7-(methyloxy)-1,5-naphthyridin-2(1H)-one;
1-[2-(4-{[(5-bromo-4-methyl-2-thienyl)methyl]amino}-1-piperidinyl)ethyl]-7-(methyloxy)-1,5-naphthyridin-2(1H)-one
1-[2-((3R,4S)-4-{[(3,4-dichlorophenyl)methyl]amino}-3-hydroxy-1-piperidinyl)ethyl]-7-fluoro-1,5-naphthyridin-2(1H)-one;
1-{2-[(3R,4S)-3-hydroxy-4-({[6-(trifluoromethyl)-3-pyridinyl]methyl}amino)-1-piperidinyl]ethyl}-7-(methyloxy)-1,5-naphthyridin-2(1H)-one;
1-[2-((3R,4S)-4-{[(3,4-dichlorophenyl)methyl]amino}-3-hydroxy-1-piperidinyl)ethyl]-7-(methyloxy)-1,5-naphthyridin-2(1H)-one;
1-{2-[(3S,4R)-3-hydroxy-4-({[6-(trifluoromethyl)-3-pyridinyl]methyl}amino)-1-piperidinyl]ethyl}-7-(methyloxy)-1,5-naphthyridin-2(1H)-one;
1-[2-((3S,4R)-4-{[(3,4-dichlorophenyl)methyl]amino}-3-hydroxy-1-piperidinyl)ethyl]-7-(methyloxy)-1,5-naphthyridin-2(1H)-one;
1-{2-[(3R,4S)-3-hydroxy-4-({[6-(trifluoromethyl)-3-pyridinyl]methyl}amino)-1-piperidinyl]ethyl}-1,5-naphthyridin-2(1H)-one;
7-fluoro-1-{2-[(3R,4S)-3-hydroxy-4-({[6-(trifluoromethyl)-3-pyridinyl]methyl}amino)-1-piperidinyl]ethyl}-1,5-naphthyridin-2(1H)-one;
1-[2-(4-{[(3-chlorophenyl)methyl]amino}-1-piperidinyl)ethyl]-7-fluoro-1,5-naphthyridin-2(1H)-one;
1-[2-(4-{[(4-chlorophenyl)methyl]amino}-1-piperidinyl)ethyl]-7-fluoro-1,5-naphthyridin-2(1H)-one;
1-[2-(4-{[(4-bromo-2-thienyl)methyl]amino}-1-piperidinyl)ethyl]-7-(methyloxy)-1,5-naphthyridin-2(1H)-one;
1-[2-(4-{[(5-chloro-2-thienyl)methyl]amino}-1-piperidinyl)ethyl]-7-(methyloxy)-1,5-naphthyridin-2(1H)-one;
1-[2-(4-{[(2-bromo-1,3-thiazol-5-yl)methyl]amino}-1-piperidinyl)ethyl]-7-fluoro-1,5-naphthyridin-2(1H)-one;
1-[2-(4-[(4,5-dibromo-2-thienyl)methyl]amino}-1-piperidinyl)ethyl]-7-fluoro-1,5-naphthyridin-2(1H)-one;
1-[2-(4-{[(4-bromo-2-thienyl)methyl]amino}-1-piperidinyl)ethyl]-7-fluoro-1,5-naphthyridin-2(1H)-one;
7-(methyloxy)-1-{2-[4-({[5-(methyloxy)-6-nitro-2-pyridinyl]methyl}amino)-1- piperidinyl]ethyl}-1,5-naphthyridin-2(1H)-one;
1-[2-(4-{[(6-chloro-2-pyrazinyl)methyl]amino}-1-piperidinyl)ethyl]-7-(methyloxy)-1,5-naphthyridin-2(1H)-one;
2-fluoro-5-{[1-{2-[7-(methyloxy)-2-oxo-1,5-naphthyridin-1(2H)-yl]ethyl}-4-piperidinyl)amino]methyl}benzonitrile;
7-(methyloxy)-1-{2-[4-({[2-(trifluoromethyl)-4-pyrimidinyl]methyl}amino)-1-piperidinyl]ethyl}-1,5-naphthyridin-2(1H)-one:
1-[2-(4-{[(4,6-dimethyl-2-pyridinyl)methyl]amino}-1-piperidinyl)ethyl]-7-(methyloxy)-1,5-naphthyridin-2(1H)-one;
1-[2-(4-{[(5-chloro-3-pyridinyl)methyl]amino}-1-piperidinyl)ethyl]-7-(methyloxy)-1,5-naphthyridin-2(1H)-one;
1-[2-(4-{[(5-chloro-2-pyridinyl)methyl]amino}-1-piperidinyl)ethyl]-7-(methyloxy)-1,5-naphthyridin-2(1H)-one;
1-[2-((3R,4S)-4-{[(5-chloro-6-methyl-3-pyridinyl)methyl]amino}-3-hydroxy-1-piperidinyl)ethyl]-7-(methyloxy)-1,5-naphthyridin-2(1H)-one;
7-fluoro-1-[2-(4-{[(2-methyl-1H-imidazol-4-yl)methyl]amino}-1-piperidinyl)ethyl]-1,5-naphthyridin-2(1H)-one trifluoroacetate;
2-fluoro-5-[({1-[2-(7-fluoro-2-oxo -1,5-naphthyridin-1(2H)-yl)ethyl]-4-piperidinyl}amino)methyl]benzonitrile;
1-{2-[4-({[4-(1,1-dimethylethyl)phenyl]methyl}amino)-1-piperidinyl]ethyl}-7-fluoro-1,5-naphthyridin-2(1H)-one ;
7-fluoro-1-[2-(4-{[(6-fluoro-5-methyl-3-pyridinyl)methyl]amino}-1-piperidinyl)ethyl]-1,5-naphthyridin-2(1H)-one;
1-[2-((3R,4S)-4-{[(5-chloro-6-methyl-3-pyridinyl)methyl]amino}-3-hydroxy-1-piperidinyl)ethyl]-7-fluoro-1,5-naphthyridin-2(1H)-one hydrochloride;
1-[2-(4-{[(5,6-dimethyl-3-pyridinyl)methyl]amino}-1-piperidinyl)ethyl]-7-fluoro-1,5-naphthyridin-2(1H)-one;
1-[2-(4-{[(5-bromo-6-methyl-3-pyridinyl)methyl]amino}-1-piperidinyl)ethyl]-7-fluoro-1,5-naphthyridin-2(1H)-one;
1-[2-(4-{[(5-chloro-6-ethyl-3-pyridinyl)methyl]amino}-1-piperidinyl)ethyl]-7-fluoro-1,5-naphthyridin-2(1H)-one;
7-fluoro-1-[2-((3R,4S)-4-{[(5-fluoro-6-methyl-3-pyridinyl)methyl]amino}-3-hydroxy-1-piperidinyl)ethyl]-1,5-naphthyridin-2(1H)-one;
1-[2-(4-{[(5,6-dichloro-3-pyridinyl)methyl]amino}-1-piperidinyl)ethyl]-7-fluoro-1,5-naphthyridin-2(1H)-one;
1-[2-((3R,4S)-4-{[(5-bromo-6-methyl-3-pyridinyl)methyl]amino}-3-hydroxy-1-piperidinyl)ethyl]-7-fluoro-1,5-naphthyridin-2(1H)-one;
7-fluoro-1-{2-[4-methyl-4-({[6-(trifluoromethyl)-3-pyridinyl]methyl}amino)-1-piperidinyl]ethyl}-1,5-naphthyridin-2(1H)-one;
1-[2-(4-{[(3,4-dichlorophenyl)methyl]amino}-4-methyl-1-piperidinyl)ethyl]-7-(methyloxy)-1,5-naphthyridin-2(1H)-one;
and,
1-[2-(4-{[(3,4-dichlorophenyl)methyl]amino}-4-methyl-1-piperidinyl)ethyl]-7-fluoro-1,5-naphthyridin-2(111)-one, or a pharmaceutically acceptable salt, solvate or N-oxide thereof.

18. A pharmaceutical composition comprising a compound of claim 1, or a pharmaceutically acceptable salt, solvate or N-oxide thereof, and one or more pharmaceutically acceptable carriers, excipients or diluents.

19. A method of treatment of tuberculosis in mammals, particularly in man, which method comprises the administration to a mammal in need of such treatment an effective amount of a compound of claim 1, or a pharmaceutically acceptable salt, solvate or N-oxide thereof.

20-29. (canceled)
